SEQUENCE LISTING (128 sequences)

SEQ ID NO: 1-8 - skipped (8 entries)

SEQ ID NO: 9 - protein, 25 aa, Artificial Sequence
  Description of Artificial Sequence: Synthetic peptide
MKKLAFAITAASGAAAVLSHHDAEA

SEQ ID NO: 10 - protein, 30 aa, Artificial Sequence
  Description of Artificial Sequence: Synthetic polypeptide
WLDNRAFSKKFVPVVMATSVALFFLNLAFA

SEQ ID NO: 11 - protein, 29 aa, Artificial Sequence
  Description of Artificial Sequence: Synthetic peptide
MAKKFNYKLPSMVALTLFGTAFTAHQANA

SEQ ID NO: 12 - protein, 27 aa, Artificial Sequence
  Description of Artificial Sequence: Synthetic peptide
MKKRFLSICTMTIAALATTTMVNTSYA

SEQ ID NO: 13 - protein, 30 aa, Artificial Sequence
  Description of Artificial Sequence: Synthetic polypeptide
NLKKQSKLILIFICIFTFFIMIIQSQFLMG

SEQ ID NO: 14 - protein, 26 aa, Artificial Sequence
  Description of Artificial Sequence: Synthetic peptide
MKIFKLTSLTLAALTLAFPFSHVAQA

SEQ ID NO: 15 - protein, 27 aa, Artificial Sequence
  Description of Artificial Sequence: Synthetic peptide
MKKTVIASTLAVSLGIAGYGLSGHEAH

SEQ ID NO: 16 - protein, 27 aa, Artificial Sequence
  Description of Artificial Sequence: Synthetic peptide
MKKNKFLVYLLSTALITPTFATQTAFA

SEQ ID NO: 17 - protein, 35 aa, Artificial Sequence
  Description of Artificial Sequence: Synthetic polypeptide
MKTRQNKYSIRKFSVGASSILIAALLFMGGGSAQA

SEQ ID NO: 18 - protein, 37 aa, Artificial Sequence
  Description of Artificial Sequence: Synthetic polypeptide
MKNNNETRRFSIRKYTVGVVSIITGITIFVSGQHAQA

SEQ ID NO: 19 - protein, 30 aa, Artificial Sequence
  Description of Artificial Sequence: Synthetic polypeptide
MKKKLSYMITIMLAFTLSLALGLFFNSAHA

SEQ ID NO: 20 - protein, 45 aa, Artificial Sequence
  Description of Artificial Sequence: Synthetic polypeptide
MKKRRQGPINKRVDFLSNKVNKYSIRKFTVGTASILVGATLMFGA

SEQ ID NO: 21 - protein, 27 aa, Artificial Sequence
  Description of Artificial Sequence: Synthetic peptide
MKKRFLSICTMTIAALATTTMVNTSYA

SEQ ID NO: 22 - protein, 29 aa, Artificial Sequence
  Description of Artificial Sequence: Synthetic peptide
MAKKFNYKLPSMVALTLFGTAFTAHQANA

SEQ ID NO: 23 - protein, 50 aa, Artificial Sequence
  Description of Artificial Sequence: Synthetic polypeptide
MIKKNNLLTKKKPIANKSNKYAIRKFTVGTASIVIGAALLFGLGHNEAKA

SEQ ID NO: 24 - protein, 30 aa, Artificial Sequence
  Description of Artificial Sequence: Synthetic polypeptide
MKPFKLIFISALMILIMTNATPISHLNAQA

SEQ ID NO: 25 - protein, 35 aa, Artificial Sequence; identical to SEQ ID NO: 17
  Description of Artificial Sequence: Synthetic polypeptide

SEQ ID NO: 26 - protein, 23 aa, Artificial Sequence
  Description of Artificial Sequence: Synthetic peptide
MNKFKFFIVFLILSLVFLQNEYA

SEQ ID NO: 27 - protein, 51 aa, Artificial Sequence
  Description of Artificial Sequence: Synthetic polypeptide
MINKKNNLLTKKKPIANKSNKYAIRKFTVGTASIVIGATLLFGLGHNEAKA

SEQ ID NO: 28 - protein, 26 aa, Artificial Sequence
  Description of Artificial Sequence: Synthetic peptide
MKKIATATIATAGIATFAFAHHDAQA

SEQ ID NO: 29 - protein, 28 aa, Artificial Sequence
  Description of Artificial Sequence: Synthetic peptide
MKNFSKFALTSIAALTVASPLVNTEVDA

SEQ ID NO: 30 - protein, 37 aa, Artificial Sequence; identical to SEQ ID NO: 18
  Description of Artificial Sequence: Synthetic polypeptide

SEQ ID NO: 31 - protein, 19 aa, Artificial Sequence
  Description of Artificial Sequence: Synthetic peptide
MRYLKRITIYISLLILVSG

SEQ ID NO: 32 - protein, 20 aa, Artificial Sequence
  Description of Artificial Sequence: Synthetic peptide
MKLMNKIIVPVTASALLLGA

SEQ ID NO: 33 - protein, 40 aa, Artificial Sequence
  Description of Artificial Sequence: Synthetic polypeptide
MKKIDSWLTKHGLKNRLTLVVIVIFIIFLILLFMFVNLSD

SEQ ID NO: 34 - protein, 19 aa, Artificial Sequence
  Description of Artificial Sequence: Synthetic peptide
MKKKALLPLFLGIMIFLAG

SEQ ID NO: 35 - protein, 28 aa, Artificial Sequence
  Description of Artificial Sequence: Synthetic peptide
MKKTVIASTLAVSLGIAGYGLSGHEAHA

SEQ ID NO: 36 - protein, 19 aa, Artificial Sequence
  Description of Artificial Sequence: Synthetic peptide
MSKFKSLLLLFGTLILLSG

SEQ ID NO: 37 - protein, 27 aa, Artificial Sequence
  Description of Artificial Sequence: Synthetic peptide
MKKTLVASSLAIGLGVVAGNAGHDAHA

SEQ ID NO: 38 - protein, 29 aa, Artificial Sequence
  Description of Artificial Sequence: Synthetic peptide
MAKKFNYKLPSMVALTLFGTAFTAHQANA

SEQ ID NO: 39 - protein, 30 aa, Artificial Sequence; identical to SEQ ID NO: 19
  Description of Artificial Sequence: Synthetic polypeptide

SEQ ID NO: 40 - protein, 18 aa, Artificial Sequence
  Description of Artificial Sequence: Synthetic peptide
MHKRLFITLLGFIILLAG

SEQ ID NO: 41 - protein, 19 aa, Artificial Sequence
  Description of Artificial Sequence: Synthetic peptide
MRYLKRITIYISLLILVSG

SEQ ID NO: 42 - protein, 25 aa, Artificial Sequence
  Description of Artificial Sequence: Synthetic peptide
MQKKYITAIIGTTALSALASTHAQA

SEQ ID NO: 43 - protein, 22 aa, Artificial Sequence
  Description of Artificial Sequence: Synthetic peptide
MKHSSKIIVFVSFLILTIFIGG

SEQ ID NO: 44 - protein, 20 aa, Artificial Sequence
  Description of Artificial Sequence: Synthetic peptide
MKKWQLVGTTVLGASVLLGA

SEQ ID NO: 45 - protein, 52 aa, Artificial Sequence
  Description of Artificial Sequence: Synthetic polypeptide
MGKRRQGPINKKVDFLPNKLNKYSIRKFTVGTASILLGSTLIFGSSSHEAKA

SEQ ID NO: 46 - protein, 26 aa, Artificial Sequence
  Description of Artificial Sequence: Synthetic peptide
MKKIATATIATAGIATFAFAHHDAQA

SEQ ID NO: 47 - protein, 45 aa, Artificial Sequence; identical to SEQ ID NO: 20
  Description of Artificial Sequence: Synthetic polypeptide

SEQ ID NO: 48 - protein, 27 aa, Artificial Sequence
  Description of Artificial Sequence: Synthetic peptide
MKKRFLSICTMTIAALATTTMVNTSYA

SEQ ID NO: 49 - protein, 35 aa, Artificial Sequence; identical to SEQ ID NO: 17
  Description of Artificial Sequence: Synthetic polypeptide

SEQ ID NO: 50 - protein, 28 aa, Artificial Sequence
  Description of Artificial Sequence: Synthetic peptide
MKNFSKFALTSIAALTVASPLVNTEVDA

SEQ ID NO: 51 - protein, 17 aa, Artificial Sequence
  Description of Artificial Sequence: Synthetic peptide
MKKVLASATILSLMLVG

SEQ ID NO: 52 - protein, 22 aa, Artificial Sequence
  Description of Artificial Sequence: Synthetic peptide
MKYYGKCISYISILILTFFIGG

SEQ ID NO: 53 - protein, 22 aa, Artificial Sequence
  Description of Artificial Sequence: Synthetic peptide
MKHSSKIIVFVSFLILTIFIGG

SEQ ID NO: 54 - protein, 30 aa, Artificial Sequence; identical to SEQ ID NO: 24
  Description of Artificial Sequence: Synthetic polypeptide

SEQ ID NO: 55 - protein, 19 aa, Artificial Sequence
  Description of Artificial Sequence: Synthetic peptide
MSKFKSLLLLFGTLILLSG

SEQ ID NO: 56 - protein, 27 aa, Artificial Sequence
  Description of Artificial Sequence: Synthetic peptide
MKKTLVASSLAIGLGVVAGNAGHDAHA

SEQ ID NO: 57 - protein, 19 aa, Artificial Sequence
  Description of Artificial Sequence: Synthetic peptide
MHYLKKVTIYISLLILVSG

SEQ ID NO: 58 - protein, 25 aa, Artificial Sequence
  Description of Artificial Sequence: Synthetic peptide
MQKKYITAIIGTTALSALASTHAQA

SEQ ID NO: 59 - protein, 22 aa, Artificial Sequence
  Description of Artificial Sequence: Synthetic peptide
MKHSKKLLLCISFLLITFFIGG

SEQ ID NO: 60 - protein, 26 aa, Artificial Sequence
  Description of Artificial Sequence: Synthetic peptide
MKKIATATIATAGIATFAFAHHDAQA

SEQ ID NO: 61 - protein, 19 aa, Artificial Sequence
  Description of Artificial Sequence: Synthetic peptide
MRYLKKVTIYISLLILVSG

SEQ ID NO: 62 - protein, 27 aa, Artificial Sequence
  Description of Artificial Sequence: Synthetic peptide
MKKRFLSICTMTIAALATTTMVNTSYA

SEQ ID NO: 63 - protein, 20 aa, Artificial Sequence
  Description of Artificial Sequence: Synthetic peptide
MKKWQLVGTTVLGASVLLGA

SEQ ID NO: 64 - protein, 29 aa, Artificial Sequence
  Description of Artificial Sequence: Synthetic peptide
MAKKFNYKLPSMVALTLFGTAFTAHQANA

SEQ ID NO: 65 - protein, 30 aa, Artificial Sequence; identical to SEQ ID NO: 19
  Description of Artificial Sequence: Synthetic polypeptide

SEQ ID NO: 66 - protein, 18 aa, Artificial Sequence
  Description of Artificial Sequence: Synthetic peptide
MHKRLFITLLGFIILLAG

SEQ ID NO: 67 - protein, 22 aa, Artificial Sequence
  Description of Artificial Sequence: Synthetic peptide
MRYLKKVTIYISLLILTIFIGG

SEQ ID NO: 68 - protein, 17 aa, Artificial Sequence
  Description of Artificial Sequence: Synthetic peptide
MKKVLASATILSLMLVG

SEQ ID NO: 69 - protein, 22 aa, Artificial Sequence
  Description of Artificial Sequence: Synthetic peptide
MKHSKKLLLCISFLLITVFISG

SEQ ID NO: 70 - protein, 22 aa, Artificial Sequence
  Description of Artificial Sequence: Synthetic peptide
MKHSKKLLLCISFLLITFFISG

SEQ ID NO: 71 - protein, 28 aa, Artificial Sequence
  Description of Artificial Sequence: Synthetic peptide
MKKTVIASTLAVSLGIAGYGLSGHEAHA

SEQ ID NO: 72 - protein, 22 aa, Artificial Sequence
  Description of Artificial Sequence: Synthetic peptide
MKHSKKLLLCISFLLITIFISG

SEQ ID NO: 73 - protein, 40 aa, Artificial Sequence; identical to SEQ ID NO: 33
  Description of Artificial Sequence: Synthetic polypeptide

SEQ ID NO: 74 - protein, 19 aa, Artificial Sequence
  Description of Artificial Sequence: Synthetic peptide
MKKKALLPLFLGIMIFLAG

SEQ ID NO: 75 - protein, 20 aa, Artificial Sequence
  Description of Artificial Sequence: Synthetic peptide
MKLMNKIIVPVTASALLLGA

SEQ ID NO: 76 - protein, 35 aa, Artificial Sequence; identical to SEQ ID NO: 17
  Description of Artificial Sequence: Synthetic polypeptide

SEQ ID NO: 77 - protein, 13 aa, Artificial Sequence
  Description of Artificial Sequence: Synthetic peptide
GRKKRRQRRRPPQ

SEQ ID NO: 78 - protein, 27 aa, Artificial Sequence
  Description of Artificial Sequence: Synthetic peptide
GWTLNSAGYLLGKINLKALAALAKKIL

SEQ ID NO: 79 - protein, 18 aa, Artificial Sequence
  Description of Artificial Sequence: Synthetic peptide
KLALKLALKALKAALKLA

SEQ ID NO: 80 - protein, 30 aa, Artificial Sequence
  Description of Artificial Sequence: Synthetic polypeptide
WEAKLAKALAKALAKHLAKALAKALKACEA

SEQ ID NO: 81 - protein, 21 aa, Artificial Sequence
  Description of Artificial Sequence: Synthetic peptide
KETWWETWWTEWSQPKKKRKV

SEQ ID NO: 82 - protein, 9 aa, Artificial Sequence
  Description of Artificial Sequence: Synthetic peptide
RRRRRRRRR

SEQ ID NO: 83 - protein, 24 aa, Artificial Sequence
  Description of Artificial Sequence: Synthetic peptide
LGTYTQDFNKFHTFPQTAIGVGAP

SEQ ID NO: 84 - protein, 15 aa, Artificial Sequence
  Description of Artificial Sequence: Synthetic peptide
RQIKWFQNRRMKWKK

SEQ ID NO: 85 - protein, 11 aa, Artificial Sequence
  Description of Artificial Sequence: Synthetic peptide
YGRKKRRQRRR

SEQ ID NO: 86 - protein, 18 aa, Artificial Sequence
  Description of Artificial Sequence: Synthetic peptide
RGGRLSYSRRRFSTSTGR

SEQ ID NO: 87 - protein, 10 aa, Artificial Sequence
  Description of Artificial Sequence: Synthetic peptide
RRLSYSRRRF

SEQ ID NO: 88 - protein, 12 aa, Artificial Sequence
  Description of Artificial Sequence: Synthetic peptide
PIRRRKKLRRLK

SEQ ID NO: 89 - protein, 12 aa, Artificial Sequence
  Description of Artificial Sequence: Synthetic peptide
RRQRRTSKLMKR

SEQ ID NO: 90 - protein, 15 aa, Artificial Sequence
  Description of Artificial Sequence: Synthetic peptide
RRRRNRTRRNRRRVR

SEQ ID NO: 91 - protein, 19 aa, Artificial Sequence
  Description of Artificial Sequence: Synthetic peptide
KMTRAQRRAAARRNRWTAR

SEQ ID NO: 92 - protein, 13 aa, Artificial Sequence
  Description of Artificial Sequence: Synthetic peptide
TRRQRTRRARRNR

SEQ ID NO: 93 - protein, 13 aa, Artificial Sequence
  Description of Artificial Sequence: Synthetic peptide
GRKKRRQRRRPPQ

SEQ ID NO: 94 - protein, 13 aa, Artificial Sequence
  Description of Artificial Sequence: Synthetic peptide
GRRRRRRRRRPPQ

SEQ ID NO: 95 - protein, 27 aa, Artificial Sequence
  Description of Artificial Sequence: Synthetic peptide
GWTLNSAGYLLGKINLKALAALAKKIL

SEQ ID NO: 96 - protein, 17 aa, Artificial Sequence
  Description of Artificial Sequence: Synthetic peptide
KLALKLALKLALALKLA

SEQ ID NO: 97 - protein, 27 aa, Artificial Sequence
  Description of Artificial Sequence: Synthetic peptide
MGLGLHLLVLAAALQGAWSQPKKKRKV

SEQ ID NO: 98 - protein, 27 aa, Artificial Sequence
  Description of Artificial Sequence: Synthetic peptide
GALFLGWLGAAGSTMGAWSQPKKKRKV

SEQ ID NO: 99 - protein, 27 aa, Artificial Sequence
  Description of Artificial Sequence: Synthetic peptide
GALFLGFLGAAGSTMGAWSQPKKKRKV

SEQ ID NO: 100 - protein, 27 aa, Artificial Sequence
  Description of Artificial Sequence: Synthetic peptide
GALFLGFLGAAGSTMGAWSQPKSKRKV

SEQ ID NO: 101 - protein, 21 aa, Artificial Sequence
  Description of Artificial Sequence: Synthetic peptide
KETWWETWWTEWSQPKKKRKV

SEQ ID NO: 102 - protein, 21 aa, Artificial Sequence
  Description of Artificial Sequence: Synthetic peptide
KETWFETWFTEWSQPKKKRKV

SEQ ID NO: 103 - protein, 1064 aa, Homo sapiens
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 104 - protein, 55 aa, Homo sapiens
KNEDQEMCHEFQAFMKNGKLFCPQDKKFFQSLDGIMFINKCATCKMILEKEAKSQ

SEQ ID NO: 105 - protein, 63 aa, Homo sapiens
APTELNCDDFKKGERDGDFICPDYYEAVCGTDGKTYDNRCALCAENAKTGSQIGVKSEGECKS

SEQ ID NO: 106 - protein, 62 aa, Homo sapiens
NPEQDVCSAFRPFVRDGRLGCTRENDPVLGPDGKTHGNKCAMCAELFLKEAENAKREGETRI

SEQ ID NO: 107 - protein, 67 aa, Homo sapiens
NAEKDFCKEYEKQVRNGRLFCTRESDPVRGPDGRMHGNKCALCAEIFKQRFSEENSKTDQNLGKAEE

SEQ ID NO: 108 - protein, 62 aa, Homo sapiens
REIVKLCSQYQNQAKNGILFCTRENDPIRGPDGKMHGNLCSMCQAYFQAENEEKKKAEARAR

SEQ ID NO: 109 - protein, 68 aa, Homo sapiens
ESGKATSYAELCSEYRKLVRNGKLACTRENDPIQGPDGKVHGNTCSMCEVFFQAEEEEKKKKEGKSRN

SEQ ID NO: 110 - protein, 59 aa, Homo sapiens
ASFEELCSEYRKSRKNGRLFCTRENDPIQGPDGKMHGNTCSMCEAFFQQEERARAKAKR

SEQ ID NO: 111 - protein, 62 aa, Homo sapiens
EAAKEICSEFRDQVRNGTLICTREHNPVRGPDGKMHGNKCAMCASVFKLEEEEKKNDKEEKG

SEQ ID NO: 112 - protein, 62 aa, Homo sapiens
EAVQELCSEYRHYVRNGRLPCTRENDPIEGLDGKIHGNTCSMCEAFFQQEAKEKERAEPRAK

SEQ ID NO: 113 - protein, 63 aa, Homo sapiens
EAEKETCDEFRRLLQNGKLFCTRENDPVRGPDGKTHGNKCAMCKAVFQKENEERKRKEEEDQR

SEQ ID NO: 114 - protein, 57 aa, Homo sapiens
GNTQDECAEYREQMKNGRLSCTRESDPVRDADGKSYNNQCTMCKAKLEREAERKNEY

SEQ ID NO: 115 - protein, 63 aa, Homo sapiens
ESGKDTCDEFRSQMKNGKLICTRESDPVRGPDGKTHGNKCTMCKEKLEREAAEKKKKEDEDRS

SEQ ID NO: 116 - protein, 63 aa, Homo sapiens
NDKEDLCREFRSMQRNGKLICTRENNPVRGPYGKMHINKCAMCQSIFDREANERKKKDEEKSS

SEQ ID NO: 117 - protein, 62 aa, Homo sapiens
NNAKDECSEFRNYIRNNELICPRENDPVHGADGKFYTNKCYMCRAVFLTEALERAKLQEKPS

SEQ ID NO: 118 - protein, 62 aa, Homo sapiens
SLDSEMCKDYRVLPRIGYLCPKDLKPVCGDDGQTYNNPCMLCHENLIRQTNTHIRSTGKCEE

SEQ ID NO: 119 - DNA, 3192 bp, Homo sapiens
atgaagatagccacagtgtcagtgcttctgcccttggctctttgcctcatacaagatgctgccagtaagaatgaagatcaggaaatgtgccatgaatttcaggcatttatgaaaaatggaaaactgttctgtccccaggataagaaattttttcaaagtcttgatggaataatgttcatcaataaatgtgccacgtgcaaaatgatactggaaaaagaagcaaaatcacagaagagggccaggcatttagcaagagctcccaaggctactgccccaacagagctgaattgtgatgattttaaaaaaggagaaagagatggggattttatctgtcctgattattatgaagctgtttgtggcacagatgggaaaacatatgacaacagatgtgcactgtgtgctgagaatgcgaaaaccgggtcccaaattggtgtaaaaagtgaaggggaatgtaagagcagtaatccagagcaggatgtatgcagtgcttttcggccctttgttagagatggaagacttggatgcacaagggaaaatgatcctgttcttggtcctgatgggaagacgcatggcaataagtgtgcaatgtgtgctgagctgtttttaaaagaagctgaaaatgccaagcgagagggtgaaactagaattcgacgaaatgctgaaaaggatttttgcaaggaatatgaaaaacaagtgagaaatggaaggcttttttgtacacgggagagtgatccagtccgtggccctgacggcaggatgcatggcaacaaatgtgccctgtgtgctgaaattttcaagcagcgtttttcagaggaaaacagtaaaacagatcaaaatttgggaaaagctgaagaaaaaactaaagttaaaagagaaattgtgaaactctgcagtcaatatcaaaatcaggcaaagaatggaatacttttctgtaccagagaaaatgaccctattcgtggtccagatgggaaaatgcatggcaacttgtgttccatgtgtcaagcctacttccaagcagaaaatgaagaaaagaaaaaggctgaagcacgagctagaaacaaaagagaatctggaaaagcaacctcatatgcagagctttgcagtgaatatcgaaagcttgtgaggaacggaaaacttgcttgcaccagagagaacgatcctatccagggcccagatgggaaagtgcatggcaacacctgctccatgtgtgaggtcttcttccaagcagaagaagaagaaaagaaaaagaaggaaggtaaatcaagaaacaaaagacaatctaagagtacagcttcctttgaggagttgtgtagtgaataccgcaaatccaggaaaaacggacggcttttttgcaccagagagaatgaccccatccagggcccagatggaaaaatgcatggcaacacctgctccatgtgtgaggccttctttcaacaagaagaaagagcaagagcaaaggctaaaagagaagctgcaaaggaaatctgcagtgaatttcgggaccaagtgaggaatggaacacttatatgcaccagggagcataatcctgtccgtggcccagatggcaaaatgcatggaaacaagtgtgccatgtgtgccagtgtgttcaaacttgaagaagaagagaagaaaaatgataaagaagaaaaagggaaagtcgaggctgaaaaagttaagagagaagcagttcaggagctgtgcagtgaatatcgtcattatgtgaggaatggacgactcccctgtaccagagagaatgatcctattgagggtctagatgggaaaatccacggcaacacctgctccatgtgtgaagccttcttccagcaagaagcaaaagaaaaagaaagagctgaacccagagcaaaagtcaaaagagaagctgaaaaggagacatgcgatgaatttcggagacttttgcaaaatggaaaacttttctgcacaagagaaaatgatcctgtgcgtggcccagatggcaagacccatggcaacaagtgtgccatgtgtaaggcagtcttccagaaagaaaatgaggaaagaaagaggaaagaagaggaagatcagagaaatgctgcaggacatggttccagtggtggtggaggaggaaacactcaggacgaatgtgctgagtatcgggaacaaatgaaaaatggaagactcagctgtactcgggagagtgatcctgtacgtgatgctgatggcaaatcgtacaacaatcagtgtaccatgtgtaaagcaaaattggaaagagaagcagagagaaaaaatgagtattctcgctccagatcaaatgggactggatcagaatcagggaaggatacatgtgatgagtttagaagccaaatgaaaaatggaaaactcatctgcactcgagaaagtgaccctgtccggggtccagatggcaagacacatggcaataagtgtactatgtgtaaggaaaaactggaaagggaagcagctgaaaaaaaaaagaaagaggatgaagacaggagcaatacaggagaaaggagcaatacaggagaaaggagcaatgacaaagaggatctgtgtcgtgaatttcgaagcatgcagagaaatggaaagcttatctgcaccagagaaaataaccctgttcgaggcccatatggcaagatgcacatcaataaatgtgctatgtgtcagagcatctttgatcgagaagctaatgaaagaaaaaagaaagatgaagagaaatcaagtagcaagccctcaaataatgcaaaggatgagtgcagtgaatttcgaaactatataaggaacaatgaactcatctgccctagagagaatgacccagtgcacggtgctgatggaaagttctatacaaacaagtgctacatgtgcagagctgtctttctaacagaagctttggaaagggcaaagcttcaagaaaagccatcccatgttagagcttctcaagaggaagacagcccagactctttcagttctctggattctgagatgtgcaaagactaccgagtattgcccaggataggttatctttgtccaaaggatttaaagcctgtctgtggtgacgatggccaaacctacaacaatccttgcatgctctgtcatgaaaacctgatacgccaaacaaatacacacatccgcagtacagggaagtgtgaggagagcagcaccccaggaaccaccgcagccagcatgcccccgtctgacgaa

SEQ ID NO: 120 - protein, 6 aa, Artificial Sequence
  Description of Artificial Sequence: Synthetic 6xHis tag
HHHHHH

SEQ ID NO: 121 - protein, 4 aa, Artificial Sequence
  Description of Artificial Sequence: Synthetic peptide
AAPF

SEQ ID NO: 122 - DNA, 21 bp, Artificial Sequence
  Description of Artificial Sequence: Synthetic oligonucleotide
atgcgtcgtatgcgtcgtatg

SEQ ID NO: 123 - DNA, 18 bp, Artificial Sequence
  Description of Artificial Sequence: Synthetic oligonucleotide
gtggtggtggtggtggtg

SEQ ID NO: 124 - DNA, 22 bp, Artificial Sequence
  Description of Artificial Sequence: Synthetic oligonucleotide
ttgatatgcctcctaaattttt

SEQ ID NO: 125 - DNA, 21 bp, Artificial Sequence
  Description of Artificial Sequence: Synthetic oligonucleotide
taggcgcgcctattctaatgc

SEQ ID NO: 126 - protein, 17 aa, Artificial Sequence
  Description of Artificial Sequence: Synthetic peptide
  MISC_FEATURE (1)..(17): This sequence may encompass 4-17 residues
RRRRRRRRRRRRRRRRR

SEQ ID NO: 127 - protein, 17 aa, Artificial Sequence
  Description of Artificial Sequence: Synthetic peptide
  MISC_FEATURE (1)..(17): This sequence may encompass 4-17 residues
KKKKKKKKKKKKKKKKK

SEQ ID NO: 128 - DNA, 207 bp, Homo sapiens
gaatctggtaaagcaacgagttacgctgaattgtgctcagaataccgtaagttagtcagaaacggaaagttggcgtgtactcgtgagaacgaccctatccagggaccggacggcaaggtacatggaaatacttgcagtatgtgcgaggtgtttttccaggcggaggaagaagaaaagaagaaaaaggagggcaaaagtcgaaattaa

(19) United States
(12) Patent Application Publication
(10) Pub. No.: US 2021/0162029 A1
(43) Pub. Date: Jun. 3, 2021

(54) COMPOSITIONS AND METHODS FOR TREATMENT OR PREVENTION OF SKIN DISEASES AND DISORDERS WITH LEKTI

(71) Applicant: Azitra Inc, Branford, CT (US)

(72) Inventor: Mark N. Sampson, Doylestown, PA (US)

(21) Appl. No.: 17/089,220

(22) Filed: Nov. 4, 2020

Related U.S. Application Data
(60) Provisional application No. 62/930,307, filed on Nov. 4, 2019.
(60) Provisional application No. 62/930,313, filed on Nov. 4, 2019.
(60) Provisional application No. 62/930,312, filed on Nov. 4, 2019.

(51) Int. Cl. A61K 38/55; A61K 35/747; A61K 35/745; C12N 15/86

(52) U.S. Cl. CPC A61K 38/55; C12N 15/86; A61K 35/745; A61K 35/747

(57) ABSTRACT

The present disclosure provides, inter alia, treating and/or preventing skin diseases and disorders and symptoms thereof, using recombinant LEKTI domains and microbes genetically modified to express one or more LEKTI protein domains encoded by one or more SPINK genes. In certain embodiments, compositions, methods, and kits are provided comprising recombinant LEKTI domains and microbes genetically modified to express one or more LEKTI protein domains encoded by one or more SPINK genes.

### RELATED APPLICATIONS

This application claims benefit under 35 U.S.C. § 119(e) of U.S. Provisional Application No. 62/930,307 filed Nov. 4, 2019, U.S. Provisional Application No. 62/930,313 filed Nov. 4, 2019, and U.S. Provisional Application No. 62/930,312 filed Nov. 4, 2019, the contents of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 4, 2020, is named 129062-01302_SL.txt and is 59,748 bytes in size.

### FIELD OF THE DISCLOSURE

The present disclosure relates to methods, kits, and compositions for preventing or treating skin diseases or disorders in a subject, using one or more therapeutic LEKTI domains.

### BACKGROUND OF THE INVENTION

The epidermis, the squamous stratified epithelium of the skin, consists of multiple sublayers and is one of the most important barriers of the body against the outside world. The stratum corneum is the outermost layer of the epidermis and develops as a result of the final anucleated step in keratinocyte differentiation from the cells in nucleated epidermal layers. Although the stratum corneum is recognized as the most important physical barrier, the nucleated epidermal layers are also significant in barrier function (Proksch, Brandner et al. (2008) Exp Dermatol 17(12): 1063-1072). Together, the skin barrier protects against extensive water loss in one direction (inside-outside barrier) and against the invasion of harmful substances from the environment (outside-inside barrier) (Proksch, Brandner et al., 2008). The maintenance of the barrier is also important for balanced proliferation in the basal layer and preservation of the calcium ion gradient and thus proper epidermal differentiation (Lee, Jeong et al. (2006) Yonsei Med J 47(3): 293-306).

A number of current limitations exist in the treatment of skin. Many treatments, such as topical corticosteroids or biologics, do not treat the underlying issues of deficient intrinsic protein in the epidermis or imbalances in the microbial diversity in the skin. While recombinant proteins represent a promising group of therapeutic agents in the treatment of skin disease, several problems accompany their use in the context of the skin.

Traditional methods purify and concentrate recombinant proteins that are extracted from bacterial systems, and then incorporate such preparations into a delivery system. The purification of recombinant proteins is often a very costly method of obtaining protein. Moreover, a number of problems are associated with these traditional methods, including proteolytic degradation, inefficient delivery, and the need for repeated application overtime to achieve therapeutic effect.

Proteases or proteolytic enzymes are essential in organisms, from bacteria and viruses to mammals. Proteases digest and degrade proteins by hydrolyzing peptide bonds. Serine proteases (EC. 3.4.21) have common features in the active site, primarily an active serine residue. There are two main types of serine proteases; the chymotrypsin/trypsin/elastase-like or the subtilisin-like, which have an identical spatial arrangement of catalytic His, Asp, and Ser but in quite different protein scaffolds. Over twenty families (S1-S27) of serine proteases have been identified that are grouped into 6 clans on the basis of structural similarity and other functional evidence, SA, SB, SC, SE, SF & SG. The family of chymotrypsin/trypsin/elastase-like serine proteases have been subdivided into two classes. The “large” class (ca 230 residues) includes mostly mammalian enzymes such as trypsin, chymotrypsin, elastase, kallikrein, and thrombin. The “small” class (ca 190 residues) includes the bacterial enzymes. Examples of serine proteases include trypsin, tryptase, chymotrypsin, elastase, thrombin, plasmin, kallikrein, Complement Cl, acrosomal protease, lysosomal protease, cocoonase, a-lytic protease, protease A, protease B, serine carboxypeptidase t, subtilisin, urokinase (uPA), Factor Vila, Factor IXa, and Factor Xa. The serine proteases have been investigated widely, and are a major focus of research as a drug target due to their role in regulating a wide variety of physiological processes.

Serine protease inhibitors, or serpins, comprise a family of proteins that antagonize the activity of serine proteases. These proteins inhibit protease activity by a conserved mechanism involving a profound conformational change (as reviewed in Miranda and Lomas, 2006; Wang et al., 2008; and Ricagno et al., 2009). In this mechanism, the serpin presents a substrate-mimicking peptide sequence—the reactive center loop—to its target serine protease. Cleavage of the reactive center loop triggers a conformational change in which the bound protease translocates from the top to the bottom of the serpin molecule; simultaneously, part of the cleaved reactive center loop inserts into the β-sheet A of the serpin, thereby irreversibly inactivating the protease (Huntington et al., 2000; Briand et al., 2001).

One branch of the family of serine protease inhibitors is that of the Kazal type (SPINK) gene that includes SPINK1, SPINK2, SPINK4, SPINK5, SPINK6, SPINK7, SPINK8, SPINK9, SPINK13 and SPINK14). The lymphoepithelial kazal-type inhibitor (LEKTI) is a multi-domain serine protease inhibitor encoded by SPINK5 (Serine Proteinase Inhibitor Kazal type 5) (Magert et al. (1999) J Biol. Chem. 274; 21499-21502). The SPINK5 gene encoding LEKTI is located on chromosome 5 among a cluster of other SPINK genes (e.g., SPINK1, SPINK6, SPINK7, SPINK9 and SPINK13), and comprises 33 exons encoding 15 inhibitory domains separated by linker regions. SPINK5 has been shown to be expressed in the skin, oral mucosa, tonsils, parathyroid gland, thymus, and lung (Magert et al., Int J Biochem Cell Biol. 2002; 34(6):573-6; Magert et al., Eur J Med Res. 2002; 7(2):49-56).

SPINK5 stands out among the other SPINK genes for the large number of inhibitory domains it encodes. Additionally, the SPINK5 gene is transcribed into three different transcripts, resulting in three different LEKTI proteins that differ in the C-terminal region; i.e., a 145 kDa full length protein having inhibitory domains D1-D15, a 125 kDa (short) protein having inhibitory domains D1-D12, and a 148 kDa (long) protein having an extended linker region 13. LEKTI is expressed as high molecular mass precursors, which are rapidly processed into several proteolytic fragments secreted in the intercellular space (Bitoun et al. (2003) Hum. Mol. Genet. 12:2417-2430). The Kazal motif of LEKTI is defined by the presence of six cysteine residues positioned at specific distances to allow formation of three disulfide bonds in a 1-5, 2-4, and 3-6 pattern. Two of the domains of LEKTI (D2 and D5) form this six cysteine motif, while other domains share four cysteine residues, which produce a rigid inhibitory loop believed to mimic the substrate of target proteases and inactivate the target protease catalytic site. The LEKTI protein requires proteolytic cleavage for activation of its inhibitory function against many proteases. The full length protein is cleaved into domains D1-D5 and D6-D15. The D6-D15 domains are then further cleaved in multiple steps into D6-D9 and D10-D15,→D6 and D7-D9→D7 and D8-D9→D8. This process results in LEKTI proteins comprising between one and six inhibitory domains, with each protein having different inhibitory functions. For example, it has been shown that LEKTI fragments can efficiently and specifically inhibit the epidermal kallikrein (KLK) 5, KLK7, and KLK14 (DeRaison et al. (2007) Mol. Biol. Cell. 18:3607-3619).

It has been established that recombinant human LEKTI inhibits a battery of serine proteinases in vitro including plasmin, trypsin, cathepsin G, human KLKs, and elastase, enzymes implicated in the activation of MMPs (Jayakumar et al. (2014) MOJ Proteomics Bioinform; 1(5):124-128). A partial recombinant form of LEKTI containing domains 6-9 (rLEKTI6-9) has been shown to inhibit trypsin, subtilisin A, chymotrypsin, kallikrein 5 (KLK5), and kallikrein 7 (KLK7), but not plasmin, cathepsin G, or elastase (Jayakumar et al. (2004) Protein Expr. Purif 35, 93-101; Schechter et al. (2005) Biol. Chem. 386, 1173-1184). In addition, the single domain D6 was shown to be a potent inhibitor of trypsin, KLK5, and KLK7, whereas D15 was not effective against these two kallikreins (Egelrud et al. (2005) Br. J. Dermatol. 153, 1200-1203). Deraison et al. ((2007) Mol Biol Cell Vol. 18, 3607-3619) have identified KLK5 as a major target of LEKTI. Deraison et al. demonstrated that all LEKTI fragments, except D1, demonstrate specific and differential inhibition of human kallikreins 5, 7, and 14. Deraison et al. also found that the strongest inhibition was observed with D8-D11, toward KLK5, where kinetics analysis revealed an extremely tight binding complex. Thus, it has been reported that each form of LEKTI exhibits particular inhibitory specificity.

Kallikreins are a family of proteases consisting of 15 closely related, secreted serine proteases with either trypsin-like or chymotrypsin-like specificity, and are expressed in a variety of tissues such as prostate, ovary, breast, testis, brain, and skin. KLKs belong to a subgroup of the chymotrypsin-like serine protease family S1A of clan PA(S). The 15 human KLK genes are located on chromosome 19ql3.4 and constitute the largest contiguous serine protease cluster in the human genome. These genes, generally composed of five coding exons and in some cases one or two 5′ non-coding exons, encode the kallikrein-related peptidases KLK1 to KLK15. All KLK genes encode single-chain pre-pro-proteins containing a chymotrypsin- or trypsin-like catalytic domain of 224-237 residues with an amino acid sequence identity of approximately 40% among KLK4 to KLK15. KLK1, and its close homologs KLK2 and KLK3, form a clade of their own. KLK4, 5, and 7 belong to another subgroup, whereas KLK6 shares more similarity with KLK13 and KLK14. See Debela et al. (2008) Biol Chem 389, 623-632.

KLKs are colocalized with LEKTI in skin (Ekholm et al., J Invest Dermatol, 114 (2000), pp. 56-63; Bitoun et al. Hum Mol Genet, 12 (2003), pp. 2417-2430, 2003; Komatsu et al. Br J Dermatol, 153 (2005), pp. 274-281). In addition, KLKs and LEKTI are secreted together in lamellar bodies to the intercellular space, in the uppermost stratum granulosum (Sondell et al. J Invest Dermatol, 104 (1995), pp. 819-823; Ishida-Yamamoto et al. J Invest Dermatol, 122 (2004), pp. 1137-1144). KLKs are capable of cleaving corneodesmosomes, and their enzymatic activities are suppressed by partial recombinant LEKTI domains (Simon et al. J Biol Chem, 276 (2001), pp. 20292-20299; Caubet et al. J Invest Dermatol, 122 (2004), pp. 1235-1244; Egelrud et al. Br J Dermatol, 153 (2005), pp. 1200-1203; Schechter et al. Biol Chem, 386 (2005), pp. 1173-1184; Borgono et al. J Biol Chem, 282 (2007), pp. 3640-3652).

Imbalances in the proteolytic activity of KLKs, through gene over-expression or dysregulation of activity, is reported in a large number of skin disorders, including chronic itchy dermatitis, peeling skin syndrome, psoriasis, atopic dermatitis, and Netherton syndrome (Komatsu et al., 2005b; Descargues et al., 2005; Hachem et al., 2006; Komatsu et al., 2006; Hansson et al., 2002; Ekholm and Egelrud, 1999). The expression of multiple KLKs is significantly upregulated in psoriasis, atopic dermatitis, peeling skin syndrome type-B, and chronic lesions of atopic dermatitis (Komatsu et al., 2005b; Komatsu et al., 2006; Hansson et al., 2002). Patients with Netherton syndrome, an autosomal recessive skin disorder, have shown frame shifts and non-sense mutations in the SPINK5 gene encoding for LEKTI (Chavanas et al., 2000; Komatsu et al., 2002; Sprecher et al., 2001).

Serine proteases have a critical role in skin barrier function, and in the differentiation of keratinocytes. Serine protease activity has been shown to increase when skin surface pH is more alkali (Ekholm et al. (2000) J Invest Dermatol, 114, pp. 56-63; Hachem et al. (2005) J Invest Dermatol, 125, pp. 510-520; Mauro et al. (1998) Arch Dermatol Res, 290 (1998), pp. 215-222). KLK5 and KLK7 have been isolated and cloned from the stratum corneum (SC) (Hansson et al. (1994) J. Biol. Chem. 269, 19420-19426; Brattsand and Egelrud (1999) J. Biol. Chem. 274, 30033-40) and are the extracellular adhesion proteins of keratin desmosomes, desmoglein 1 and desmocollin 1. The serine protease kallikrein KLK5 serves as a dominant regulator of the protease cascade in the stratum corneum (SC) because it is capable of activating KLK7 (Caubet et al. (2004) J Invest Dermatol, 122, pp. 1235-1244) and KLK14 (Emami and Diamandis, (2008) J Biol Chem, 283, pp. 3031-3041), as well as self-activation (Ekholm and Egelrud (1998) Br J Dermatol, 139 (1998), pp. 585-590). In vitro studies have shown a potential activation mechanism of KLK7 by a proteolytic cascade involving KLK5 and KLK14 (Brattsand et al. (2005) J Invest Dermatol. 124, 198-203). KLKs are upregulated in inflammatory skin disorders including atopic dermatitis (AD) (Komatsu et al. (2007) Exp Dermatol, 16 (2007), pp. 513-519). The skin antibacterial action of KLK via regulating cathelicidin peptides has also been demonstrated in vitro and in vivo (Yamazaki et al. (2006) LFASEB J. 20, 2068-2080). In skin, KLK activities are regulated mainly by LEKTI in combination with changes in microenvironmental pH, as shown by in vitro studies (Deraison et al., 2007) and in Spink5−/− mice, an established animal model of Netherton Syndrome (NS).

Among the pathways activated by KLK5 are Par2, TSLP, Cathelicidin, TRPV ion channels and MMPs.

Proteolytic activities produced by zymogen activation via the KLK activome are involved in processes of skin desquamation, innate immunity, hypertension, semen liquefaction, neurodegeneration, and tumor-promoting or -inhibiting effects. Notably, certain KLKs exert pleiotropic functions by activating molecules involved in multiple processes, e.g. cathelicidin, which is involved in skin desquamation and innate immunity. Because KLK5 can activate itself as well as pro-KLK2, -3, -6, -7, -11, -12, and -14, KLK5 is considered the initiator of putative KLK cascades (Michael et al. (2006) J. Biol. Chem. 281, 12743-12750). Tissue kallikreins have been investigated in various diseases and disorders, including cancer, inflammation, pruritus and pain. A connection between the kallikrein-kinin system and inflammation has previously been established (Duchene, (2011) Kinins. De Gruyter. 261). Overexpression of various KLKs in the skin has led to the recognition that certain kallikrein inhibitors can be useful for certain dermatological conditions, including atopic dermatitis, psoriasis and rare skin diseases such as Netherton Syndrome (Freitas et al. Bioorganic & Medicinal Chemistry Letters 2012, 22, 6072-6075). A thorough discussion of tissue kallikreins, plasma kallikrein, their functions and potential roles in various diseases can be found in a variety of references, including the following which are incorporated herein by reference in their entireties and for all purposes: Renne, T.; Gruber, A. Thromb Haemost 2012, 107, 1012-3; Sotiropoulou, G.; Pampalakis, G. Trends in Pharmacological Sciences 2012, 33, 623-634; Pampalakis, G.; Sotiropoulou, G. Chapter 9 Pharmacological Targeting of Human Tissue Kallikrein-Related Peptidases. In Proteinases as Drug Targets, Dunn, B., Ed. The Royal Society of Chemistry: 2012; pp 199-228; Caliendo, G. et al., L. J Med Chem 2012, 55, 6669-86.

Also of interest is the potential involvement of kallikreins in the skin inflammation aspect of desquamation type disorders through activation of protease activated receptors (PARs). PARs 1-4 are G protein-coupled receptors, activated by various proteases including kallikreins. The PAR2 receptor has been shown to be involved in dermatitis, cell proliferation, cancer suppression, skin pigmentation, and skin moisture, and has been studied in the dermatology and cosmetic fields. PAR2 is activated by trypsin cleavage and coexists with tissue kallikrein in skin tissue. In skin lesions of subjects with atopic dermatitis and Netherton syndrome, the PAR2 receptor is overexpressed and has been shown to coexist with human tissue kallikrein (Descargues et al. (2006) J Invest Dermatol. 126 (7): 1622-32), implying that KLK causes inflammation of skin diseases through PAR2 activation. This lead to the hypothesis that such a KLK-PAR pathway is involved in the pathogenesis of these diseases and that KLKs induce inflammation in these skin disorders via PAR2 activation. It has been found that mutations in the SPINK5 gene result in upregulation of KLK5 activity which is involved in the formation of atopic dermatitis-like skin lesions via PAR-2 (Briot et al. 2009). Consistent with this, transgenic KLK5 overexpressor mice displayed signs of severe inflammation and pruritus (Furio et al. 2014).

Recent in vitro and in vivo work by Oikonomopoulou et al. (2006) has demonstrated that PAR activity may be targeted by active KLK5, 6, and 14. KLK5 and KLK6 were shown to activate PAR2, whereas KLK14 was reported to inactivate PAR1 and activate PAR2 and PAR4.

Thymic stromal lymphopoietin (TSLP) is a pleiotropic cytokine originally isolated from a murine thymic stromal cell line. TSLP exerts its biological effects by binding to a high-affinity heteromeric complex composed of thymic stromal lymphopoietin receptor chain and IL-7Rα. TSLP is primarily expressed by activated lung and intestinal epithelial cells, keratinocytes, and fibroblasts. Keratinocytes from lesional skin of atopic dermatitis patients have been shown to express TSLP. TSLP overexpression is also sufficient in mouse skin to induce an inflammatory Th2 microenvironment and an AD-like skin phenotype (Yoo et al. (2005) J. Exp. Med. 202:541-549). Briot et al. (2009, J. Exp. Med. Vol. 206 No. 5 1135-1147) have shown that KLK5 induces atopic dermatitis-like lesions through PAR2-mediated TSLP expression in Netherton syndrome.

Various proteases, such as KLKs, and also including mucunain, trypsin, or tryptase have been demonstrated to exert capacities as pruritogens in rodents or humans in vivo (Cormia and Dougherty J Invest Dermatol. 1960; 35:21-6; Hagermark Acta Derm Venereol. 1974; 54((5)):397-400; Stefansson et al. J Invest Dermatol. 2008; 128((1)):18-25; Steinhoff et al. J Neurosci. 2003; 23((15)):6176-80).

Cathelicidin proteins are composed of two distinct domains: an N-terminal “cathelin-like” or “prosequence” domain and the C-terminal domain of the mature anti-microbial peptide (AMP). The C-terminal domain of cathelicidins was among the earliest mammalian AMPs to show potent, rapid, and broad-spectrum killing activity. The term “cathelin-like” derives from the similarity of the N-terminal sequence with that of cathelin, a 12 kDa protein isolated from porcine neutrophils that shares similarity with the cystatin superfamily of cysteine protease inhibitors.

The C-terminal 37 amino acids of human cathelicidin (LL-37) has been characterized. LL-37 was originally referred to as FALL39, named for the first 4 N-terminal amino acids of this domain and the total number of residues (i.e., 39). LL-37 is a peptide predicted to contain an amphipathic alpha helix and lacks cysteine, making it different from all other previously isolated human peptide antibiotics of the defensin family, each of which contain 3 disulfide bridges. Full length human cathelicidin (sometimes referred to as full length LL-37) comprises the cathelin-like precursor protein and the C-terminal LL-37 peptide, thus comprising 170 amino acids. Mast cells (MCs) are one of the primary sources of Cath LL-37. Recent discoveries have indicated that an alteration in the metabolism of LL-37 Cathelicidin (Cath LL-37) antimicrobial peptide, by KLKs and MMPs activity, is present in the human inflammatory process that leads to rosacea.

Considerable side effects are associated with current treatments for skin diseases or disorders (e.g., steroid-containing ointments or antihistamines) commonly used to treat skin diseases or disorders. For example, long-term administration of topical or oral steroids thins the skin layer, causes osteoporosis, and inhibits the growth of children. In addition, when taking steroids is stopped, the lesions often recur.

Accordingly, there remains a need to develop improved therapeutics for the treatment, prevention or diagnosis of skin diseases and disorders, including inflammation, pain and pruritus.

### SUMMARY

The present disclosure is generally based on treating or preventing skin diseases and disorders.

According to some aspects, the disclosure is based on treating inflammatory diseases or disorders of the skin, by inhibiting pathways activated by protease targets of LEKTI.

According to some aspects, the disclosure is based on preventing inflammatory diseases or disorders of the skin, by inhibiting pathways activated by protease targets of LEKTI.

According to some aspects, the disclosure is based on treating pruritus by inhibiting pathways activated by protease targets of LEKTI.

According to some aspects, the disclosure is based on preventing pruritus by inhibiting pathways activated by protease targets of LEKTI. According to some embodiments, the pruritus is associated with the skin (the pruritus is a disease or disorder of the skin). According to some embodiments, the pruritus is not a disease or disorder of the skin (e.g., the pruritus is internal).

According to some aspects, the disclosure is based on treating pain by inhibiting pathways activated by protease targets of LEKTI.

According to some aspects, the disclosure is based on preventing pain by inhibiting pathways activated by protease targets of LEKTI.

The present disclosure is based, at least in part, on the finding that certain LEKTI fragments can selectively inhibit KLK5 activity, which in turn inhibits various proteases and proteolytic pathways implicated in inflammation, pruritus and/or pain.

According to one aspect, the disclosure provides a method of treating a skin disease or disorder in a subject in need thereof, comprising administering one or more LEKTI protein domains to the skin of the subject in need thereof, wherein the one or more LEKTI protein domains provides a therapeutic effect to decrease one or more symptoms of the skin disease or disorder.

According to another aspect, the disclosure provides a method of preventing a skin disease or disorder in a subject in need thereof, comprising administering one or more LEKTI protein domains to the skin of the subject in need thereof, wherein the one or more LEKTI protein domains provides a therapeutic effect to prevent one or more symptoms of the skin disease or disorder.

According to some embodiments, the skin disease or disorder is an inflammatory skin disease or disorder.

According to some embodiments, the skin disease or disorder is pruritus.

According to some embodiments, the skin disease or disorder is pain or manifests with a symptom of pain.

According to one aspect, the disclosure provides a method of treating an inflammatory skin disease or disorder in a subject in need thereof, comprising administering one or more LEKTI protein domains to the skin of the subject in need thereof, wherein the one or more LEKTI protein domains provides a therapeutic effect to decrease one or more symptoms of the inflammatory skin disease or disorder.

According to one aspect, the disclosure provides a method of preventing an inflammatory skin disease or disorder in a subject in need thereof, comprising administering one or more LEKTI protein domains to the skin of the subject in need thereof, wherein the one or more LEKTI protein domains provides a therapeutic effect to prevent one or more symptoms of the inflammatory skin disease or disorder.

According to one embodiment, the one or more LEKTI protein domains are encoded by a nucleic acid. According to some embodiments, the nucleic acid comprises SEQ ID NO: 119, or fragments thereof. According to some embodiments, the nucleic acid comprises a sequence that is at least 805, 85%, 90%, 95%, 96%, 97%, 98% or 99% identical to SEQ ID NO: 119. According to some embodiments, the nucleic acid comprises SEQ ID NO: 128, or fragments thereof. According to some embodiments, the nucleic acid comprises a sequence that is at least 805, 85%, 90%, 95%, 96%, 97%, 98% or 99% identical to SEQ ID NO: 128. According to some embodiments, the nucleic acid consists of SEQ ID NO: 119. According to some embodiments, the nucleic acid consists of SEQ ID NO: 128. According to another aspect, the nucleic acid is comprised in a vector. According to some embodiments, the vector is a viral expression vector. According to some embodiments, the vector is comprised within a cell.

According to one aspect, the disclosure provides a method of treating an inflammatory skin disease or disorder in a subject in need thereof, comprising administering a microbe genetically modified to express one or more LEKTI protein domains encoded by one or more SPINK genes to the skin of the subject in need thereof. According to some embodiments, the LEKTI protein domains penetrate the skin to provide a therapeutic effect to decrease one or more symptoms of the inflammatory skin disease or disorder.

According to one aspect, the disclosure provides a method of preventing an inflammatory skin disease or disorder in a subject in need thereof, comprising administering a microbe genetically modified to express one or more LEKTI protein domains encoded by one or more SPINK genes to the skin of the subject in need thereof. According to some embodiments, the LEKTI protein domains penetrate the skin to provide a therapeutic effect to prevent one or more symptoms of the inflammatory skin disease or disorder.

According to one embodiment of the above aspects, the one or more LEKTI protein domains are encoded by a nucleic acid. According to some embodiments, the nucleic acid comprises SEQ ID NO: 119, or fragments thereof. According to some embodiments, the nucleic acid comprises a sequence that is at least 805, 85%, 90%, 95%, 96%, 97%, 98% or 99% identical to SEQ ID NO: 119. According to some embodiments, the nucleic acid comprises SEQ ID NO: 128, or fragments thereof. According to some embodiments, the nucleic acid comprises a sequence that is at least 805, 85%, 90%, 95%, 96%, 97%, 98% or 99% identical to SEQ ID NO: 128. According to some embodiments, the nucleic acid consists of SEQ ID NO: 119. According to some embodiments, the nucleic acid consists of SEQ ID NO: 128. According to another aspect, the nucleic acid is comprised in a vector. According to some embodiments, the vector is a viral expression vector. According to some embodiments, the vector is comprised within a cell.

According to some embodiments of any one of the above aspects, the one or more symptoms are selected from one or more of inflammation, pain, itching, skin dryness, skin flaking, bacterial count, number of skin lesions, severity of skin lesions, frequency of outbreaks of skin lesions, redness, skin discoloration and expression of an inflammatory cytokine.

According to some embodiments of any one of the above aspects, the inflammatory skin disease or disorder is selected from the group consisting of rosacea, psoriasis and atopic dermatitis.

According to one aspect, the disclosure provides a method of preventing or reducing scarring associated with an inflammatory skin disease or disorder in a subject in need thereof, comprising administering one or more LEKTI protein domains to the skin of the subject in need thereof, wherein the one or more LEKTI protein domains penetrates the skin to provide a therapeutic effect to prevent or reduce scarring associated with the inflammatory skin disease or disorder.

According to one aspect, the disclosure provides a method of preventing or reducing scarring associated with an inflammatory skin disease or disorder in a subject in need thereof, comprising administering a microbe genetically modified to express one or more LEKTI protein domains encoded by one or more SPINK genes to the skin of the subject in need thereof. According to some embodiments, the LEKTI protein domains penetrates the skin to provide a therapeutic effect to prevent or reduce scarring associated with the inflammatory skin disease or disorder.

According to some embodiments, the inflammatory skin disease or disorder is selected from the group consisting of rosacea, psoriasis and atopic dermatitis (AD).

According to one aspect, the disclosure provides a method for decreasing the number of skin lesions in a subject with an inflammatory skin disease or disorder, comprising administering one or more LEKTI protein domains to the skin of the subject in need thereof, wherein the one or more LEKTI protein domains penetrates the skin to provide a therapeutic effect to decrease the number of skin lesions on skin of the subject.

According to one aspect, the disclosure provides a method for decreasing the number of skin lesions in a subject with an inflammatory skin disease or disorder, comprising administering a microbe genetically modified to express one or more LEKTI protein domains encoded by one or more SPINK genes to the skin of the subject in need thereof. According to some embodiments, the LEKTI protein domains penetrate the skin to provide a therapeutic effect to decrease the number of skin lesions on skin of the subject.

According to some embodiments of any one of the above aspects, the inflammatory skin disease or disorder is selected from the group consisting of rosacea, psoriasis and atopic dermatitis.

According to one aspect, the disclosure provides a method for inhibiting serine protease activity of at least one serine protease in a subject with an inflammatory skin disease or disorder, comprising administering one or more LEKTI protein domains to the skin of the subject in need thereof, wherein the one or more LEKTI protein domains penetrates the skin to provide a therapeutic effect to inhibit serine protease activity of at least one serine protease in the skin of the subject.

According to one aspect, the disclosure provides a method for inhibiting serine protease activity of at least one serine protease in a subject with an inflammatory skin disease or disorder, comprising administering a microbe genetically modified to express one or more LEKTI protein domains encoded by one or more SPINK genes to the skin of the subject in need thereof. According to some embodiments, the LEKTI protein domains penetrate the skin to provide a therapeutic effect to inhibit serine protease activity of at least one serine protease in the skin of the subject.

According to some embodiments of any one of the above aspects, the inflammatory skin disease or disorder is selected from the group consisting of rosacea, psoriasis and atopic dermatitis.

According to another aspect, the disclosure provides a method of treating or preventing pruritus in a subject, comprising administering one or more LEKTI protein domains to a subject to provide a therapeutic effect.

According to one aspect, the disclosure provides a method of treating or preventing pruritus in a subject, comprising administering a microbe genetically modified to express one or more LEKTI protein domains encoded by one or more SPINK gene to provide a therapeutic effect.

According to one aspect, the disclosure provides a method for treating or preventing itch as a symptom or sensation associated with a disease or disorder in a subject, comprising administering one or more LEKTI protein domains to provide a therapeutic effect.

According to one aspect, the disclosure provides a method for treating or preventing itch as a symptom or sensation associated with a disease or disorder in a subject, comprising administering one or more LEKTI protein domains to the skin of a subject in need thereof, wherein the one or more LEKTI protein domains penetrates the skin to provide a therapeutic effect.

According to one aspect, the disclosure provides a method of treating or preventing one or more symptom or sensation arising from an irritation, hives, pain, inflammation, asthma, allergy, or allergic rhinitis in a subject, comprising administering one or more LEKTI protein domains to provide a therapeutic effect.

According to one aspect, the disclosure provides a method of treating or preventing one or more symptom or sensation arising from an irritation, hives, pain, inflammation, asthma, allergy, or allergic rhinitis in a subject, comprising administering one or more LEKTI protein domains to the skin of a subject in need thereof, wherein the one or more LEKTI protein domains penetrates the skin to provide a therapeutic effect.

According to one aspect, the disclosure provides a method of treating or preventing pruritus in a subject, comprising administering a microbe genetically modified to express one or more LEKTI protein domains encoded by one or more SPINK gene to a subject to provide a therapeutic effect.

According to one aspect, the disclosure provides a method of treating or preventing pruritus in a subject, comprising administering a microbe genetically modified to express one or more LEKTI protein domains encoded by one or more SPINK gene, to provide a therapeutic effect.

According to one aspect, the disclosure provides a method for treating or preventing itch as a symptom or sensation associated with a disease or disorder in a subject, comprising administering a microbe genetically modified to express one or more LEKTI protein domains encoded by one or more SPINK gene to provide a therapeutic effect.

According to one aspect, the disclosure provides a method for treating or preventing itch as a symptom or sensation associated with a disease or disorder in a subject, comprising administering a microbe genetically modified to express one or more LEKTI protein domains encoded by one or more SPINK gene, to provide a therapeutic effect.

According to one aspect, the disclosure provides a method of treating or preventing one or more symptom or sensation arising from an irritation, hives, pain, inflammation, asthma, allergy, or allergic rhinitis in a subject, comprising administering a microbe genetically modified to express one or more LEKTI protein domains encoded by one or more SPINK gene to provide a therapeutic effect.

According to one aspect, the disclosure provides a method of treating or preventing one or more symptom or sensation arising from an irritation, hives, pain, inflammation, asthma, allergy, or allergic rhinitis in a subject, comprising administering a microbe genetically modified to express one or more LEKTI protein domains encoded by one or more SPINK gene to the skin of a subject in need thereof, to provide a therapeutic effect.

According to some embodiments of any of the above aspects, the therapeutic effect comprises decreasing itch or preventing itch. According to some embodiments of any of the above aspects, pruritus is caused by or associated with any condition or any treatment of a condition. According to some embodiments, the condition is a skin condition. According to some embodiments, the condition is a systemic condition.

According to one aspect, the disclosure provides a method for inhibiting serine protease activity of at least one serine protease in a subject with pruritus, comprising delivering one or more LEKTI protein domains to the skin of the subject in need thereof, wherein the one or more LEKTI protein domains penetrates the skin to provide a therapeutic effect to inhibit serine protease activity of at least one serine protease in the skin of the subject.

According to one aspect, the disclosure provides a method for inhibiting serine protease activity of at least one serine protease in a subject with pruritus, comprising delivering a microbe genetically modified to express one or more LEKTI protein domains encoded by one or more SPINK gene to the skin of the subject in need thereof, to provide a therapeutic effect to inhibit serine protease activity of at least one serine protease in the skin of the subject.

According to another further aspect, the disclosure provides a method of treating pain in a subject in need thereof, comprising administering one or more LEKTI protein domains to the subject in need thereof, to treat the pain.

According to one aspect, the disclosure provides a method of preventing pain in a subject in need thereof, comprising administering one or more LEKTI protein domains to a subject to prevent the pain.

According to one aspect, the disclosure provides a method of treating pain in a subject in need thereof, comprising administering one or more LEKTI protein domains to the skin of a subject in need thereof, wherein the one or more LEKTI protein domains penetrates the skin to treat the pain.

According to one aspect, the disclosure provides a method of preventing pain in a subject in need thereof, comprising administering one or more LEKTI protein domains to the skin of a subject in need thereof, wherein the one or more LEKTI protein domains penetrates the skin to prevent the pain.

According to one aspect, the disclosure provides a method for inhibiting serine protease activity of at least one serine protease in a subject suffering from pain, comprising administering one or more LEKTI protein domains to the skin of the subject in need thereof, wherein the one or more LEKTI protein domains penetrates the skin to provide a therapeutic effect to inhibit serine protease activity of at least one serine protease in the skin of the subject.

According to one aspect, the disclosure provides a method of treating pain in a subject in need thereof, comprising administering a microbe genetically modified to express one or more LEKTI protein domains encoded by one or more SPINK genes to the subject in need thereof, to treat the pain.

According to one aspect, the disclosure provides a method of preventing pain in a subject in need thereof, comprising administering a microbe genetically modified to express one or more LEKTI protein domains encoded by one or more SPINK genes to the subject in need thereof, to prevent the pain.

According to one aspect, the disclosure provides a method of treating pain in a subject in need thereof, comprising administering a microbe genetically modified to express one or more LEKTI protein domains encoded by one or more SPINK genes to the skin of the subject in need thereof. According to some embodiments, the microbe genetically modified to express one or more LEKTI protein domains encoded by one or more SPINK genes penetrates the skin to treat the pain. According to some embodiments, the one or more LEKTI domains expressed by the microbe penetrates the skin to treat the pain.

According to one aspect, the disclosure provides a method of preventing pain in a subject in need thereof, comprising administering a microbe genetically modified to express one or more LEKTI protein domains encoded by one or more SPINK genes the skin of the subject in need thereof. According to some embodiments, the microbe genetically modified to express one or more LEKTI protein domains encoded by one or more SPINK genes penetrates the skin to prevent the pain. According to some embodiments, the one or more LEKTI domains expressed by the microbe penetrates the skin to prevent the pain.

According to one aspect, the disclosure provides a method for inhibiting serine protease activity of at least one serine protease in a subject suffering from pain, comprising administering a microbe genetically modified to express one or more LEKTI protein domains encoded by one or more SPINK genes to the skin of the subject in need thereof. According to some embodiments, the microbe genetically modified to express one or more LEKTI protein domains encoded by one or more SPINK genes penetrates the skin to provide a therapeutic effect to inhibit serine protease activity of at least one serine protease in the skin of the subject. According to some embodiments, the one or more LEKTI domains expressed by the microbe penetrates the skin to inhibit serine protease activity of at least one serine protease in the skin of the subject.

According to some embodiments of any of the aspects herein, treating or preventing the pain comprises treating or preventing the symptoms of pain. According to some embodiments of any of the aspects herein, the pain is acute or chronic pain. According to some embodiments of any of the aspects herein, the pain is nociceptive pain. According to some embodiments of any of the aspects herein, the pain is neuropathic pain. According to some embodiments of any of the aspects herein, the pain is traumatic pain, inflammatory pain, post-operative incision pain, pain associated with cancer, neuropathic pain, fracture pain, osteoporotic fracture pain, bone cancer pain and gout joint pain.

According to some embodiments of any one of the above aspects, the serine protease is a kallikrein (KLK).

According to some embodiments, the kallikrein is KLK5.

According to some embodiments of any one of the above aspects, the method is part of a therapeutic regimen combining one or more additional treatment modalities.

According to some embodiments of any one of the above aspects, the microbe is adapted to live for a controlled duration on the surface of the mammal's skin to provide a continuous supply of LEKTI protein domains.

According to some embodiments of any one of the above aspects, the LEKTI protein domains are effective to ameliorate the symptoms of a skin disease or disorder.

According to some embodiments of any one of the above aspects, the microbe is genetically modified by transfection/transformation with a recombinant DNA plasmid encoding the LEKTI protein domains.

According to some embodiments of any one of the above aspects, the LEKTI domains are operably linked to one or more recombinant protein domains that are effective to enhance secretion from the microbe and/or penetration of the mammal's skin.

According to some embodiments of any one of the above aspects, at least one LEKTI domain is operably linked to a SecA domain.

According to some embodiments of any one of the above aspects, at least one LEKTI domain is operably linked to an RMR domain.

According to some embodiments of any of the above aspects, the one or more LEKTI protein domains are encoded by a nucleic acid. According to some embodiments, the nucleic acid comprises SEQ ID NO: 119, or fragments thereof. According to some embodiments, the nucleic acid comprises a sequence that is at least 80%, 85%, 90%, 95%, 96%, 97%, 98% or 99% identical to SEQ ID NO: 119. According to some embodiments, the nucleic acid comprises SEQ ID NO: 128, or fragments thereof. According to some embodiments, the nucleic acid comprises a sequence that is at least 80%, 85%, 90%, 95%, 96%, 97%, 98% or 99% identical to SEQ ID NO: 128. According to some embodiments, the nucleic acid consists of SEQ ID NO: 119. According to some embodiments, the nucleic acid consists of SEQ ID NO: 128. According to another aspect, the nucleic acid is comprised in a vector. According to some embodiments, the vector is a viral expression vector. According to some embodiments, the vector is comprised within a cell.

According to some embodiments of any one of the above aspects, the at least one LEKTI domain comprises any one of SEQ ID NOs 104-118 (any one of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118). According to some embodiments, the at least one LEKTI domain comprises an amino acid sequence that is at least 85% identical, at least 90% identical, at least 95% identical, at least 96% identical, at least 97% identical, at least 98% identical or at least 99% identical to SEQ ID NOs 104-118 (at least 85% identical, at least 90% identical, at least 95% identical, at least 96% identical, at least 97% identical, at least 98% identical or at least 99% identical to SEQ ID NO: 105, at least 85% identical, at least 90% identical, at least 95% identical, at least 96% identical, at least 97% identical, at least 98% identical or at least 99% identical to SEQ ID NO: 106, at least 85% identical, at least 90% identical, at least 95% identical, at least 96% identical, at least 97% identical, at least 98% identical or at least 99% identical to SEQ ID NO: 107, at least 85% identical, at least 90% identical, at least 95% identical, at least 96% identical, at least 97% identical, at least 98% identical or at least 99% identical to SEQ ID NO: 108, at least 85% identical, at least 90% identical, at least 95% identical, at least 96% identical, at least 97% identical, at least 98% identical or at least 99% identical to SEQ ID NO: 109, at least 85% identical, at least 90% identical, at least 95% identical, at least 96% identical, at least 97% identical, at least 98% identical or at least 99% identical to SEQ ID NO: 110, at least 85% identical, at least 90% identical, at least 95% identical, at least 96% identical, at least 97% identical, at least 98% identical or at least 99% identical to SEQ ID NO: 111, at least 85% identical, at least 90% identical, at least 95% identical, at least 96% identical, at least 97% identical, at least 98% identical or at least 99% identical to SEQ ID NO: 112, at least 85% identical, at least 90% identical, at least 95% identical, at least 96% identical, at least 97% identical, at least 98% identical or at least 99% identical to SEQ ID NO: 113, at least 85% identical, at least 90% identical, at least 95% identical, at least 96% identical, at least 97% identical, at least 98% identical or at least 99% identical to SEQ ID NO: 114, at least 85% identical, at least 90% identical, at least 95% identical, at least 96% identical, at least 97% identical, at least 98% identical or at least 99% identical to SEQ ID NO: 115, at least 85% identical, at least 90% identical, at least 95% identical, at least 96% identical, at least 97% identical, at least 98% identical or at least 99% identical to SEQ ID NO: 116, at least 85% identical, at least 90% identical, at least 95% identical, at least 96% identical, at least 97% identical, at least 98% identical or at least 99% identical to SEQ ID NO: 117 or at least 85% identical, at least 90% identical, at least 95% identical, at least 96% identical, at least 97% identical, at least 98% identical or at least 99% identical to SEQ ID NO: 118). According to some embodiments, the at least one LEKTI domain comprises SEQ ID NO: 109. According to some embodiments, the at least one LEKTI domain consists of SEQ ID NO: 109.

According to some embodiments of any one of the above aspects, at least one LEKTI domain comprises an amino acid sequence according to SEQ ID NO: 103.

According to some embodiments of any one of the above aspects, the microbe is adapted to multiply on the skin of the mammal.

According to some embodiments of any one of the above aspects, expression of at least one LEKTI domain is controlled by an operon and the amount of LEKTI provided to the subject's skin is proportional to the availability of an extrinsic factor.

According to some embodiments of any one of the above aspects, expression of at least one LEKTI domain is controlled by a promoter that is constitutively active.

According to some embodiments of any one of the above aspects, the microbe has been genetically modified by transfection/transformation with a recombinant DNA plasmid encoding the one or more LEKTI protein domains and one or more antibiotic resistance genes.

According to some embodiments of any one of the above aspects, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp. According to some embodiments, the microbe is Staphylococcus epidermidis.

According to one aspect, the disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide.

According to some embodiments, the disclosure provides a pharmaceutical composition comprising the recombinant microorganism.

### BRIEF DESCRIPTION OF THE DRAWINGS

FIG. 1 shows a vector construct comprising the therapeutic LEKTI domains of the present invention. The protein coding regions of the plasmid comprise SecA, 6×His tag (SEQ ID NO: 120), LEKTI D8-11, and RMR tag, operably linked to each other and under the control of a CmR promoter.

FIG. 2 shows a vector construct of the pJB38 plasmid according to some embodiments of the present invention.

FIG. 3 is a schematic showing the domains of the full length LEKTI polypeptide.

FIG. 4 is a panel of graphs that show LEKTI D6 inhibited KLK5 stimulation of human 3D skin construct, as determined by real time qPCR.

FIG. 5 is a graph that shows LEKTI D6 inhibited thymic stromal lymphopoietin (TSLP) expression, as determined by real time qPCR.

FIG. 6 is a graph that shows LEKTI D6 inhibited IL-6 expression in dendritic cells, as determined by real time qPCR.

FIG. 7A shows the results for TRPV1 agonist treatment and FIG. 7B shows the results for TRPV1 antagonist treatment in the activation assay described in Example 9.

FIG. 8A shows the results for TRPV4 agonist treatment and FIG. 8B shows the results for TRPV4 antagonist treatment in the activation assay described in Example 9.

### DETAILED DESCRIPTION OF THE INVENTION

According to one aspect, the present disclosure provides LEKTI proteins, or portions thereof, that are administered to a subject for the treatment of an inflammatory skin disease or disorder in a subject. According to another aspect, the disclosure provides LEKTI proteins, or portions thereof, that are administered to a subject for the treatment or prevention of pruritus in a subject. According to another aspect, the disclosure provides LEKTI proteins, or portions thereof, that are administered to a subject for the treatment or prevention of pain in a subject.

According to some embodiments of any of the above aspects, the disclosure provides skin-colonizing bacteria that are genetically modified to express one or more LEKTI protein domains encoded by one or more SPINK genes.

According to some embodiments, the LEKTI protein-producing bacteria are able to treat or prevent an inflammatory skin disease or disorder, by expressing and, optionally, secreting a therapeutic protein that treats the underlying cause of the disease or its symptoms.

According to some embodiments, the LEKTI protein-producing bacteria are able to treat or prevent pruritus, by expressing and, optionally, secreting a therapeutic protein that treats the underlying cause of the disease or its symptoms. According to some embodiments, the LEKTI protein-producing bacteria are able to treat or prevent itch as a symptom or sensation associated with a disease or disorder in a subject.

According to some embodiments, the therapeutic protein comprises one or more LEKTI domains that are effective to inhibit serine proteases within or on the skin of a mammal According to some embodiments, the recombinant LEKTI domains compensate for the defective endogenous LEKTI protein naturally produced by the skin in the mammal. According to some embodiments, the bacteria are able to self-replicate while retaining the ability to produce the recombinant protein, thereby providing a continuous supply of therapeutic agent.

According to some embodiments, the disclosure provides a composition for the treatment or prevention of an inflammatory skin disease or disorder comprising a microbe genetically modified to express one or more LEKTI protein domains encoded by one or more SPINK genes that is administered to the skin of a mammal, wherein the LEKTI protein domains are effective to penetrate one or more layers of the mammal's skin and effective to inhibit serine protease activity of at least one serine protease in or on the mammal's skin.

According to some embodiments, the disclosure provides a composition for the treatment or prevention of pruritus comprising a microbe genetically modified to express one or more LEKTI protein domains encoded by one or more SPINK genes that is administered to the skin of a mammal, wherein the LEKTI protein domains are effective to penetrate one or more layers of the mammal's skin and effective to inhibit serine protease activity of at least one serine protease in or on the mammal's skin.

### 1. Definitions

As used herein and in the appended claims, the singular forms “a,” “and,” and “the” include plural referents unless the context clearly dictates otherwise. Thus, for example, reference to “a polynucleotide” includes a plurality of such polynucleotides and reference to “the polypeptide” includes reference to one or more polypeptides, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The use of “or” means “and/or” unless stated otherwise Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “administration” as used herein is meant to refer to contact of a pharmaceutical composition, therapeutic composition, diagnostic agent or composition to a recipient, preferably a human.

As used herein, the terms “disease” or “disorder” are meant to refer to an impairment of health or a condition of abnormal functioning.

As used herein, an “effective amount” of an agent, e.g., a pharmaceutical formulation, is meant to refer to an amount effective, at dosages and for periods of time necessary, to achieve the desired therapeutic result.

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “functional variant of a gene” includes a variant of the gene with minor variations such as, for example, silent mutations, single nucleotide polymorphisms, missense mutations, and other mutations or deletions that do not significantly alter gene function.

As used herein, the term “gene delivery” means a process by which foreign DNA is transferred to host cells for applications of gene therapy.

As used herein, the term “gene of interest (GOI),” as used herein refers broadly to a heterologous sequence introduced into an expression vector, and typically refers to a nucleic acid sequence encoding a protein of therapeutic use in humans or animals.

The term “genetically modified” and grammatical variations thereof as used herein are meant to describe a microbial organism (e.g. bacteria) that has been genetically modified or engineered by the introduction of DNA prepared outside the microbe. For example, the introduction of plasmid DNA containing new genes into bacteria will allow the bacteria to express those genes. Alternatively, the DNA containing new genes can be introduced to the bacteria and then integrated into the bacteria's genome, where the bacteria will express those genes.

As used herein, the term “heterologous,” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide) Similarly, a cellular sequence (e.g., a gene or portion thereof) that is incorporated into a viral vector is a heterologous nucleotide sequence with respect to the vector.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, the term “infection,” is meant to refer broadly to delivery of heterologous DNA into a cell by a virus. The term “co-infection” as used herein means “simultaneous infection,” “double infection,” “multiple infection,” or “serial infection” with two or more viruses. Infection of a producer cell with two (or more) viruses will be referred to as “co-infection.” The term “transfection” refers to a process of delivering heterologous DNA to a cell by physical or chemical methods, such as plasmid DNA, which is transferred into the cell by means of electroporation, calcium phosphate precipitation, or other methods well known in the art.

The term “inhibiting” is meant to refer to the amount of a pharmaceutical composition as described herein that is sufficient to cause, for example, a decrease in KLK5 production or activity, protease production or activity, or a reduction in symptoms associated with an inflammatory skin disease or disorder (e.g., preventing or ameliorating a sign or symptoms of a disorder such as a rash, sore, and the like) as compared to a control subject or sample.

As used herein, the term “isolated” molecule (e.g., an isolated nucleic acid or protein or cell) means it has been identified and separated and/or recovered from a component of its natural environment.

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the LEKTI gene or portion thereof (e.g., LEKTI D6), or regulatory elements. A nucleic acid molecule can be single-stranded or double-stranded. A “LEKTI nucleic acid” refers to a nucleic acid that comprises the LEKTI gene or a portion thereof, or a functional variant of the LEKTI gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular LEKTI (e.g., LEKTI D6) protein is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

The term “operably linked” as used herein is meant to refer to the association of nucleic acid sequences on a single nucleic acid fragment so that the function of one is regulated by the other or is not hindered by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, two proteins can be operably linked, such that the function of either protein is not compromised. Generally, operably linked means that the nucleic acid sequences being linked are contiguous and, where necessary to join two protein coding regions, contiguous and in the same reading frame.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

The term “pharmaceutical formulation” as used herein is meant to refer to a preparation which is in such form as to permit the biological activity of an active ingredient contained therein to be effective, and which contains no additional components which are unacceptably toxic to a subject to which the formulation would be administered.

A “pharmaceutically acceptable carrier” as used herein is meant to refer to an ingredient in a pharmaceutical formulation, other than an active ingredient, which is nontoxic to a subject. A pharmaceutically acceptable carrier includes, but is not limited to, a buffer, excipient, stabilizer, or preservative.

The term “progression” as used herein refers to the course of a disease or disorder, such as a skin disease or disorder, as it becomes worse or spreads in the body.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. According to some embodiments, the promoter is selected from the group consisting of a CBA promoter, smCBA promoter, a CASI promoter, a GFAP promoter, and an elongation factor-1 alpha (EF1a) promoter. A “chicken beta-actin (CBA) promoter” refers to a polynucleotide sequence derived from a chicken beta-actin gene (e.g., Gallus beta actin, represented by GenBank Entrez Gene ID 396526). A “smCBA” promoter refers to the small version of the hybrid CMV-chicken beta-actin promoter. A “CASI” promoter refers to a promoter comprising a portion of the CMV enhancer, a portion of the chicken beta-actin promoter, and a portion of the UBC enhancer.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art.

The terms “pruritus” and “itch” as used herein are interchangeable and are meant to refer to an unpleasant cutaneous sensation which provokes the desire to scratch. Pruritus can occur in various levels of severity-mild pruritus, acute pruritus, and chronic pruritus, etc. Mild and acute pruritus, like pain, can serve a protective function, but chronic pruritus can have a significant negative impact on the quality of life of a subject. Pruritus may be widespread or localized on a subject's body.

The terms “protease” and “proteinase” as used herein are interchangeable, with both terms referring to an enzyme that performs proteolysis.

The term “quality of life” as used herein refers to the overall enjoyment of life, including aspects of an individual's sense of well-being and ability to carry out various activities.

The term “recombinant” and grammatical variations thereof are meant to relate to or denote an organism, protein, or genetic material formed by or using recombined DNA comprising DNA pieces from different sources or from different parts of the same source. For example, the term “recombinant DNA” means a DNA molecule formed through recombination methods to splice fragments of DNA from a different source or from different parts of the same source. According to some embodiments, two or more different sources of DNA are cleaved using restriction enzymes and joined together using ligases. As another example, the term “recombinant protein” or “recombinant domains” and grammatical variations thereof means a protein molecule formed through recombination methods originating from spliced fragments of DNA from a different source or from different parts of the same source. As another example, the term “recombinant microbe” or “recombinant bacteria” and grammatical variations thereof mean a microbe/bacteria that comprises one or more recombinant DNA/protein molecules.

The term “risk factor” as used herein refers to anything that raises the chances of a person developing a disease or disorder, such as a skin disease or disorder.

As used herein the term “secretory peptides” or “secretory sequences” or “secretion tags” or “signal peptides” or “export signals” and grammatical variations thereof means any peptide sequence that is capable of targeting the synthesized protein to the secretory pathway of a cell.

The term “skin” as used herein is meant to refer to the outer protective covering of the body of a mammal (e.g., a human), consisting of the corium and the epidermis, and is understood to include sweat and sebaceous glands, as well as hair follicle structures. Throughout the disclosure, the adjective “cutaneous” can be used, and should be understood to refer generally to attributes of the skin, as appropriate to the context in which they are used.

The term “skin disease or disorder” as used herein is meant to refer generally to symptoms affecting the skin. A skin disease or disorder may be a skin state or condition that is generally undesirable or deleterious compared to the normal or baseline condition of human skin. Examples of abnormal skin conditions include, without limitation, rosacea, psoriasis, atopic dermatitis, Netherton Syndrome, acne, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism. According to some embodiments, the skin disease or disorder is an inflammatory skin disease or disorder. According to some embodiments, the skin disease or disorder is rosacea, psoriasis or atopic dermatitis.

The term “subject” as used herein is meant to refer to a mammal Mammals include, but are not limited to, domesticated animals (e.g., cows, sheep, cats, dogs, and horses), primates (e.g., humans and non-human primates such as monkeys), rabbits, and rodents (e.g., mice and rats). According to some embodiments, the subject is a human. The term “subject” is used interchangeably with the term “patient” herein.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

As used herein, a “transgene expression cassette” or “expression cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The term “treatment” (and variations such as “treat” or “treating”) as used herein is meant to refer clinical intervention in an attempt to alter the natural course of the subject or cell being treated. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and improved prognosis.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “recombinant viral vector” is meant to refer to a recombinant polynucleotide vector comprising one or more heterologous sequences (i.e., nucleic acid sequence not of viral origin).

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

Transcriptional regulators refer to transcriptional activators and repressors that either activate or repress transcription of a gene of interest, such as LEKTI (e.g., LEKTI D6). Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### 2. Compositions

According to some aspects, the present disclosure provides compositions comprising a microbe genetically modified to express one or more LEKTI protein domains encoded by one or more SPINK genes. According to some embodiments, the one or more SPINK genes are selected from the group consisting of SPINK1, SPINK2, SPINK4, SPINK5, SPINK6, SPINK7, SPINK8, SPINK9, SPINK13, and SPINK14. According to some embodiments, the SPINK gene is SPINK5.

The SPINK gene can be obtained from any mammal, such as mouse, rat, rabbit, goat, sheep, horse, cow, dog, primate, or human gene sequences. According to some embodiments, the SPINK gene sequence is a human gene sequence.

According to some embodiments, the present disclosure also provides recombinant vectors containing a nucleotide sequence encoding one or more SPINK genes or portions thereof. Recombinant vectors include but are not limited to vectors useful for the expression of the open reading frames (ORFs) in E. coli, other bacteria, yeast, viral, baculovirus, plants or plant cells, as well as mammalian cells.

As described herein, according to some embodiments, the disclosure provides a microbe that is genetically modified to express one or more protein domains encoded by one or more SPINK genes to the subject in need thereof. According to some embodiments, the recombinant microbe is engineered to comprise a SPINK gene, or a fragment of the SPINK gene.

According to some embodiments, the nucleic acids have been appropriately modified, for example, by site directed mutagenesis, to remove sequences responsible for N-glycosylation not needed for biological activity. N-glycosylation sites in eukaryotic peptides are characterized by the amino acid sequence Asn-X-Ser/Thr where X is any amino acid except Pro. Modification of glycosylation sites can improve expression in for example yeast or mammalian cell cultures.

According to some embodiments, the nucleic acids have been modified to improve the production and solubility of recombinant protein in a suitable host which includes, but is not limited to removing cysteine residues unnecessary for intramolecular disulfide bond formation. cysteine residues may be changed by mutagenesis to another amino acid, for example serine, or removed from the sequence without affecting the biological activity or tertiary structure of the recombinant polypeptide.

Other modifications of the nucleic acids may be necessary to improve the stability and accumulation of the recombinant production of protein include but are not limited to mutations altering protease cleavage sites recognized by a suitable expression host. Such modifications can be made that will not adversely affect the biological activity or tertiary structure of the recombinant protein.

Additional modifications can be made to the nucleic acids that result in alterations in enzyme activity, substrate specificity, and/or biological activity. Such modifications may be preconceived based on specific knowledge relating to the protein or may be introduced by a random mutagenesis approach, for example error prone PCR. Additionally, it is also envisioned that one skilled in the art could generate chimeric nucleotide sequence comprising specific domains that can functionally replace stretches of nucleotide sequences that may add new function or improve the specificity or activity of the produced recombinant protein. According to some embodiments, modification resulting in changed biological activity of LEKTI may be necessary to improve the therapeutic effectiveness of the protein or to minimize potential side effects. Modification of the nucleic acid sequences can also be made that alter potential immunogenic sites that may result in allergic reactions to patients' administered with recombinant LEKTI protein.

Silent modifications can be made to the nucleic acids that do not alter, substitute or delete the respective amino acid in the recombinant protein. Such modification may be necessary to optimize, for example, the codon usage for a specific recombinant host. The nucleotide sequence of LEKTI or portions thereof can be modified to replace codons that are considered rare or have a low frequency of appropriate t-RNA molecules to a more suitable codon appropriate for the expression host. Such codon tables are known to exist and are readily available to one skilled in the art. In addition, silent modification can be made to the nucleic acid that minimizes secondary structure loops at the level of mRNA that may be deleterious to recombinant protein expression.

According to some embodiments, the one or more SPINK genes encodes a LEKTI protein, and protein domains thereof, selected from LEKTI, LEKTI-2 and LEKTI-3.

The present disclosure provides compositions comprising a therapeutically effective amount of a LEKTI polypeptide or a portion thereof.

According to some embodiments, a portion of a LEKTI polypeptide comprises one or more LEKTI protein domains. Accordingly, the present disclosure provides compositions comprising one or more LEKTI protein domains. Some non-limiting examples include one or more of domains D1, D2, D3, D4, D5, D6, D7, D8, D9, D10, D11, D12, D13, D14, and D15. According to some embodiments, the LEKTI domain comprises LEKTI inhibitory domain 6 (D6). According to some embodiments, the LEKTI domain comprises SEQ ID NO: 109.

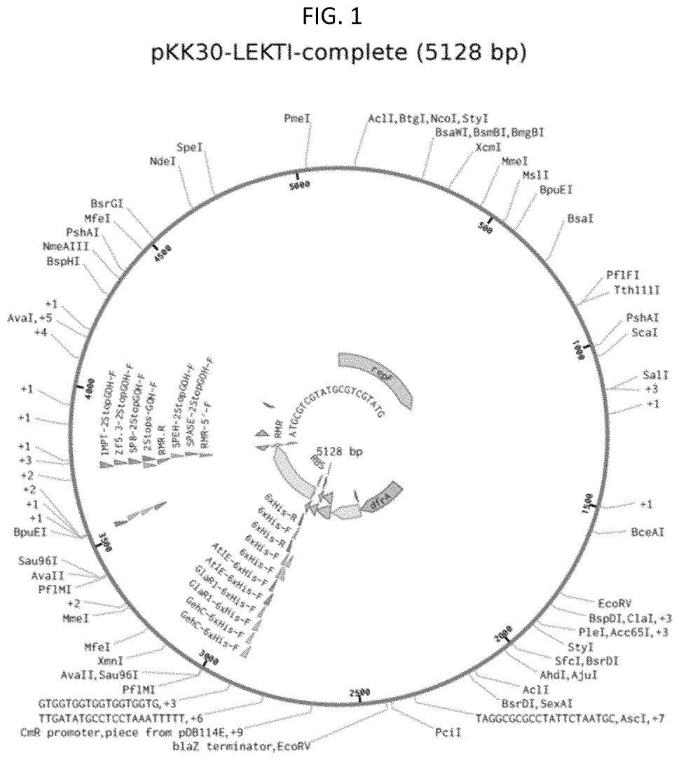

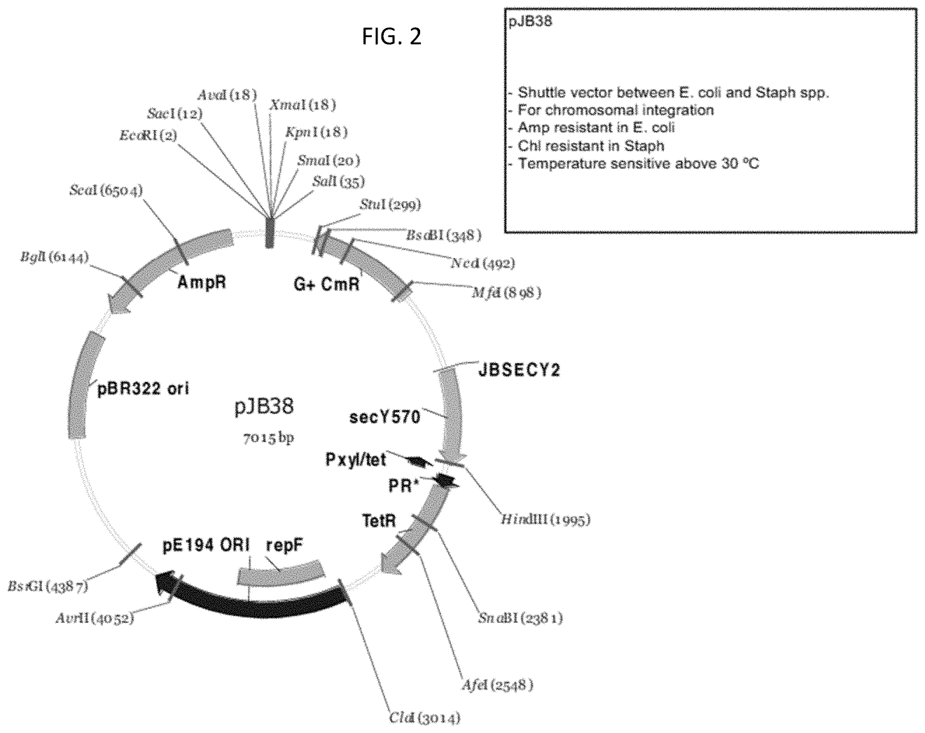

International Patent Application No. PCT/US2018/037850, incorporated by reference in its entirety herein, discloses various LEKTI recombinant proteins expressed by an engineered microbe. FIG. 1 from International Patent Application No. PCT/US2018/037850 shows a vector construct comprising the therapeutic LEKTI domains. The protein coding regions of the plasmid comprise SecA, 6×His tag (SEQ ID NO: 120), LEKTI D8-11, and RMR tag, operably linked to each other and under the control of a Cm^{R} promoter. FIG. 2 shows a vector construct of the pJB38 plasmid according to some embodiments of the present invention.

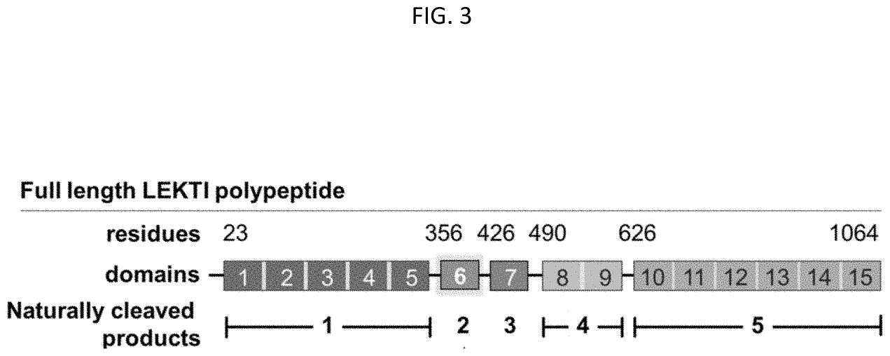

The LEKTI protein requires proteolytic cleavage for activation of its inhibitory function against many proteases. The full length protein is cleaved into domains D1-D5 and D6-D15. The D6-D15 domains are then further cleaved in multiple steps into D6-D9 and D10-D15,→D6 and D7-D9→D7 and D8-D9→D8. A schematic of the full-length LEKTI polypeptides, the domains and the naturally cleaved products is shown in FIG. 3 of International Patent Application No. PCT/US2018/037850, incorporated by reference in its entirety herein. The amino acid sequence of full length LEKTI protein is shown below as SEQ ID NO:103. Each of the 15 individual LEKTI domains are shown below as SEQ ID NOs 104-118:

LEKTI amino acid sequence Residues 1-1064 (SEQ ID NO: 103):

MKIATVSVLLPLALCLIQDAASKNEDQEMCHEFQAFMKNGKLFCPQDKKF
FQSLDGIMFINKCATCKMILEKEAKSQKRARHLARAPKATAPTELNCDDF
KKGERDGDFICPDYYEAVCGTDGKTYDNRCALCAENAKTGSQIGVKSEGE
CKSSNPEQDVCSAFRPFVRDGRLGCTRENDPVLGPDGKTHGNKCAMCAEL
FLKEAENAKREGETRIRRNAEKDFCKEYEKQVRNGRLFCTRESDPVRGPD
GRMHGNKCALCAEIFKQRFSEENSKTDQNLGKAEEKTKVKREIVKLCSQY
QNQAKNGILFCTRENDPIRGPDGKMHGNLCSMCQAYFQAENEEKKKAEAR
ARNKRESGKATSYAELCSEYRKLVRNGKLACTRENDPIQGPDGKVHGNTC
SMCEVFFQAEEEEKKKKEGKSRNKRQSKSTASFEELCSEYRKSRKNGRLF
CTRENDPIQGPDGKMHGNTCSMCEAFFQQEERARAKAKREAAKEICSEFR
DQVRNGTLICTREHNPVRGPDGKMHGNKCAMCASVFKLEEEEKKNDKEEK
GKVEAEKVKREAVQELCSEYRHYVRNGRLPCTRENDPIEGLDGKIHGNTC
SMCEAFFQQEAKEKERAEPRAKVKREAEKETCDEFRRLLQNGKLFCTREN
DPVRGPDGKTHGNKCAMCKAVFQKENEERKRKEEEDQRNAAGHGSSGGGG
GNTQDECAEYREQMKNGRLSCTRESDPVRDADGKSYNNQCTMCKAKLERE
AERKNEYSRSRSNGTGSESGKDTCDEFRSQMKNGKLICTRESDPVRGPDG
KTHGNKCTMCKEKLEREAAEKKKKEDEDRSNTGERSNTGERSNDKEDLCR
EFRSMQRNGKLICTRENNPVRGPYGKMHINKCAMCQSIFDREANERKKKD
EEKSSSKPSNNAKDECSEFRNYIRNNELICPRENDPVHGADGKFYTNKCY
MCRAVFLTEALERAKLQEKPSHVRASQEEDSPDSFSSLDSEMCKDYRVLP
RIGYLCPKDLKPVCGDDGQTYNNPCMLCHENLIRQTNTHIRSTGKCEESS
TPGTTAASMPPSDE

LEKTI Domains (and residues corresponding to the numbering of SEQ ID NO: 103) are set forth below:

LEKTI Domain 1
(residues 23-77; SEQ ID NO: 104)
KNEDQEMCHEFQAFMKNGKLFCPQDKKFFQSLDGIMFINKCATCKMILEK
EAKSQ
LEKTI Domain 2
(residues 91-153; SEQ ID NO: 105)
APTELNCDDFKKGERDGDFICPDYYEAVCGTDGKTYDNRCALCAENAKTG
SQIGVKSEGECKS
LEKTI Domain 3
(residues 155-216; SEQ ID NO: 106)
NPEQDVCSAFRPFVRDGRLGCTRENDPVLGPDGKTHGNKCAMCAELFLKE
AENAKREGETRI
LEKTI Domain 4
(residues 219-285; SEQ ID NO: 107)
NAEKDFCKEYEKQVRNGRLFCTRESDPVRGPDGRMHGNKCALCAEIFKQR
FSEENSKTDQNLGKAEE
LEKTI Domain 5
(residues 291-352; SEQ ID NO: 108)
REIVKLCSQYQNQAKNGILFCTRENDPIRGPDGKMHGNLCSMCQAYFQAE
NEEKKKAEARAR
LEKTI Domain 6
(residues 356-423; SEQ ID NO: 109)
ESGKATSYAELCSEYRKLVRNGKLACTRENDPIQGPDGKVHGNTCSMCEV
FFQAEEEEKKKKEGKSRN
LEKTI Domain 7
(residues 431-489; SEQ ID NO: 110)
ASFEELCSEYRKSRKNGRLFCTRENDPIQGPDGKMHGNTCSMCEAFFQQE
ERARAKAKR
LEKTI Domain 8
(residues 490-550; SEQ ID NO: 111)
EAAKEICSEFRDQVRNGTLICTREHNPVRGPDGKMHGNKCAMCASVFKLE
EEEKKNDKEEKG
LEKTI Domain 9
(residues 561622; SEQ ID NO: 112)
EAVQELCSEYRHYVRNGRLPCTRENDPIEGLDGKIHGNTCSMCEAFFQQE
AKEKERAEPRAK
LEKTI Domain 10
(residues 626-688; SEQ ID NO: 113)
EAEKETCDEFRRLLQNGKLFCTRENDPVRGPDGKTHGNKCAMCKAVFQKE
NEERKRKEEEDQR
LEKTI Domain 11
(residues 701-757; SEQ ID NO: 114)
GNTQDECAEYREQMKNGRLSCTRESDPVRDADGKSYNNQCTMCKAKLERE
AERKNEY
LEKTI Domain 12
(residues 768-830; SEQ ID NO: 115)
ESGKDTCDEFRSQMKNGKLICTRESDPVRGPDGKTHGNKCTMCKEKLERE
AAEKKKKEDEDRS
LEKTI Domain 13
(residues 843-905; SEQ ID NO: 116)
NDKEDLCREFRSMQRNGKLICTRENNPVRGPYGKMHINKCAMCQSIFDRE
ANERKKKDEEKSS
LEKTI Domain 14
(residues 910-970; SEQ ID NO: 117)
NNAKDECSEFRNYIRNNELICPRENDPVHGADGKFYTNKCYMCRAVFLTE
ALERAKLQEKPS
LEKTI Domain 15
(residues 987-1048; SEQ ID NO: 118)
SLDSEMCKDYRVLPRIGYLCPKDLKPVCGDDGQTYNNPCMLCHENLIRQT
NTHIRSTGKCEE
LEKTI nucleic acid sequence is set forth below as
SEQ ID NO: 119.
LEKTI Full length Nucleic acid sequence
(SEQ ID NO: 119)
ATGAAGATAGCCACAGTGTCAGTGCTTCTGCCCTTGGCTCTTTGCCTCAT
ACAAGATGCTCCAGTAAGAATGAAGATCAGGAAATGTGCCATGAATTTCA
GGCATTTATGAAAAATGGAAAACTGTTCTGTCCCCAGGATAAGAAATTTT
TTCAAAGTCTTGATGGAATAATGTTCATCAATAAATGTGCCACGTGCAAA
ATGATACTGGAAAAAGAAGCAAAATCACAGAAGAGGGCCAGGCATTTAGC
AAGAGCTCCCAAGGCTACTGCCCCAACAGAGCTGAATTGTGATGATTTTA
AAAAAGGAGAAAGAGATGGGGATTTTATCTGTCCTGATTATTATGAAGCT
GTTTGTGGCACAGATGGGAAAACATATGACAACAGATGTGCACTGTGTGC
TGAGAATGCGAAAACCGGGTCCCAAATTGGTGTAAAAAGTGAAGGGGAAT
GTAAGAGCAGTAATCCAGAGCAGGATGTATGCAGTGCTTTTCGGCCCTTT
GTTAGAGATGGAAGACTTGGATGCACAAGGGAAAATGATCCTGTTCTTGG
TCCTGATGGGAAGACGCATGGCAATAAGTGTGCAATGTGTGCTGAGCTGT
TTTTAAAAGAAGCTGAAAATGCCAAGCGAGAGGGTGAAACTAGAATTCGA
CGAAATGCTGAAAAGGATTTTTGCAAGGAATATGAAAAACAAGTGAGAAA
TGGAAGGCTTTTTTGTACACGGGAGAGTGATCCAGTCCGTGGCCCTGACG
GCAGGATGCATGGCAACAAATGTGCCCTGTGTGCTGAAATTTTCAAGCAG
CGTTTTTCAGAGGAAAACAGTAAAACAGATCAAAATTTGGGAAAAGCTGA
AGAAAAAACTAAAGTTAAAAGAGAAATTGTGAAACTCTGCAGTCAATATC
AAAATCAGGCAAAGAATGGAATACTTTTCTGTACCAGAGAAAATGACCCT
ATTCGTGGTCCAGATGGGAAAATGCATGGCAACTTGTGTTCCATGTGTCA
AGCCTACTTCCAAGCAGAAAATGAAGAAAAGAAAAAGGCTGAAGCACGAG
CTAGAAACAAAAGAGAATCTGGAAAAGCAACCTCATATGCAGAGCTTTGC
AGTGAATATCGAAAGCTTGTGAGGAACGGAAAACTTGCTTGCACCAGAGA
GAACGATCCTATCCAGGGCCCAGATGGGAAAGTGCATGGCAACACCTGCT
CCATGTGTGAGGTCTTCTTCCAAGCAGAAGAAGAAGAAAAGAAAAAGAAG
GAAGGTAAATCAAGAAACAAAAGACAATCTAAGAGTACAGCTTCCTTTGA
GGAGTTGTGTAGTGAATACCGCAAATCCAGGAAAAACGGACGGCTTTTTT
GCACCAGAGAGAATGACCCCATCCAGGGCCCAGATGGAAAAATGCATGGC
AACACCTGCTCCATGTGTGAGGCCTTCTTTCAACAAGAAGAAAGAGCAAG
AGCAAAGGCTAAAAGAGAAGCTGCAAAGGAAATCTGCAGTGAATTTCGGG
ACCAAGTGAGGAATGGAACACTTATATGCACCAGGGAGCATAATCCTGTC
CGTGGCCCAGATGGCAAAATGCATGGAAACAAGTGTGCCATGTGTGCCAG
TGTGTTCAAACTTGAAGAAGAAGAGAAGAAAAATGATAAAGAAGAAAAAG
GGAAAGTCGAGGCTGAAAAAGTTAAGAGAGAAGCAGTTCAGGAGCTGTGC
AGTGAATATCGTCATTATGTGAGGAATGGACGACTCCCCTGTACCAGAGA
GAATGATCCTATTGAGGGTCTAGATGGGAAAATCCACGGCAACACCTGCT
CCATGTGTGAAGCCTTCTTCCAGCAAGAAGCAAAAGAAAAAGAAAGAGCT
GAACCCAGAGCAAAAGTCAAAAGAGAAGCTGAAAAGGAGACATGCGATGA
ATTTCGGAGACTTTTGCAAAATGGAAAACTTTTCTGCACAAGAGAAAATG
ATCCTGTGCGTGGCCCAGATGGCAAGACCCATGGCAACAAGTGTGCCATG
TGTAAGGCAGTCTTCCAGAAAGAAAATGAGGAAAGAAAGAGGAAAGAAGA
GGAAGATCAGAGAAATGCTGCAGGACATGGTTCCAGTGGTGGTGGAGGAG
GAAACACTCAGGACGAATGTGCTGAGTATCGGGAACAAATGAAAAATGGA
AGACTCAGCTGTACTCGGGAGAGTGATCCTGTACGTGATGCTGATGGCAA
ATCGTACAACAATCAGTGTACCATGTGTAAAGCAAAATTGGAAAGAGAAG
CAGAGAGAAAAAATGAGTATTCTCGCTCCAGATCAAATGGGACTGGATCA
GAATCAGGGAAGGATACATGTGATGAGTTTAGAAGCCAAATGAAAAATGG
AAAACTCATCTGCACTCGAGAAAGTGACCCTGTCCGGGGTCCAGATGGCA
AGACACATGGCAATAAGTGTACTATGTGTAAGGAAAAACTGGAAAGGGAA
GCAGCTGAAAAAAAAAAGAAAGAGGATGAAGACAGGAGCAATACAGGAGA
AAGGAGCAATACAGGAGAAAGGAGCAATGACAAAGAGGATCTGTGTCGTG
AATTTCGAAGCATGCAGAGAAATGGAAAGCTTATCTGCACCAGAGAAAAT
AACCCTGTTCGAGGCCCATATGGCAAGATGCACATCAATAAATGTGCTAT
GTGTCAGAGCATCTTTGATCGAGAAGCTAATGAAAGAAAAAAGAAAGATG
AAGAGAAATCAAGTAGCAAGCCCTCAAATAATGCAAAGGATGAGTGCAGT
GAATTTCGAAACTATATAAGGAACAATGAACTCATCTGCCCTAGAGAGAA
TGACCCAGTGCACGGTGCTGATGGAAAGTTCTATACAAACAAGTGCTACA
TGTGCAGAGCTGTCTTTCTAACAGAAGCTTTGGAAAGGGCAAAGCTTCAA
GAAAAGCCATCCCATGTTAGAGCTTCTCAAGAGGAAGACAGCCCAGACTC
TTTCAGTTCTCTGGATTCTGAGATGTGCAAAGACTACCGAGTATTGCCCA
GGATAGGTTATCTTTGTCCAAAGGATTTAAAGCCTGTCTGTGGTGACGAT
GGCCAAACCTACAACAATCCTTGCATGCTCTGTCATGAAAACCTGATACG
CCAAACAAATACACACATCCGCAGTACAGGGAAGTGTGAGGAGAGCAGCA
CCCCAGGAACCACCGCAGCCAGCATGCCCCCGTCTGACGAA
LEKTI domain 6 (D6) nucleic acid sequence
(SEQ ID NO: 128)
gaatctggtaaagcaacgagttacgctgaattgtgctcagaataccgtaa
gttagtcagaaacggaaagttggcgtgtactcgtgagaacgaccctatcc
agggaccggacggcaaggtacatggaaatacttgcagtatgtgcgaggtg
ataccaggcggaggaagaagaaaagaagaaaaaggagggcaaaagtcgaa
atTAA

According to some embodiments, the disclosure relates to the full length LEKTI molecule, one or more of domains D1, D2, D3, D4, D5, D6, D7, D8, D9, D10, D11, D12, D13, D14, and D15, as well as isolated fragments, oligonucleotides, and truncations maintaining biological activity, for example N-terminal deletions, C-terminal deletions, or deletions at both N and C-termini derived from SEQ ID NO: 119 and deduced amino acid sequence SEQ ID NO: 103. The amino acid sequence of full length LEKTI protein is set forth as SEQ ID NO: 103, as well as each of the 15 individual domains as shown below.

The present disclosure also relates to allelic variants of LEKTI, or portions thereof (one or more of domains D1, D2, D3, D4, D5, D6, D7, D8, D9, D10, D11, D12, D13, D14, and D15), as well as synthetic or mutated genes of SPINK (e.g., SPINK5) that have been modified to change, for example, the expression or activity of the recombinant protein. It is also noted that degeneracy of the nucleic acid code can be considered variations in the nucleotide sequences that encode the same amino acid residues. Accordingly, the disclosure includes nucleic acid residues that are able to hybridize under moderately stringent conditions. One skilled in the art can determine effective combinations of salt and temperature to constitute a moderately stringent hybridization condition. It is also envisioned that orthologs of SPINK genes are present in other species, for example, dog, sheep, rat, hamster, chicken and pig. Therefore in another embodiment of the present invention relates to SPINK (e.g., SPINK5) nucleic acids that encode polypeptides having at least about 70% to 80% identity, preferably 90% to 95% identity (e.g., 90%, 91%, 92%, 93%, 94%, 95%), more preferably 98% to 99% identity to LEKTI set forth in SEQ ID NO: 103 or portions thereof (one or more of domains D1, D2, D3, D4, D5, D6, D7, D8, D9, D10, D11, D12, D13, D14, and D15).

The present disclosure also provides recombinant vectors containing a nucleotide sequence encoding SEQ ID NO: 103 or portions thereof (one or more of domains D1, D2, D3, D4, D5, D6, D7, D8, D9, D10, D11, D12, D13, D14, and D15). Recombinant vectors include but are not limited to vectors useful for the expression of the open reading frames (ORFs) in E. coli, yeast, viral, baculovirus, plants or plant cells, as well as mammalian cells.

Expression Systems Useful for Production of LEKTI or Portions Thereof

The present disclosure also provides for recombinant cloning and expression vectors useful for the production of biologically active LEKTI. Such expression plasmids may be used to prepare recombinant LEKTI polypeptides or portions thereof (one or more of domains D1, D2, D3, D4, D5, D6, D7, D8, D9, D10, D11, D12, D13, D14, and D15) encoded by the nucleic acids in a suitable host organism. Suitable host organisms for the production of LEKTI or portions thereof (one or more of domains D1, D2, D3, D4, D5, D6, D7, D8, D9, D10, D11, D12, D13, D14, and D15) include, but are not limited to bacteria, yeast, insect cells, mammalian cells, plants and plant cells. In addition, cell free systems may also be employed for the production of recombinant proteins. One skilled in the art can readily prepare plasmids suitable for the expression of recombinant LEKTI in the suitable host organism. Appropriate cloning and expression vectors are readily available to one skilled in the art and can be obtained from commercial sources or from the ATCC.

The recombinant protein can be produced in the within the host cell or secreted into the culture medium depending on the nature of the vector system used for the production of the recombinant protein. Generally plasmids useful for the expression of the recombinant LEKTI or portions thereof (one or more of domains D1, D2, D3, D4, D5, D6, D7, D8, D9, D10, D11, D12, D13, D14, and D15) comprise necessary operable linked regulatory elements such as a promoter sequence (including operators, enhancers, silencers, ribosomal binding sites), transcriptional enhancing sequences, translational fusions to signal peptides (native or heterologous) or peptide sequences useful for the purification of recombinant protein (for example His Tag, FLAG® (a convenient binding moiety), MBP, GST), transcription termination signals and poly adenylation signals (if necessary).

It may also be necessary for the recombinant plasmid to replicate in the host cell. This requires the use of an origin of replication suitable for the host organism. Alternatively, the recombinant expression plasmid may be stably integrated into the host's chromosome. This may require homologous recombination or random integration into the host chromosomes. Both instances require the use of an appropriate selection mechanism to distinguish transformed host cells from non-transformed host cells. Useful selection schemes include the use of, for example, antibiotics (for example, G418, ZEOCIN® (a glycopeptide antibiotic of the bleomycin family), kanamycin, tetracycline, gentamycin, spectinomycin, ampicillin), complementation of an auxotroph (for example Trp-, DHFR-), and scorable markers (for example β-glucoronidase, β-galactosidase, GFP).

Expression systems useful in the present invention include yeast systems. Plasmid vectors particularly useful for the transformation and expression of protein in recombinant K. lactis have been descried (Chen, X-J., Gene (1996) 172:131-136). Other yeast expression systems based on Saccharomyces cerevisiae or Pichia pastoris or Pichia methanolica may also be useful for the recombinant production of LEKTI or portions thereof (one or more of domains D1, D2, D3, D4, D5, D6, D7, D8, D9, D10, D11, D12, D13, D14, and D15). Expression plasmid suitable for the expression of LEKTI or portions thereof (one or more of domains D1, D2, D3, D4, D5, D6, D7, D8, D9, D10, D11, D12, D13, D14, and D15) in S. cerevisiae, P. pastoris, or P. methanolica may be obtained from a commercial source or ATCC. Plasmids described above may also be modified by one skilled in the art to optimize, for example, promoter sequences and or secretion signals optimal for the host organism and recombinant production of LEKTI. Established methods are also available to one skilled in the art for introducing recombinant plasmid into the yeast strains.

Expression of recombinant LEKTI or portions thereof (one or more of domains D1, D2, D3, D4, D5, D6, D7, D8, D9, D10, D11, D12, D13, D14, and D15) in mammalian cell culture is also a preferred embodiment of the present invention. There are a wide variety of mammalian cell lines available to one skilled in the art. The most widely used and most successful mammalian expression system is based on a dhfr- (dihydrofolate reductase) Chinese hamster ovary (CHO) cell line along with a suitable expression plasmid containing the dhfr gene and suitable promoter sequence. The cells may be transfected for transient expression or stable expression of the protein of interest. Other factors for the production of foreign protein in mammalian cells including regulatory considerations have been reviewed (Bendig, M., Genetic Engineering (1988) 7:91-127). One useful mammalian expression system is based on the EF-1α promoter (Mizushima, S and Nagata Nucleic Acids Res (1990) 18:5322) and Human embryonic kidney (EK) 293T cell line (Chen, P., et al., Protein Expression and Purification (2002) 24:481-488). Variants of the commercially available CHO and 293T cells lines and their suitable growth and expression media may be used to further improve protein production yields. Variants of commercially available expression vectors including different promoters, secretion signals, transcription enhancers, etc., may also be used to improve protein production yields.

Another useful expression system includes expression in E. coli. There are several expression systems known to one skilled in the art for production of recombinant proteins in E. coli. Expression of mammalian protein in E. coli has not been particularly useful due to the fact that many mammalian proteins are post translationally modified by glycosylation or may contain intra or inter di-sulfide molecular bonds. Particular E. coli expression plasmid useful in the present invention may include, for example, fusions with signal peptides to target the protein to the periplasmic space. Additionally, E. coli host strains that contain mutations in both the thioredoxin reductase (trxB) and glutathione reductase (gor) genes greatly enhance disulfide bond formation in the cytoplasm (Prinz, W. A., et al., J. Biol. Chem. (1997) 272:15661-15667). The addition of thioredoxin fused to the N-terminus or C-terminus of LEKTI may also aid in the production of soluble protein in E. coli cells. (LaVallie, E. R., et al., Bio/Technology (1993) 11:187-193).

Other expression systems known in the art may also be employed for the production of LEKTI or portions thereof (one or more of domains D1, D2, D3, D4, D5, D6, D7, D8, D9, D10, D11, D12, D13, D14, and D15), and include but are not limited to, baculovirus expression (Luckow V., Curr Opin Biotechnol (1993) 5:564-572).

LEKTI or portions thereof (one or more of domains D1, D2, D3, D4, D5, D6, D7, D8, D9, D10, D11, D12, D13, D14, and D15) may be purified from the recombinant expression system using techniques known to one normally skilled in the art. Expression of the LEKTI protein or portions thereof (one or more of domains D1, D2, D3, D4, D5, D6, D7, D8, D9, D10, D11, D12, D13, D14, and D15) can either be intracellular or secreted in the media fraction. Secretion of LEKTI into the media simplifies protein purification. Expression of intracellular LEKTI or portions thereof (one or more of domains D1, D2, D3, D4, D5, D6, D7, D8, D9, D10, D11, D12, D13, D14, and D15) requires disruption of the cell pellets by any convenient method including freeze-thaw, mechanical disruption, sonication, or use of detergents or cell lysing enzymes or agents. Following disruption or concentration of secreted protein, purification can be accomplished by a number of methods know to one skilled in the art. For example, commercially available affinity chromatography may be used to purify recombinant LEKTI or portions thereof (one or more of domains D1, D2, D3, D4, D5, D6, D7, D8, D9, D10, D11, D12, D13, D14, and D15) fused with affinity tags such as: 6×HIS (SEQ ID NO: 120), FLAG® (a convenient binding moiety), GST, or MBP. In addition, antibodies specific to LEKTI or portions thereof (one or more of domains D1, D2, D3, D4, D5, D6, D7, D8, D9, D10, D11, D12, D13, D14, and D15) may be used for affinity purification. In addition, matrices chemically modified with a ligand having strong affinity to LEKTI or portions thereof (one or more of domains D1, D2, D3, D4, D5, D6, D7, D8, D9, D10, D11, D12, D13, D14, and D15) as a substrate mimic may also be used for affinity purification. LEKTI may also be purified with the use of an affinity tag or antibodies following conventional protein purification methods know to one skilled in the art.

According to some embodiments, non-viral gene delivery can also be used. Examples include diffusion of DNA in the absence of any carriers or stabilizers (“naked DNA”), DNA in the presence of pharmacologic stabilizers or carriers (“formulated DNA”), DNA complexed to proteins that facilitate entry into the cell (“Molecular conjugates”), or DNA complexed to lipids.

According to some embodiments, the disclosure provides microbial compositions comprising one or more of a wide range of bacteria. Examples include, but are not limited to, non-pathogenic and commensal bacteria. Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp. According to some embodiments, other related or similar species found on the skin are used.

According to some embodiments, the microbe is engineered to express a mammalian gene encoding LEKTI protein.

Certain embodiments involve the use of bacterium Staphylococcus epidermidis. According to some embodiments, the strain of S. epidermidis to be used is incapable of producing biofilms. An example of this is S. epidermidis strain American Type Culture Collection (ATCC) 12228 or ARS Culture Collection (NRRL) B-4268.

Recent research has shown that the microbiome plays an important role in maintaining and influencing key physiologic activities, including normal metabolism and immune function.

Accordingly, in some embodiments, the disclosure provides microbial compositions comprising a microbe that is genetically modified to express one or more LEKTI protein domains encoded by one or more SPINK genes. According to some embodiments, the one or more SPINK genes encodes one or more LEKTI proteins, or portions thereof (e.g., one or more of domains D1, D2, D3, D4, D5, D6, D7, D8, D9, D10, D11, D12, D13, D14, and D15) that are used to modify the tumor microbiome.

According to some embodiments, the recombinant microbe is adapted to live indefinitely or for a controlled duration on an epithelial or mucosal surface of a mammal to provide a continuous supply of LEKTI protein domains. According to some embodiments, the continuous supply of LEKTI protein domain is provided by constitutively expressed LEKTI. According to some embodiments, the continuous supply of LEKTI protein domain is provided by LEKTI that is inducibly expressed. According to some embodiments, the recombinant microbe lives alongside commensal microorganisms naturally occurring on an epithelial or mucosal surface of the mammal According to some embodiments, the recombinant microbe lives to the exclusion of commensal microorganisms that naturally occur on an epithelial or mucosal surface of the mammal According to some embodiments, the recombinant microbe is adapted to multiply on an epithelial or mucosal surface of the mammal. According to some embodiments, the recombinant microbe is no longer alive, but contains effective amounts of a therapeutic polypeptide, e.g. LEKTI or therapeutically effective domain(s) thereof. Such cells may be intact or not depending upon the particulars of administering the therapeutic peptide (or domain(s) thereof) to the target site.

According to some embodiments, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp. According to some embodiments, the microbe is Staphylococcus epidermidis.

According to some embodiments, the LEKTI protein (or domains thereof) is recombinantly produced and administered. According to some embodiments, the LEKTI protein (or domains thereof) is administered in a composition not including a microbe.

According to some embodiments, the recombinant protein expressed by the engineered microbe comprises the peptide sequence according to SEQ ID NO: 109. In a specific embodiment, the LEKTI domain is Domain 6 (LEKTI D6).

According to some embodiments, the recombinant protein expressed by the engineered microbe comprises a peptide sequence selected from any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 85% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 90% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 95% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 96% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 97% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 98% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 99% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe consists of any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118.

According to some embodiments, the recombinant microbe comprises a sequence as disclosed herein that has at least about 75% identity, 80% identity, 85% identity, 90% identity, 95% identity, 96% identity, 97% identity, 98% identity, or 99% identity to any one or more of the SEQ ID NOS listed herein. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

According to some embodiments, the recombinant protein expressed by the engineered microbe comprises one or more protease inhibitory domains of the LEKTI protein. Some non-limiting examples include one or more of domains D1, D2, D3, D4, D5, D6, D7, D8, D9, D10, D11, D12, D13, D14, and D15. According to some embodiments, the recombinant protein expressed by the engineered microbe comprises LEKTI inhibitory domain 6 (D6) or domains D8 to D11.

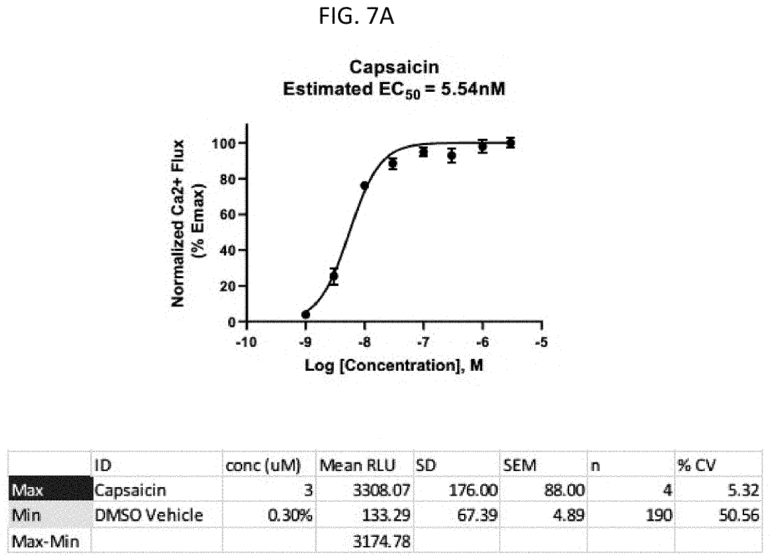

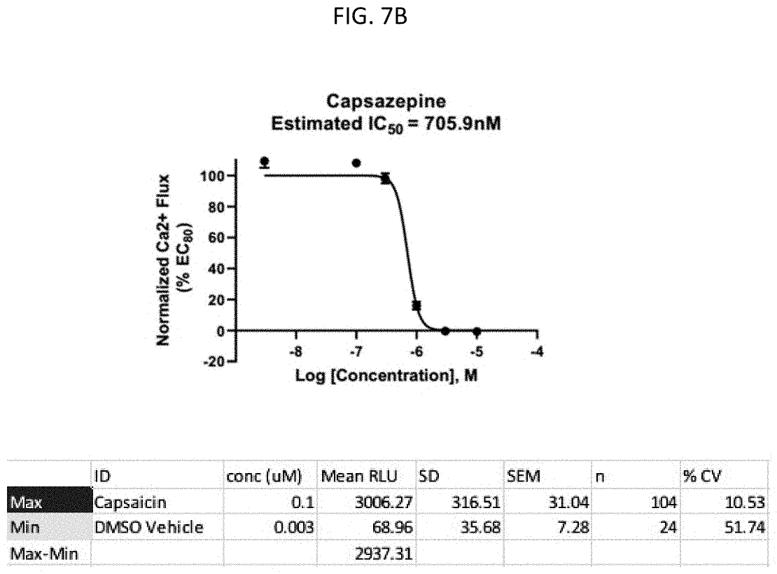

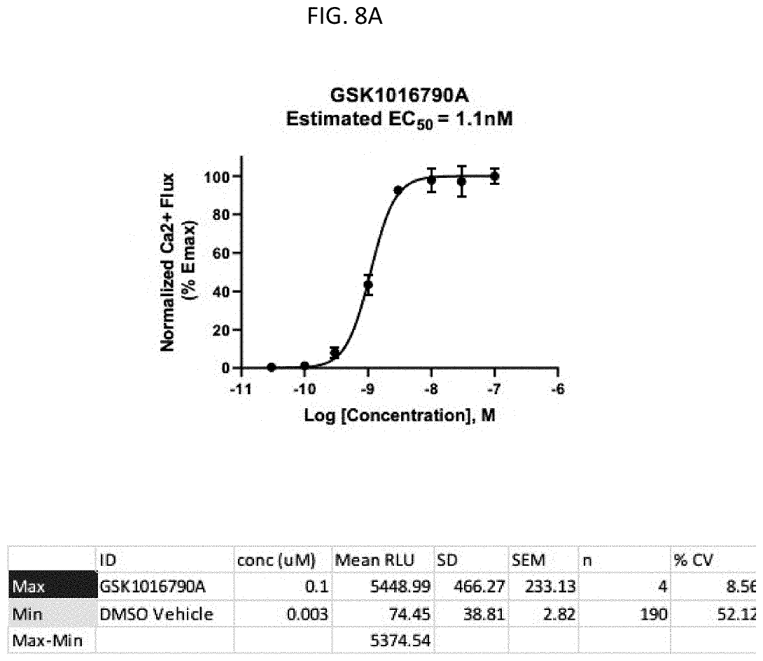

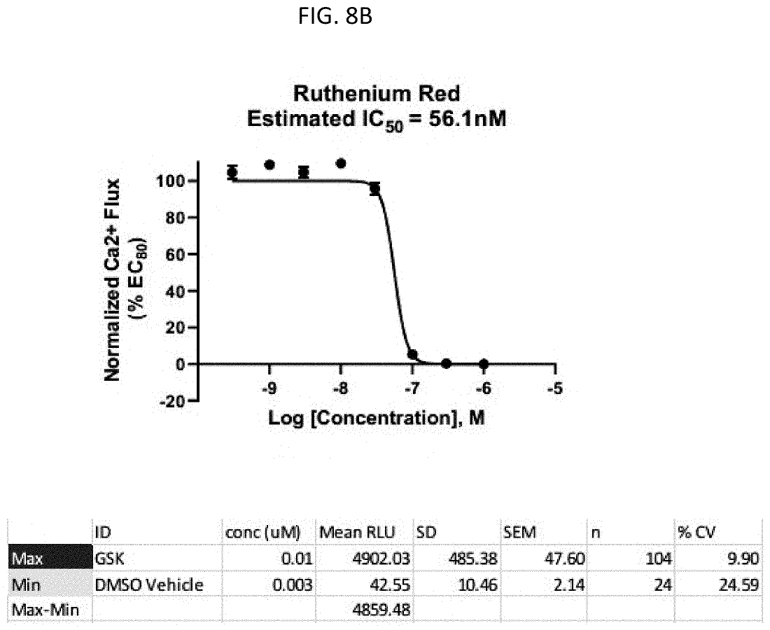

International Patent Application No. PCT/US2018/037850, incorporated by reference in its entirety herein, examines the capacity of purified recombinant LEKTI Domain 6 (LEKTI D6) fragments to function in vitro as a serine protease inhibitor. FIG. 7A and FIG. 7B from PCT/US2018/037850 show recombinantly produced LEKTI Domain 6 inhibits trypsin in vitro. FIG. 8A and FIG. 8B from PCT/US2018/037850 show recombinantly produced LEKTI Domain 6 (with His6 tag (SEQ ID NO: 120)) inhibits trypsin in vitro compared to LEKTI domains 10-15. FIG. 9A and FIG. 9B from PCT/US2018/037850 show recombinantly produced LEKTI Domain 6 inhibits KLK7 in vitro similar to inhibition of KLK7 by LEKTI domains 10-15.

According to some embodiments, the LEKTI protein domains act as a competitive or non-competitive inhibitor of one or more proteases present on or in the skin of a mammal. According to some embodiments, the LEKTI protein domain acts as a serine protease inhibitor.

Secretion Peptides

According to some embodiments, the therapeutic LEKTI domain is operably linked to one or more secretion signals or export signals that tag the protein for transport through the secretory pathway. According to some embodiments, the secretory peptide may be positioned on the N-terminal end of a recombinant protein, and may co-translationally or post-translationally target the tagged protein for secretion. According to some embodiments, at least one LEKTI domain is operably linked to a SecA domain. Any secretion signal that facilitates exit of the LEKTI protein out of the bacterial cell may be used as a secretion peptide. Non-limiting examples of secretion peptides signals are set forth in Table 1, below:

TABLE 1

| Amino Acid Sequence | SEQ ID NO: |
|---|---|
| MKKLAFAITAASGAAAVLSHHDAEA | 9 |
| WLDNRAFSKKFVPVVMATSVALFFLNLAFA | 10 |
| MAKKFNYKLPSMVALTLFGTAFTAHQANA | 11 |
| MKKRFLSICTMTIAALATTTMVNTSYA | 12 |
| NLKKQSKLILIFICIFTFFIMIIQSQFLMG | 13 |
| MKIFKLTSLTLAALTLAFPFSHVAQA | 14 |
| MKKTVIASTLAVSLGIAGYGLSGHEAH | 15 |
| MKKNKFLVYLLSTALITPTFATQTAFA | 16 |
| MKTRQNKYSIRKFSVGASSILIAALLFMGGGSAQA | 17 |
| MKNNNETRRFSIRKYTVGVVSIITGITIFVSGQHAQA | 18 |
| MKKKLSYMITIMLAFTLSLALGLFFNSAHA | 19 |

According to some embodiments, the therapeutic LEKTI domain is operably linked to one or more signal sequences derived from endogenous proteins of Staphylococcus epidermidis. Non-limiting examples of secretion signal peptides derived from endogenous proteins of Staphylococcus epidermidis are set forth in Table 2 below:

TABLE 2

| | Amino | | |
|---|---|---|---|
| | Acid | | SEQ ID |
| Protein Name | Length | Signal Sequence | NO |
| Serine-aspartate repeat- | 45 | MKKRRQGPINKRVDFLSNKVNKYSIRKFTVG | 20 |
| containing protein F | | TASILVGATLMFGA | |
| Glutamyl endopeptidase | 27 | MKKRFLSICTMTIAALATTTMVNTSYA | 21 |
| Bifunctional autolysin | 29 | MAKKFNYKLPSMVALTLFGTAFTAHQANA | 22 |
| Serine-aspartate repeat- | 50 | MIKKNNLLTKKKPIANKSNKYAIRKFTVGTA | 23 |
| containing protein G | | SIVIGAALLFGLGHNEAKA | |
| Biofilm PIA synthesis | 30 | MKPFKLIFISALMILIMTNATPISHLNAQA | 24 |
| deacetylase icaB | | | |
| Lipase | 35 | MKTRQNKYSIRKFSVGASSILIAALLFMGGGS | 25 |
| | | AQA | |
| Epidermin leader | 23 | MNKFKFFIVFLILSLVFLQNEYA | 26 |
| peptide-processing | | | |
| serine protease epiP | | | |
| Fibrinogen-binding | 51 | MINKKNNLLTKKKPIANKSNKYAIRKFTVGT | 27 |
| protein | | ASIVIGATLLFGLGHNEAK | |
| | | A | |
| Staphylococcal | 26 | MKKIATATIATAGIATFAFAHHDAQA | 28 |
| secretory antigen ssaA | | | |
| Extracellular elastase | 28 | MKNFSKFALTSIAALTVASPLVNTEVDA | 29 |
| n/a | 37 | MKNNNETRRFSIRKYTVGVVSIITGITIFVSGQ | 30 |
| | | HAQA | |
| Uncharacterized | 19 | MRYLKRITIYISLLILVSG | 31 |
| lipoprotein SE_0145 | | | |
| Foldase protein prsA | 20 | MKLMNKIIVPVTASALLLGA | 32 |
| Probable cell wall | 40 | MKKIDSWLTKHGLKNRLTLVVIVIFIIFLILLF | 33 |
| amidase lytH | | MFVNLSD | |
| Membrane protein oxaA | 19 | MKKKALLPLFLGIMIFLAG | 34 |
| 2 | | | |
| Probable | 28 | MKKTVIASTLAVSLGIAGYGLSGHEAHA | 35 |
| transglycosylase isaA | | | |
| Probable quinol oxidase | 19 | MSKFKSLLLLFGTLILLSG | 36 |
| subunit 2 | | | |
| Probable | 27 | MKKTLVASSLAIGLGVVAGNAGHDAHA | 37 |
| transglycosylase sceD | | | |
| Bifunctional autolysin | 29 | MAKKFNYKLPSMVALTLFGTAFTAHQANA | 38 |
| Extracellular cysteine | 30 | MKKKLSYMITIMLAFTLSLALGLFFNSAHA | 39 |
| protease | | | |
| Membrane protein oxaA | 18 | MHKRLFITLLGFIILLAG | 40 |
| 1 | | | |
| Uncharacterized | 19 | MRYLKRITIYISLLILVSG | 41 |
| lipoprotein SE_0144 | | | |
| N-acetylmuramoyl-L- | 25 | MQKKYITAIIGTTALSALASTHAQA | 42 |
| alanine amidase slel | | | |
| Uncharacterized | 22 | MKHSSKIIVFVSFLILTIFIGG | 43 |
| lipoprotein SE_0142 | | | |
| Phosphate-binding | 20 | MKKWQLVGTTVLGASVLLGA | 44 |
| protein pstS | | | |
| Accumulation- | 52 | MGKRRQGPINKKVDFLPNKLNKYSIRKFTVG | 45 |
| associated protein | | TASILLGSTLIFGSSSHEAKA | |
| Staphylococcal | 26 | MKKIATATIATAGIATFAFAHHDAQA | 46 |
| secretory antigen ssaA | | | |
| Serine-aspartate repeat- | 45 | MKKRRQGPINKRVDFLSNKVNKYSIRKFTVG | 47 |
| containing protein F | | TASILVGATLMFGA | |
| Glutamyl endopeptidase | 27 | MKKRFLSICTMTIAALATTTMVNTSYA | 48 |
| Lipase | 35 | MKTRQNKYSIRKFSVGASSILIAALLFMGGGS | 49 |
| | | AQA | |
| Extracellular elastase | 28 | MKNFSKFALTSIAALTVASPLVNTEVDA | 50 |
| Uncharacterized | 17 | MKKVLASATILSLMLVG | 51 |
| lipoprotein SE_1947 | | | |
| Uncharacterized | 22 | MKYYGKCISYISILILTFFIGG | 52 |
| lipoprotein | | | |
| SE_0186/SE_0187 | | | |
| Uncharacterized | 22 | MKHSSKIIVFVSFLILTIFIGG | 53 |
| lipoprotein SERP2423 | | | |
| Biofilm PIA synthesis | 30 | MKPFKLIFISALMILIMTNATPISHLNAQA | 54 |
| deacetylase icaB | | | |
| Probable quinol oxidase | 19 | MSKFKSLLLLFGTLILLSG | 55 |
| subunit 2 | | | |
| Probable | 27 | MKKTLVASSLAIGLGVVAGNAGHDAHA | 56 |
| transglycosylase sceD | | | |
| Uncharacterized | 19 | MHYLKKVTIYISLLILVSG | 57 |
| lipoprotein SERP2447 | | | |
| N-acetylmuramoyl-L- | 25 | MQKKYITAIIGTTALSALASTHAQA | 58 |
| alanine amidase slel | | | |
| Uncharacterized | 22 | MKHSKKLLLCISFLLITFFIGG | 59 |
| lipoprotein SERP2445 | | | |
| Staphylococcal | 26 | MKKIATATIATAGIATFAFAHHDAQA | 60 |
| secretory antigen ssaA | | | |
| Uncharacterized | 19 | MRYLKKVTIYISLLILVSG | 61 |
| lipoprotein SERP2443 | | | |
| Glutamyl endopeptidase | 27 | MKKRFLSICTMTIAALATTTMVNTSYA | 62 |
| Phosphate-binding | 20 | MKKWQLVGTTVLGASVLLGA | 63 |
| protein pstS | | | |
| Bifunctional autolysin | 29 | MAKKFNYKLPSMVALTLFGTAFTAHQANA | 64 |
| Extracellular cysteine | 30 | MKKKLSYMITIMLAFTLSLALGLFFNSAHA | 65 |
| protease | | | |
| Membrane protein oxaA | 18 | MHKRLFITLLGFIILLAG | 66 |
| 1 | | | |
| Uncharacterized | 22 | MRYLKKVTIYISLLILTIFIGG | 67 |
| lipoprotein SERP2422 | | | |
| Uncharacterized | 17 | MKKVLASATILSLMLVG | 68 |
| lipoprotein SERP 1959 | | | |
| Uncharacterized | 22 | MKHSKKLLLCISFLLITVFISG | 69 |
| lipoprotein SERP2453 | | | |
| Uncharacterized | 22 | MKHSKKLLLCISFLLITFFISG | 70 |
| lipoprotein SERP2465 | | | |
| Probable | 28 | MKKTVIASTLAVSLGIAGYGLSGHEAHA | 71 |
| transglycosylase isaA | | | |
| Uncharacterized | 22 | MKHSKKLLLCISFLLITIFISG | 72 |
| lipoprotein SERP2451 | | | |
| Probable cell wall | 40 | MKKIDSWLTKHGLKNRLTLVVIVIFIIFLILLF | 73 |
| amidase lytH | | MFVNLSD | |
| Membrane protein oxaA | 19 | MKKKALLPLFLGIMIFLAG | 74 |
| 2 | | | |
| Foldase protein prsA | 20 | MKLMNKIIVPVTASALLLGA | 75 |
| Lipase | 35 | MKTRQNKYSIRKFSVGASSILIAALLFMGGGS | 76 |
| | | AQA | |

According to some embodiments, the therapeutic LEKTI domain is operably linked to one or more secretion signal sequences derived from endogenous proteins of other bacteria. Signal peptides derived from endogenous proteins of various bacteria are known in the art.

Cell Penetration Peptides

According to some embodiments, one or more cell penetrating peptides are used to mediate delivery of therapeutic proteins in vivo without using cell surface receptors and without causing significant membrane damage. According to some embodiments, the recombinant LEKTI domain is operably linked to a cell penetration peptide sequence that enhances the ability of the LEKTI domain to pass through a cell membrane. The term “enhance” as used to describe the cell penetration peptide/LEKTI, means that the cell penetration sequence improves the passage of recombinant LEKTI domain through a cell membrane relative to a recombinant LEKTI domain lacking the cell penetration sequence.

According to some embodiments, one or more cell penetrating peptides are operably linked to therapeutic proteins to facilitate entry into skin cells (e.g. keratinocytes). Non-limiting examples are set forth in Table 3, below:

TABLE 3

| Cell penetrating sequence | SEO ID NO |
|---|---|
| GRKKRRQRRRPPQ | 77 |
| GWTLNS AGYLLGKINLKALAALAKKIL | 78 |
| KLALKLALKALKAALKLA | 79 |
| WEAKLAKALAKALAKHLAKALAKALKACEA | 80 |
| KETWWETWWTEWSQPKKKRKV | 81 |
| RRRRRRRRR | 82 |
| LGTYTQDFNKFHTFPQTAIGVGAP | 83 |
| RQIKWFQNRRMKWKK | 84 |
| YGRKKRRQRRR | 85 |
| RGGRLSYSRRRFSTSTGR | 86 |
| RRLSYSRRRF | 87 |
| PIRRRKKLRRLK | 88 |
| RRQRRTSKLMKR | 89 |
| RRRRNRTRRNRRRVR | 90 |
| KMTRAQRRAAARRNRWTAR | 91 |
| TRRQRTRRARRNR | 92 |
| GRKKRRQRRRPPQ | 93 |
| GRRRRRRRRRPPQ | 94 |
| GWTLNSAGYLLGKINLKALAALAKKIL | 95 |
| KLALKLALKLALALKLA | 96 |
| MGLGLHLLVLAAALQGAWSQPKKKRKV | 97 |
| GALFLGWLGAAGSTMGAWSQPKKKRKV | 98 |
| GALFLGFLGAAGSTMGAWSQPKKKRKV | 99 |
| GALFLGFLGAAGSTMGAWSQPKSKRKV | 100 |
| KETWWETWWTEWSQPKKKRKV | 101 |
| KETWFETWFTEWSQPKKKRKV | 102 |

According to some embodiments, cell penetrating peptides comprise periodic amino acid sequences. Non-limiting examples of periodic cell penetrating sequences include: Polyarginines, R×n (wherein 4<n<17) (SEQ ID NO: 126); Polylysines, K×n (wherein 4<n<17) (SEQ ID NO: 127); arginine repeats interspaced with 6-aminocaprotic acid residues (RAca), wherein there are 2 to 6 arginine repeats; arginine repeats interspaced with 4-aminobutyric acid (RAbu), wherein there are 2 to 6 arginine repeats; arginine repeats interspaced with methionine, wherein there are 2 to 6 arginine repeats; arginine repeats interspaced with threonine, wherein there are 2 to 6 arginine repeats; arginine repeats interspaced with serine, wherein there are 2 to 6 arginine repeats; and arginine repeats interspaced with alanine, wherein there are 2 to 6 arginine repeats.

According to some embodiments, the LEKTI domain is operably linked to an RMR domain.

According to some embodiments, expression of the LEKTI domain is controlled by an operon and the amount of LEKTI provided to the mammal's skin is proportional to the availability of an extrinsic factor. For example, according to some embodiments the recombinant LEKTI gene may be under the control of a xylose inducible promoter (e.g. xylose repressor (xylR), xylose operator (xylO), xylose isomerase gene (xylA) including the cis-acting catabolite-responsive element (CRE)), and the amount of recombinant LEKTI protein made available to the skin of the mammal controlled by the amount of exogenous xylose available to the recombinant microbe. According to some embodiments, the expression of the LEKTI domain is controlled by a promoter that is constitutively active. According to some embodiments, the expression of the LEKTI domain is controlled by a Cm^{R} promoter.

According to some embodiments, the microbe is genetically modified by transfection/transformation with a recombinant DNA plasmid encoding one or more of the LEKTI protein domains and one or more antibiotic resistance genes. For example, some embodiments of the recombinant DNA plasmid comprise a kanamycin resistance gene and/or a trimethoprim resistance gene; e.g. dfrA. According to some embodiments, treatment of the skin of the mammal with an antibiotic (for which the recombinant microbe is resistant) may be used to bias the population of commensal microbes toward a larger proportion of LEKTI producing microbes. Other elements that may be present in the recombinant DNA plasmid include, without limitation, a replication protein gene, such as a member of the Rep superfamily of replication proteins. For example, according to some embodiments the recombinant DNA plasmid comprises the repF gene.

According to some embodiments, the recombinant DNA plasmid comprises one or more sequences of the pJB38 vector. According to some embodiments, the recombinant LEKTI is operably linked to an inducible promoter, ribosome binding site, export signal, and/or cell penetrating peptide in the pJB38 vector. As used herein, the term “pJB38-LEKTI-complete” means a recombinant DNA plasmid construct comprising the pJB38 vector and one or more LEKTI domains.

According to some embodiments, the recombinant DNA plasmid comprises the pKK30-LEKTI as shown in FIG. 1.

According to some such embodiments, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp.

According to some embodiments, the amount or durations of availability of therapeutic LEKTI protein is controlled by the stability of the vector harboring the LEKTI in a microbe. For example, the persistence of a recombinant vector may be controlled by one or more elements of a plasmid including those that provide host-beneficial genes, plasmid stability mechanisms, and plasmid co-adaptation. For example, some plasmid may provide for stable replication, active partitioning mechanisms, and mechanisms that insure reliable inheritance of plasmids to daughter cells over generations. (See, e.g., J. C. Baxter, B. E. Funnell, Plasmid partition mechanisms, Microbiol. Spectr., 2 (2014) PLAS-0023-2014 and Nils Wilier et al., An evolutionary perspective on plasmid lifestyle modes, Current Opinion in Microbiology, Volume 38, August 2017, Pages 74-80, each of which are incorporated by herein by reference in its entirety) According to some embodiments, the present invention includes the use of all conventional selection and stability methods known to a person of skill in the art.

### 3. Vector Encoded LEKTI Domains

LEKTI nucleic acids may be expressed from transcription units inserted into DNA or RNA vectors (see, e.g., Couture, A, et al., TIG. (1996), 12:5-10; WO 00/22113, WO 00/22114, and U.S. Pat. No. 6,054,299). According some embodiments, a LEKTI nucleic acid is comprised in a vector, such as a viral expression vector. According some embodiments, the LEKTI nucleic acid comprises SEQ ID NO: 119, or a fragment thereof. According to some embodiments, the LEKTI nucleic acid is at least 75%, at least 80%, at least 85%, at least 90%, at least 95%, at least 96%, at least 97%, at least 98% or at least 99% identical to SEQ ID NO: 119.

In some embodiment, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

Delivery of a LEKTI (e.g., LEKTI D6) expressing vector can be systemic, such as by intravenous or intramuscular administration, by administration to target cells ex-planted from the patient followed by reintroduction into the patient, or by any other means that allows for introduction into a desired target cell.

In certain embodiment, the nucleic acids described herein or the nucleic acids encoding a protein described herein, e.g., an effector, are incorporated into a vector, e.g., a viral vector.

The individual strand or strands of a LEKTI (e.g., LEKTI D6) nucleic acid molecule can be transcribed from a promoter in an expression vector. Where two separate strands are to be expressed to generate, for example, a dsRNA, two separate expression vectors can be co-introduced (e.g., by transfection or infection) into a target cell. Alternatively, each individual strand of a nucleic acid molecule can be transcribed by promoters both of which are located on the same expression plasmid. In one embodiment, a nucleic acid molecule is expressed as inverted repeat polynucleotides joined by a linker polynucleotide sequence such that the nucleic acid molecule has a stem and loop structure.

Expression vectors are generally DNA plasmids or viral vectors. Expression vectors compatible with eukaryotic cells, preferably those compatible with vertebrate cells, can be used to produce recombinant constructs for the expression of LEKTI (e.g., LEKTI D6) as described herein.

Constructs for the recombinant expression of LEKTI (e.g., LEKTI D6) will generally require regulatory elements, e.g., promoters, enhancers, etc., to ensure the expression of LEKTI (e.g., LEKTI D6) in target cells.

Expression of natural or synthetic nucleic acids is typically achieved by operably linking a nucleic acid encoding the nucleic acid of interest to a regulatory region, such as a promoter, and incorporating the construct into an expression vector. The vectors can be suitable for replication and integration in eukaryotes.

Regulatory regions, such as a promoter, suitable for operable linking to a nucleic acid molecules can be operably linked to a regulatory region such as a promoter. can be from any species. Any type of promoter can be operably linked to a nucleic acid sequence. Examples of promoters include, without limitation, tissue-specific promoters, constitutive promoters, and promoters responsive or unresponsive to a particular stimulus (e.g., inducible promoters). Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Additional regulatory regions that may be useful in nucleic acid constructs, include, but are not limited to, transcription and translation terminators, initiation sequences, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like. Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

Signal peptides may also be included and can be used such that an encoded polypeptide is directed to a particular cellular location (e.g., the cell surface).

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Other aspects to consider for vectors and constructs are known in the art.

In some embodiments, a vector, e.g., a viral vector comprises a LEKTI (e.g., LEKTI D6) comprising a site-specific LEKTI (e.g., LEKTI D6) targeting moiety comprising a nucleic acid molecule.

Viral vector systems which can be utilized with the methods and compositions described herein include, but are not limited to, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein.

Vectors, including those derived from retroviruses such as adenoviruses and adeno-associated viruses and lentiviruses, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals.

In one embodiment, a suitable viral vector for use in the present invention is an adeno-associated viral vector, such as a recombinant adeno-associate viral vector.

Recombinant adeno-associated virus vectors (rAAV) are gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

### 4. Pharmaceutical Compositions

Aspects of the present disclosure include one or more of the LEKTI protein domains described herein, in combination with a pharmaceutically acceptable carrier. The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety.

A pharmaceutical composition is formulated to be compatible with its intended route of administration. Pharmaceutical compositions as described herein may be administered to a mammalian host in a variety of forms adapted to the chosen route of administration, e.g., orally or parenterally. Parenteral administration includes administration by the following routes: intramuscular; subcutaneous; intraocular; intrasynovial; transepithelial including transdermal, ophthalmic, sublingual and buccal; topically, including ophthalmic, dermal, ocular, and rectal; and nasal inhalation via insufflations and aerosols, including nasopharyngeal and throat installation.

Solutions or suspensions used for parenteral application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include aqueous solutions (where water soluble) or dispersions and powders for the extemporaneous preparation of injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

According to some embodiments, the compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

According to some embodiments the compositions comprising a microbe genetically modified to express one or more LEKTI protein domains encoded by one or more SPINK genes to for use according to the present invention can comprise any pharmaceutically effective amount of the recombinant bacteria to produce a therapeutically effective amount of the desired polypeptide or therapeutically effective domain(s) thereof, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about. 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of recombinant bacteria, the upper limit of which is about 90.0% by weight of recombinant, bacteria.

According to some embodiments, the composition for use according to the present invention can comprise, for example, at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, or more by weight of recombinant bacteria.

According to some embodiments, the composition for use according to the disclosure is a cosmetic formulation.

According to some embodiments, the composition is a topical formulation. According to some embodiments, the topical formulation can be in any form suitable for application to the body surface, such as a cream, lotion, sprays, solution, gel, ointment, paste, plaster, paint, bioadhesive, suspensions, emulsions, or the like, and/or can be prepared so as to contain liposomes, microsomes, micelles, and/or microspheres. Such a formulation can be used in combination with an occlusive overlayer so that moisture evaporating from the body surface is maintained within the formulation upon application to the body surface and thereafter.

According to some embodiments, the formulation can include a living cell culture composition and can comprise at least one engineered bacterial strain that produces a therapeutically effective recombinant polypeptide or therapeutically effective domain(s) thereof. According to some embodiments, this engineered living cell culture composition can deliver the polypeptide directly to the skin for treating or preventing abnormal skin conditions.

Topical formulations include those in which any other active ingredient(s) is (are) dissolved or dispersed in a dermatological vehicle known in the art (e.g. aqueous or nonaqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed can contain one or more components (for example, when the formulation is an aqueous gel, components in addition to water) selected from the following list: a solubilizing agent or solvent (e.g. a β-cyclodextrin, such as bydroxypropyl β-cyclodextrin, or an alcohol or polyol such as ethanol, propylene glycol or glycerol); a thickening agent (e.g. hydroxyethylceliulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

According to some embodiments, the pharmaceutical composition of the invention can be applied in combination with (solid) carriers or matrices such as dressing(s), band aid(s) or tape(s). The compound(s) can be covalently or non-covalently bound to said carrier or matrix.

For example, the compound(s) may be incorporated into a dressing to be applied over a lesion. Examples of such dressings include staged or layered dressings incorporating slow-release hydrocolloid particles containing the composition or sponges containing the wound healing material optionally covered by conventional dressings. The concentration of a solution of the pharmaceutical composition to be immobilized in a matrix of a wound dressing is generally in the range of 0.001 to 1% (w/v) preferably 0.01-0.1% (w/v). Furthermore, the compound(s) as recited above can be incorporated into a suitable material capable of delivering the enzyme to a wound in a slow release or controlled release manner.

According to some embodiments, a topical formulation may also include a skin lightening agent. Suitable skin lightening agents include, but are not limited to, ascorbic acid and derivatives thereof; kojic acid and derivatives thereof; hydroquinone; azelaic acid; and various plant extracts, such as those from licorice, grape seed, and bear berry. A skin conditioning agent includes, for example, a substance that enhances the appearance of dry or damaged skin, as well as a material that adheres to the skin to reduce flaking, restore suppleness, and generally improve the appearance of skin. Representative examples of a skin conditioning agent that may be used include: acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, amyloglucosidase, arbutin, arginine, bromelain, buttermilk powder, butylene glycol, calcium gluconate, carbocysteine, carnosine, beta-carotene, casein, catalase, cephalins, ceramides, Chamomilla recutita (matricaria) flower extract, cholecalciferol, cholesteryl esters, coco-betaine, corn starch modified, cry stallins, cycloethoxymethicone, cysteine DNA, cytochrome C, darutoside, dextran sulfate, dimethicone copolyols, dimethylsilanol hyaluronate, elastin, elastin amino acids, ergocalciferol, ergosterol, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin. Other non-limiting examples of a skin conditioning agent that may be included in the compositions includes lactoferrin, lanosterol, lecithin, linoleic acid, linolenic acid, lipase, lysine, lysozyme, malt extract, maltodextrin, melanin, methionine, niacin, niacinamide, oat amino acids, oryzanol, palmitoyl hydrolyzed proteins, pancreatin, papain, polyethylene glycol, pepsin, phospholipids, phytosterols, placental enzymes, placental lipids, pyridoxal 5-phosphate, quercetin, resorcinol acetate, riboflavin, saccharomyces lysate extract, silk amino acids, sphingolipids, stearamidopropyl betaine, stearyl palmitate, tocopherol, tocopheryl acetate, tocopheryl linoleate, ubiquinone, Vitis vinifera (grape) seed oil, wheat amino acids, xanthan gum, and zinc gluconate. Skin protectant agents include, for example, a compound that protects injured or exposed skin or mucous membrane surfaces from harmful or irritating external compounds. Representative examples include algae extract, allantoin, aluminum hydroxide, aluminum sulfate, Camellia sinensis leaf extract, cerebrosides, dimethicone, glucuronolactone, glycerin, kaolin, lanolin, malt extract, mineral oil, petrolatum, potassium gluconate, and talc.

An emollient may be included in a pharmaceutical or cosmetic composition of the disclosure. An emollient generally refers to a cosmetic ingredient that can help skin maintain a soft, smooth, and pliable appearance. Emollients typically remain on the skin surface, or in the stratum corneum, to act as a lubricant and reduce flaking. Some examples of an emollient include acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, glycol esters, alkyl lactates, caprylyl glycol, cetyl esters, cetyl laurate, coconut oil polyethylene glycol-10 esters, alkyl tartrates, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, Odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, and tocopheryl glucoside.

Humectants are cosmetic ingredients that help maintain moisture levels in skin. Examples of humectants include acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, sodium pyrollidone carboxylic acid (PCA), sorbitol, sucrose, and urea.

A pharmaceutically acceptable carrier can also be incorporated in the compositions of the present invention and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation, “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

A film former, when it dries, forms a protective film over the site of application. The film inhibits removal of the active ingredient and keeps it in contact with the site being treated. An example of a film former that is suitable for use in this invention is Flexible Collodion, US P. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the ease of applying a more fluid composition. It is generally necessary that the insoluble matter in a lotion be finely-divided.

Lotions will typically contain suspending agents to produce better dispersions as well as compounds useful for localizing and holding the active agent in contact with the skin, e.g., methylcellulose, sodium earhoxymethyl-celiulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred “organic macromolecules,” i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthaiate, and methylcellulose; gums such as tragacanth and xanthan gum; sodium alginate; and gelatin, In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter δ of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present invention in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in the formulations of the invention are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), α-tocopherol (Vitamin E), β-tocopherol, γ-tocopherol, δ-tocopherol, ε-tocopherol, ζι-tocopherol, Z^{∧}-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), α-tocopherol (Vitamin E), β-tocopherol, γ-tocopherol, δ-tocopherol, ε-tocopherol, ζι-tocopherol, ζ₂-tocopherol, η-tocopherol, and retinol (Vitamin A), and/or pharmaceutically acceptable salts, esters, amides, or other derivatives thereof. A preferred tocopherol compound is α-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene. See Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from about 0.01% to about 20% of the total weight of the topical formulation.

The compositions of the invention can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds.

Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. In other embodiments, other agents can also be added, such as repressors and inducers, i.e., to inhibit (i.e. glycose) or induce (i.e. xylose) the production of the polypeptide of interest. Such additives can be employed provided they are compatible with and do not interfere with the function of the formulations.

According to some embodiments, the compositions can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the invention will be applied in the range of once or twice weekly up to once or twice daily.

The pharmaceutical compositions of the invention comprise one or more active ingredients, e.g. therapeutic agents, in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Dosage forms for the topical or transdermal administration include powders, sprays, ointments, pastes, creams, lotions, gels, solutions, patches, drops and inhalants. The active agent(s)/compound(s) may be mixed with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable isotonic aqueous or non-aqueous solutions, dispersions, suspensions or emulsions, or powders which may be reconstituted into injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

### 5. Methods of Treatment

The present disclosure is based in part on the finding that the administration of one or more LEKTI protein domains can be used to treat or prevent skin diseases or disorders. According to some embodiments, the skin disease or disorder is an inflammatory skin disease or disorder. According to some embodiments, the skin disease or disorder is pruritus. According to some embodiments, the skin disease or disorder is pain.

It is to be understood that when any of the methods described herein comprise administering a microbe genetically modified to express one or more LEKTI protein domains encoded by one or more SPINK genes, the one or more LEKTI protein domains may penetrate the skin in combination with the microbe or separately from the microbe.

### Inflammatory Skin Diseases or Disorders

According to aspects of the present disclosure, administration of one or more LEKTI protein domains can be used to decrease the skin inflammatory response. As such, the methods of the present disclosure can be used to treat or prevent one or more symptoms of an inflammatory skin disease or disorder. The inflammatory response is an important component of the immune system. However, the inflammatory response can destroy healthy tissue and cause tissue damage. In the case of inflammatory skin disorders, subjects may experience short term or long term symptoms including swelling, redness, a rash or hives, pustules, dryness, itching, and burst capillaries. Exemplary inflammatory skin diseases or disorders include, but are not limited to, rosacea, psoriasis and atopic dermatitis. Further exemplary inflammatory skin diseases or disorders include, but are not limited to, acne, seborrheic dermatitis, contact dermatitis, boils, carbuncles, pemphigus, cellulitis, Grover's disease, hidradenitis suppurativa, or lichen planus. Accordingly, the invention provides methods for treating (decreasing or ameliorating one or more symptoms of) acne, rosacea, psoriasis, atopic dermatitis, seborrheic dermatitis, contact dermatitis, boils, carbuncles, pemphigus, cellulitis, Grover's disease, hidradenitis suppurativa, and lichen planus.

The present disclosure provides a method of treating an inflammatory skin disease or disorder in a subject in need thereof, comprising administering one or more LEKTI protein domains to a subject to provide a therapeutic effect to decrease symptoms of the inflammatory skin disorder.

The present disclosure provides a method of treating an inflammatory skin disease or disorder in a subject in need thereof, comprising administering a microbe genetically modified to express one or more LEKTI protein domains encoded by one or more SPINK genes to a subject to provide a therapeutic effect to decrease symptoms of the inflammatory skin disorder.

The present disclosure provides a method of preventing an inflammatory skin disease or disorder in a subject in need thereof, comprising administering one or more LEKTI protein domains to provide a therapeutic effect to prevent symptoms of the inflammatory skin disorder.

The present disclosure provides a method of preventing an inflammatory skin disease or disorder in a subject in need thereof, comprising administering a microbe genetically modified to express one or more LEKTI protein domains encoded by one or more SPINK genes to provide a therapeutic effect to prevent symptoms of the inflammatory skin disorder.

According to some embodiments, preventing an inflammatory skin disorder is intended to encompass preventing the progression of an inflammatory skin disorder (e.g., preventing the progression of symptoms of the inflammatory skin disease or disorder).

The present disclosure provides a method of treating an inflammatory skin disease or disorder in a subject in need thereof, comprising administering one or more LEKTI protein domains to the skin of a subject in need thereof, wherein the microbe genetically modified to express one or more LEKTI protein domains encoded by one or more SPINK genes penetrates the skin to provide a therapeutic effect to decrease symptoms of the inflammatory skin disorder.

The present disclosure provides a method of treating an inflammatory skin disease or disorder in a subject in need thereof, comprising administering a microbe genetically modified to express one or more LEKTI protein domains encoded by one or more SPINK genes to the skin of a subject in need thereof, wherein the microbe genetically modified to express one or more LEKTI protein domains encoded by one or more SPINK genes penetrates the skin to provide a therapeutic effect to decrease symptoms of the inflammatory skin disorder.

The present disclosure provides a method of preventing an inflammatory skin disease or disorder in a subject in need thereof, comprising administering one or more LEKTI protein domains to the skin of a subject in need thereof, wherein the microbe genetically modified to express one or more LEKTI protein domains encoded by one or more SPINK genes penetrates the skin to provide a therapeutic effect to prevent symptoms of the inflammatory skin disorder.

The present disclosure provides a method of preventing an inflammatory skin disease or disorder in a subject in need thereof, comprising administering a microbe genetically modified to express one or more LEKTI protein domains encoded by one or more SPINK genes to the skin of a subject in need thereof, wherein the microbe genetically modified to express one or more LEKTI protein domains encoded by one or more SPINK genes penetrates the skin to provide a therapeutic effect to prevent symptoms of the inflammatory skin disorder. According to some embodiments, preventing an inflammatory skin disorder is intended to encompass preventing the progression of an inflammatory skin disorder (e.g., preventing the progression of symptoms of the inflammatory skin disease or disorder).

According to some embodiments, the methods of treating or preventing an inflammatory skin disease or disorder in a subject as described herein results in an increase in the induction of an immune response.

According to some embodiments, the immune response is an innate immune response. The innate arm of the immune system is a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs). When a pathogen breaches the initial barriers of the skin or a mucosal surface, both soluble and cellular innate defense mechanisms are encountered and an inflammatory response is rapidly initiated. Soluble inflammatory chemokines and activated complement produced in response to pathogen sensing contribute to the attraction of additional innate immune cells such as neutrophils, NK cells, and monocytes to the site of infection. The recruited inflammatory cells encircle the damaged or infected cells and release more proinflammatory cytokines including tumor necrosis factor (TNF), IL-6, IL-12, and type I and II interferons (IFNs).

According to some aspects, the present disclosure provides a method for decreasing the number of skin lesions in a subject suffering from an inflammatory skin disease or disorder, comprising administering one or more LEKTI protein domains to provide a therapeutic effect to decrease the number of skin lesions.

According to some aspects, the present disclosure provides a method for decreasing the number of skin lesions in a subject suffering from an inflammatory skin disease or disorder, comprising administering a microbe genetically modified to express one or more LEKTI protein domains encoded by one or more SPINK genes to provide a therapeutic effect to decrease the number of skin lesions.

According to some aspects, the present disclosure provides a method for decreasing the number of skin lesions in a subject suffering from an inflammatory skin disease or disorder, comprising administering a microbe genetically modified to express one or more LEKTI protein domains encoded by one or more SPINK genes to the skin of a subject in need thereof, wherein the microbe genetically modified to express one or more LEKTI protein domains encoded by one or more SPINK genes penetrates the skin to provide a therapeutic effect to decrease the number of skin lesions.

According to some aspect, the present disclosure provides a method for decreasing the number of skin lesions in a subject suffering from an inflammatory skin disease or disorder, comprising administering a microbe genetically modified to express one or more LEKTI protein domains encoded by one or more SPINK genes to the skin of a subject in need thereof, wherein the microbe genetically modified to express one or more LEKTI protein domains encoded by one or more SPINK genes penetrates the skin to provide a therapeutic effect to decrease the number of skin lesions.

According to some embodiments, the one or more symptoms of the inflammatory skin disease or disorder that are treated are selected from one or more of expression of an inflammatory cytokine, inflammation, pain, itching, skin dryness, skin flaking, bacterial count, number of skin lesions, severity of skin lesions, frequency of outbreaks of skin lesions, redness, and skin discoloration

According to some embodiments, the inflammatory skin disorder is rosacea. In such embodiments, “treating” rosacea includes decreasing the severity, frequency, and/or occurrence of one or more of the symptoms of rosacea. According to some embodiments, the inflammatory skin disorder is psoriasis. In such embodiments, “treating” psoriasis includes decreasing the severity, frequency, and/or occurrence of any one or more of the symptoms of psoriasis. According to some embodiments, the inflammatory skin disorder is atopic dermatitis. In such embodiments, “treating” atopic dermatitis includes decreasing the severity, frequency, and/or occurrence of any one or more of the symptoms of atopic dermatitis.

According to some embodiments, administration of one or more LEKTI protein domains to the subject decreases the symptoms of the inflammatory skin disorder and promotes healing of the affected skin tissue without significant scarring.

According to some embodiments, administration of a microbe genetically modified to express one or more LEKTI protein domains encoded by one or more SPINK genes to the subject decreases the symptoms of the inflammatory skin disorder and promotes healing of the effected skin tissue without significant scarring.

According to some embodiments, the therapeutic effect includes decreasing an inflammatory response, as assayed by expression of TNF-α or other inflammatory cytokine. Therapeutic efficacy also includes one or more of decreasing bacterial count, increasing healing, and increasing proliferation of healthy skin tissue. Over the course of therapy, therapeutic efficacy can be assessed by evaluating decrease in the presence or severity of the symptoms of the inflammatory skin disorder.

According to some embodiments, the method for treating or preventing an inflammatory skin disorder comprises multiple treatments (e.g., 1, 2, 3, 4, 5, 6, 7, 8, 9, 10, 15, 20, 25, 20, 35, 40, 45, 50, 55, 60, 65, 70, 75, 80, 85, 90, 95, 100 or more treatments).

The appropriate number of treatments, and the duration of each treatment, can be determined by a health care provider based on, for example, the particular inflammatory skin disorder being treated, the severity of the disorder, and the overall health of the subject.

According to some embodiments, the method is part of a therapeutic regimen combining one or more additional treatment modalities as part of a therapeutic regimen for treating an inflammatory skin disorder.

When used in this way, administration of a microbe genetically modified to express one or more LEKTI protein domains encoded by one or more SPINK genes can act additively or synergistically with other treatments. Exemplary therapies include, but are not limited to, antibiotics, hydrocortisone creams, benzoil peroxide, retinoids and other vitamin A based agents, steroids or other immunosuppressive agents (methotrexate, cyclosporin), and the like. Further exemplary therapies include cytokine antagonists, such as TNF-α antagonists designed to decrease expression of TNF-α. Further exemplary therapies include phototherapy, a specialized dietary regimen, acupuncture, stress management, exercise, and the like.

Rosacea

Rosacea is a common facial dermatitis that currently affects an estimated 13 million Americans. It begins as erythema (flushing and redness) on the central face and across the cheeks, nose, or forehead but can also less commonly affect the neck and chest. As rosacea progresses, other symptoms can develop such as semi-permanent erythema, telangiectasia (dilation of superficial blood vessels on the face), red bumps and pustules, red gritty eyes, burning and stinging sensations, and in some advanced cases, rhinophyma.

There are four identified rosacea subtypes and patients may have symptoms characteristic of more than one subtype.

1. Erythematotelangiectatic rosacea: This subtype is characterized by persistent redness (erythema) with a tendency to flush and blush easily. Telangiectasis is also a common symptom. Some patients report burning or itching sensations.

2. Papulopustular rosacea: This subtype is characterized by persistent redness with papules and some pus filled pustules.

3. Phymatous rosacea: This subtype is most commonly associated with rhinophyma. Symptoms also include thickening skin, irregular surface nodularities, and enlargement, primarily of the nose, chin (gnatophyma), forehead (metophyma), cheeks, eyelids (blepharophyma), and ears (otophyma). Telangiectasis may also be present.

4. Ocular rosacea: The most common symptoms of this subtype are red, dry and irritated eyes and eyelids.

Current treatments for rosacea include retinoids and antibiotics. However, these treatments are generally not suitable for long term therapy. Accordingly, the delivery of a microbe genetically modified to express one or more LEKTI protein domains encoded by one or more SPINK genes, as described herein, offers a safe and effective treatment for rosacea.

The methods of the present invention can be used in the treatment of rosacea of any subtype. Depending on the severity of the symptoms, more treatments and/or longer treatment times (time of each treatment) may be needed to produce the desired therapeutic efficacy. According to some embodiments, the methods described herein decrease the frequency and/or severity of rosacea outbreaks. Similarly, the present methods can help prevent scarring associated with rosacea.

Atopic Dermatitis

Atopic dermatitis (AD) is a recurrent, inflammatory condition often experienced by infants, children, and young adults. It begins on the cheeks and may extend to the rest of the face, neck, wrists, and hands. The most common symptoms include intense itching and very dry skin. There is currently no curative treatment for AD. Depending on the severity of the symptoms, topical steroidal antihistamines and immunomodulators or antibiotics, antivirals or antifungals are usually prescribed.

According to some embodiments, the disclosure disclosed herein provides compositions and methods for treating or preventing atopic dermatitis in a subject.

Atopic dermatitis is also known as atopic eczema. The skin of patients with this condition is especially sensitive to irritants and other allergens. The patient is thus vulnerable to skin reactions that cause red, dry, itchy skin. The itching often makes patients scratch or rub the effected tissue, and this can cause bleeding, cracking, oozing, or otherwise disrupt the skin. The open areas of skin can leave patients vulnerable to infection.

Diagnosis of atopic dermatitis can involve the use of skin biopsies and/or laboratory testing to exclude other skin disorders. A family or personal history of atopy (i.e. a genetic tendency to develop allergic diseases), ichthyosis vulgaris, and xerosis can increase the risk of developing skin disorders. Other inflammatory skin conditions and disorders that can present similarly with atopic dermatitis include, for example, contact dermatitis, seborrheic dermatitis, and psoriasis. Subjects with skin disorders can be at a greater risk of viral skin infections, for example, the potentially life threatening eczema herpeticum.

Psoriasis

Psoriasis is a chronic skin disease which is known to be difficult to treat. Psoriasis is an inflammatory skin condition caused, at least in part, by an inflammatory response in the patient. There are several major types, each with unique signs and symptoms. Between 10% and 30% of people who develop psoriasis get a related form of arthritis called “psoriatic arthritis,” which causes inflammation of the joints.

According to some embodiments, the disclosure disclosed herein provides compositions and methods for treating or preventing psoriasis in a subject.

Plaque psoriasis is the most common type of psoriasis. About 80% of people who develop psoriasis have plaque psoriasis, which appears as patches of raised, reddish skin covered by silvery-white scales. These patches, or plaques, frequently form on the elbows, knees, lower back, and scalp. However, the plaques can occur anywhere on the body. Aside from the self-consciousness and cosmetic impact of these plaques, they are also itchy and uncomfortable. At times, they may bleed and become even more noticeable. The present invention provides novel methods for treating the symptoms of psoriasis. In particular, administration of one or more LEKTI protein domains as described herein can be used to decrease the inflamed, scaly lesions associated with psoriasis. Additionally, administration of one or more LEKTI protein domains as described herein can help decrease the frequency of outbreaks.

Psoriasis is typically characterized as follows:

Plaque psoriasis (psoriasis vulgaris) is the most common form of psoriasis, accounting for 80-90% of psoriasis cases. Plaque psoriasis typically appears as raised areas of inflamed skin covered with silvery white scaly skin.

Flexural psoriasis (inverse psoriasis) appears as smooth inflamed patches of skin. It typically occurs in skin folds, such as around the genitals, armpits, or under the breasts.

Guttate psoriasis is characterized by numerous small oval (teardrop-shaped) spots. These numerous spots of psoriasis appear over large areas of the body, such as the trunk, limbs, and scalp. This type of psoriasis is associated with streptococcal throat infection, further supporting the link between psoriasis and the immune response.

Pustular psoriasis appears as raised bumps that are filled with non-infectious pus (pustules). The skin under and surrounding pustules is red and tender. Pustular psoriasis can be localized, generally to the hands and feet, or it can occur as patches occurring randomly on any part of the body.

Nail psoriasis produces changes in the appearance of finger and toe nails. Symptoms include discoloration, pitting, thickening of the skin under the nail, loosening of the nails, and crumbling of the nails.

Psoriatic arthritis involves joint and connective tissue inflammation, generally the joints of the fingers and toes. About 10-15% of people who have psoriasis also have psoriatic arthritis.

Erythrodermic psoriasis involves the widespread inflammation and exfoliation of the skin over most of the body surface. It may be accompanied by severe itching, swelling and pain. It is often the result of an exacerbation of unstable plaque psoriasis, particularly following withdrawal of systemic treatment. This form of psoriasis can be fatal, as the extreme inflammation and exfoliation disrupts thermo-regulation and the barrier function of the skin.

The methods of the present disclosure can be used in the treatment or prevention of psoriasis of any subtype. Depending on the severity of the symptoms, more treatments and/or longer treatment times (time of each treatment) may be needed to produce the desired therapeutic efficacy. Although dramatic improvement in patient appearance may take multiple treatments, even a single treatment delivers therapeutically effective doses that penetrate the skin and begin to act on patient tissue. Overtime, the therapeutic efficacy of the individual treatments are additive or even synergistic, thus resulting in a decrease or elimination of symptoms and/or a lessening in the frequency of symptoms. In the case of psoriasis, this includes not only the skin-related symptoms, but also the arthritis symptoms experienced by some sufferers.

Exemplary symptoms of psoriasis include, but are not limited to, expression of one or more markers of the inflammatory response, bacterial count, swelling, redness, itchiness, pain, number of lesions, frequency of outbreaks of lesions, severity of outbreaks of lesions, skin dryness, skin flaking, and skin discoloration.

In certain embodiments, the treated subject tissue is tissue of one or more of the head, face (e.g., cheeks, chin, forehead, nose, etc.), arms, hands, legs, or torso. In certain embodiments, the treated subject tissue is tissue of the face. In certain embodiments, the treated subject tissue is tissue of the arms or hands. In certain embodiments, the treated subject tissue is tissue of the legs. In certain embodiments, the treated subject tissue is tissue of the torso.

In certain embodiments, the treated subject tissue comprises cells found in the gut, sinuses, vaginal, ocular, ears, or nose.

In certain embodiments, the treated subject tissue comprises cells found in a wound.

Pruritus

The transient receptor potential (TRP) channels comprise 28 members in mammals that can be divided into six subfamilies based on amino acid sequence homology, including TRPA, TRPC, TRPM, TRPML, TRPP, and TRPV (Montell et al. Cell. 2002 Mar. 8; 108(5):595-8). TRP channels are molecular sensors of mechanical, chemical, and thermal environmental cues and are crucially involved in both acute and chronic itch (Dong. Neuron. 2018 May 2; 98(3):482-494). Six TRP channels are now firmly associated with itch generation and transduction. They are implicated in many sensory functions including taste, smell, thermoception, touch, osmolarity, and pain (Venkatachalam K, Montell C Annu Rev Biochem. 2007; 760:387-417; Zheng J Compr Physiol. 2013 January; 3(1):221-4; Wu et al. Pharmacol Rev. 2010 September; 62(3):381-404). In the past two decades, numerous studies have demonstrated that TRP channels are critically involved in itch sensation under both physiological and pathological conditions (Steinhoff M, Biro T J Invest Dermatol. 2009 March; 129(3):531-5; Moore et al. Neurosci Bull. 2018 February; 34(1):120-142).

TRPV1 belongs to a subfamily of temperature-sensitive TRP channels, also called “ThermoTRPs” (Kim et al. PLoS One. 2013; 8(3):e59593). TRPV1 is activated by noxious temperatures (>43° C.) (Rosenbaum T., Simon S. A. In: Liedtke W. B., Heller S., editors. CRC Press; Boca Raton, Fla., USA: 200). In addition, TRPV1 is activated by capsaicin, low pH, and numerous molecules associated with inflammation and tissue damage such as bradykinin, prokineticin, prostaglandins, anandamide, and retinoids (Luo et al. Cell Mol Life Sci. 2015 September; 72(17):3201-23; Carnevale V, Rohacs T. Pharmaceuticals (Basel). 2016 Aug. 23; 9(3); Yin et al. J Clin Invest. 2013 September; 123(9):3941-51). As the best-characterized itch mediator, histamine is released from mast cells and binds H1/H4 receptors on skin nerve terminals to elicit itch (Shim, W. S., and Oh, U. 2008. Mol. Pain 4:29), via activation the PLCbeta3 and transient receptor potential subtype VI (TRPV1)(Imamachi et al. Proc. Natl. Acad. Sci. U. S. A 106: 11330-11335).

The sensation of itch is one of the most common skin problems experienced by humans and animals. Itch can be defined as a sensation which provokes the desire to scratch the site from which the sensation originates. All skin contains sensory nerves which transmit itch in response to chemical irritation, environmental exposure or disease processes. Although the precise population of itch producing nerves have not been identified, the thinnest, unmyelinated nerve population, termed type C nociceptive neurons are thought to be the most important in producing the sensation. Itch: Mechanisms and Management of Pruritus. Jeffrey D. Bernhard. McGraw-Hill, Inc. (San Francisco, 1994), pp. 1-22. The itch-producing nerves of the skin can be considered to be a “final common pathway” for the many irritating conditions which are ultimately sensed as itch including chemical exposure, environmental exposure (such as that which produces dry, itchy skin) and disease processes such as atopic dermatitis. Many chemical substances are able to produce itch when topically applied to the skin. No matter what the ultimate cause of itch, the sensation experienced is the same and provokes the desire to scratch.

Conventional treatments of pruritus include prescription and over-the-counter medications, including topical anti-inflammatory agents, anti-histamines, and emollients, which are aimed at managing the itchy sensation. However, without eradication of the underlying disease, treatment of pruritus often is ineffective and may be frustrating for patients and prescribing physicians. Thus, the treatment of pruritus continues to be a diagnostic and therapeutic challenge, such that there is a need for improved compositions and methods for treating pruritus.

The present disclosure is based in part on the finding that administering one or more LEKTI protein domains can be used to decrease the pruritic response.

According to some embodiments, the pruritic response occurs in cells found in the gut, sinuses, vaginal, ocular, ears, or nose. According to some embodiments, In certain embodiments, the pruritic response occurs in cells found in a wound.

As such, the methods of the present disclosure can be used to treat or prevent one or more symptoms of pruritus in a subject. According to some embodiments, the symptoms of the pruritic response include itching, stinging, burning, tingling, tightness, erythema (redness), or edema (swelling). Accordingly, the methods of the present disclosure can be used to treat or prevent pruritus in a subject.

the methods described herein can provide any amount of any level of treatment or prevention of pruritus in a mammal. Furthermore, the treatment or prevention provided by the disclosed methods can include treatment or prevention of one or more conditions or symptoms of the pruritus, e.g., chronic pruritus, being treated or prevented. Also, for purposes herein, “prevention” can encompass delaying the onset of the pruritus, or a symptom or condition thereof. With respect to the methods described herein, the pruritus can be any pruritus, including any of the types of pruritus caused by or associated with any of the conditions or treatments discussed herein.

There are six general categories of pruritus: dermatologic, systemic, neurogenic, psychogenic, mixed, and other. Among the most widely known dermatological mechanisms of itch is through the release of histamine from mast cells and basophils. This can occur immunologically when allergens bind to immunoglobin E found on the surface of mast cells and basophils which leads to the release of histamine prestored in granules. Histamine can also be released from mast cells and basophils through non-immunologically mediated mechanisms such as cold, stress, and certain chemicals described below. Histamine also increases surface wound blood flow, which would explain the raised red surface usually present on the chronically itching wound. Histamine directly stimulates histamine type 1 (H1)-receptors on itch-specific neurons and can induce the classic wheal-and-flare response. While the wheal is a response to H1-receptor stimulation, the flare is the result of the secondary release of vasoactive substances from collateral axons. The wheal-and-flare response is specific for histamine-mediated itch. Histamine is the mediator for itch in several conditions, including: (i) most forms of urticaria; (ii) insect bite reactions; (iii) cutaneous mastocytosis; and (iv) drug rashes, e.g., antibiotics. The involvement of histamine is confirmed by the antipruritic effect of low-sedative H1-antihistamines in these conditions. The main source of histamine in the skin is the dermal mast cell from which it is released by mast cell degranulation. See, e.g., Paus et al., “Frontiers in pruritus research: scratching the brain for more effective itch therapy,” J. Clin. Invest., 116(4):1174-1185 (May 2006).

Several other compounds can induce the itch response. Acetylcholine stimulates histamine-sensitive and histamine-insensitive neurons. Serotonin (5-hydroxytryptamine, 5HT) can cause itch by both peripheral and central mechanisms. Peripherally, it acts indirectly through the release of histamine from dermal mast cells. Prostaglandins are not themselves pruritogenic, but potentiate itching caused by histamine and probably other mediators.

Cytokines are also major itch factors. For example several hours after interleukin-2 (IL2) is injected intradermally in both atopic and non-atopic subjects, itch and erythema occur and last 2-3 days. When given intravenously with cytotoxic drugs in the treatment of malignant melanoma, IL-2 causes intense itch.

Neuropeptides such as Substance P potentiate itch. Substance P is a short-chain polypeptide that functions as a neurotransmitter and as a neuromodulator. It belongs to the tachykinin neuropeptide family. It is unclear if substance P induces histamine release from mast cells to induce itch or if it induces itch by itself. For example substance P can directly induce human skin mast cells to release histamine (see, e.g., Oskeritzian et al., “Surface CD88 functionally distinguishes the MC from the MC type of human lung mast cell,” J. Allergy and Clin. Immunol., 115(6):1162-1168 (2005)) and elicits an itch response in both humans and mice (see, e.g., Hagermark et al., “Flare and Itch Induced by Substance P in Human Skin,” J. of Investigative Dermatology, 71:233-235 (1978); Barnes et al., “Plasma histamine levels following atracurium,” Anaesthesia, 41(8):821-824 (1986); Kuraishi et al., “Scratching behavior induced by pruritogenic but not algesiogenic agents in mice,” European Journal of Pharmacology, 275(3):229-233 (1995); Andoh et al., “Substance P Induction of Itch-Associated Response Mediated by Cutaneous NK1 Tachykinin Receptors in Mice,” J. of Pharmacology and Experimental Therapeutics, 286(3):1140-1145 (1998)). However, in mice intradermal injection of substance P itch-associated response is not inhibited by antihistamines. See, e.g., Andoh et al., “Involvement of Leukotriene B4 in Substance P-Induced Itch-Associated Response in Mice,” J. of Investigative Dermatology, 117:1621-1626 (2001)).

According to some embodiments, the pruritus is acute pruritus. According to some embodiments, the pruritus is chronic pruritus. According to some embodiments, the pruritus is mild pruritus. Mild and acute pruritus, like pain, can serve a protective function, but chronic pruritus can have a significant negative impact on the quality of life of a subject. According to some embodiments, the pruritus may be widespread or localized on a subject's body.

According to some embodiments, the pruritus may be caused by or associated with any condition or any treatment of a condition. According to some embodiments, the pruritus may be caused by or associated with a skin condition. According to some embodiments, the pruritus may be caused by or associated with a systemic condition or treatment of a systemic condition.

Pruritus is generally a common symptom of skin diseases. Systemic diseases and other conditions may also cause pruritus. For example, acute or chronic pruritus can be a common and disabling problem in many burn victims with healed burn wounds. In addition pruritus can be caused and/or aggravated by, but not limited to, the following: dry skin, allergic reactions, allergies, insect bites and stings, insect allergies, tick bites, flea bite, worm allergies (e.g., threadworm, etc.), irritating chemicals, parasites (e.g., pinworms, scabies, lice, etc.), pregnancy, rashes, reactions to medicines, rash, itchy rash, skin inflammation, blisters, hives, eczema, contact dermatitis, poison oak, poison ivy, shingles, fungal and/or bacterial infection, lichen simplex, pityriasis, rosea, lichen sclerosis et atrophicus, nodular prurigo, vulval itch, chicken pox, measles, itch, anal itch, genital itch, reaction to medication, food allergy, jaundice, leukemia, polycythemia, kidney disease, hypothyroidism, hyperthyroidism, senile pruritus, fibers (e.g., fiber glass), psoriasis, eczema (atopic dermatitis), dermatitis (e.g., dermatitis herpetiformis, solar dermatitis, etc.), sun burn, jaundice, liver diseases, uremia, polycythemia vera, lymphoma, Hodgkin's lymphoma, leukemia, jock itch, feminine itch, and psychogenic itch.

The following list of exemplary conditions can cause symptoms of pruritus: acute kidney failure, allergies, anaphylaxis, arthritis, athlete's foot, autoimmune hepatitis, blepharitis, candidiasis, cercarial dermatitis, chickenpox, chilblain, cholangitis, cholecystitis, chronic kidney failure, ciguatera poisoning, cirrhosis of the liver, cutaneous mastocytosis, decompression sickness, dermatitis, dermatitis herpetiformis, diabetes, drug allergies, dry skin, eczema, food allergies, heat rash, Hodgkin's disease, hyper-IgE syndrome, hyperthyroidism, hypothyroidism, jaundice, lichen sclerosis, lymphoma, mastocytosis, molluscum contagiosum, mycosis fungoides, non-Hodgkin's lymphoma, osteoarthritis, pancreatic cancer, pediculosis, pityriasis rosea, polycythemia, porphyria, primary biliary cirrhosis, primary sclerosing cholangitis, psoriasis, rabies, scabies, schistosomiasis, Sjogren's syndrome, slap-cheek syndrome, type 2 diabetes, Wiskott-Aldrich Syndrome, and yaws.

Exemplary causes of localized pruritus include eczema, contact dermatitis, poison oak, poison ivy, insect bite, insect sting, parasites, scabies, lice, tick bite, shingles. Examples of diseases that cause an itchy rash include: hives, blisters, eczema, lice, scabies, insect bite, insect sting, fungal infection, lichen simplex, pityriasis, rosea, lichen sclerosis et atrophicus, nodular prurigo, vulval itch, chicken pox, measles. Adverse reactions to medications include certain medications, see also causes of itchy rash, itch, anal itch, vulval itch, genital itch. Some possible causes of itching all over include allergic reaction (type of an adverse reaction), reaction to medication, food allergy, insect allergy, jaundice, leukemia, polycythemia, kidney disease, dry skin, hypothyroidism. Some diseases that cause itch without a rash include lice, scabies, insect bite, threadworm, flea bite, senile pruritus, fiber glass fibers, pregnancy, dermatitis herpetiformis, jaundice, liver diseases, uremia, polycythemia vera, lymphoma, Hodgkin's lymphoma, leukemia, psychogenic itch, and certain medications. The methods of treating pruritus in a subject in need thereof are disclosed herein. The methods described herein disclose treating pruritus that is (i) immunologically-mediated, (ii) histamine-induced, (iii) non immunologically-mediated, (iv) parasite-induced, (v) fungus and bacteria induced, (vi) induced by insect bites, (vii) induced by plant derived pruritogens, and/or (viii) induced by poison ivy.

According to some embodiments, pruritus can be caused and/or aggravated by dry skin, allergic reactions, allergies, insect bites and stings, insect allergies, tick bites, flea bite, worm allergies (e.g., threadworm, etc.), irritating chemicals, environmental irritants, parasites (e.g., pinworms, scabies, lice, etc.), pregnancy, rashes, reactions to medicines, rash, itchy rash, skin inflammation, blisters, hives, eczema, contact dermatitis, poison oak, poison ivy, shingles, fungal and/or bacterial infection, lichen simplex, pityriasis, rosea, lichen sclerosis et atrophicus, nodular prurigo, vulval itch, chicken pox, measles, itch, anal itch, genital itch, reaction to medication, food allergy, jaundice, leukemia, polycythemia, kidney disease, hypothyroidism, hyperthyroidism, senile pruritus, fibers (e.g., fiber glass), psoriasis, eczema (atopic dermatitis), dermatitis (e.g., dermatitis herpetiformis, solar dermatitis, etc.), sun burn, jaundice, liver diseases, uremia, polycythemia vera, lymphoma, Hodgkin's lymphoma, leukemia, jock itch, feminine itch, and/or psychogenic itch. Other causes of pruritus include: chiggers, the larval form of which secretes substance that creates a red papule that itches intensely; secondary hyperparathyroidism associated with chronic renal failure; cutaneous larva migrans, caused by burrowing larvae of animal hookworms; dermal myiasis, caused by maggots of the horse botfly, which can afflict horseback riders; onchocerciasis (“river blindness”) caused by filarial nematodes; pediculosis, caused by lice infestations; enterobiasis (pinworm) infestations, which afflict millions of Americans, particularly school children; schistosome dermatitis (swimmer's itch); psoriasis; poison ivy; and asteatotic eczema (“winter itch”).

The present disclosure provides a method of treating pruritus in a subject,

comprising administering a microbe genetically modified to express one or more LEKTI protein domains encoded by one or more SPINK genes to a subject to provide a therapeutic effect. According to some embodiments, treating pruritus is intended to encompass treating itch.

The present disclosure provides a method of preventing pruritus in a subject, comprising administering a microbe genetically modified to express one or more LEKTI protein domains encoded by one or more SPINK genes to a subject to provide a therapeutic effect. According to some embodiments, preventing pruritus is intended to encompass preventing or preventing the progression of itch).

The present disclosure provides a method of treating pruritus in a subject, comprising administering a microbe genetically modified to express one or more LEKTI protein domains encoded by one or more SPINK genes to the skin of a subject in need thereof. According to some embodiments, the microbe genetically modified to express one or more LEKTI protein domains penetrates the skin to provide a therapeutic effect. According to some embodiments, the microbe genetically modified to express one or more LEKTI protein domains encoded by one or more SPINK genes is administered on the skin and the LEKTI protein domains penetrate the skin to provide a therapeutic effect. According to some embodiments, treating pruritus is intended to encompass treating itch.

The present disclosure provides a method of preventing pruritus in a subject, comprising administering a microbe genetically modified to express one or more LEKTI protein domains encoded by one or more SPINK genes to the skin of a subject in need thereof. According to some embodiments, the microbe genetically modified to express one or more LEKTI protein domains penetrates the skin to provide a therapeutic effect. According to some embodiments, the microbe genetically modified to express one or more LEKTI protein domains encoded by one or more SPINK genes is administered on the skin and the LEKTI protein domains penetrate the skin to provide a therapeutic effect. According to some embodiments, preventing pruritus is intended to encompass preventing or preventing the progression of itch.

The present disclosure provides a method for treating a disease or disorder having itch as a symptom or sensation associated with the disease or disorder in a subject, comprising administering a microbe genetically modified to express one or more LEKTI protein domains to provide a therapeutic effect to decrease itch.

The present disclosure provides a method for treating a disease or disorder having itch as a symptom or sensation associated with the disease or disorder in a subject, comprising administering a microbe genetically modified to express one or more LEKTI protein domains encoded by one or more SPINK genes to the skin of a subject in need thereof. According to some embodiments, the microbe genetically modified to express one or more LEKTI protein domains penetrates the skin to provide a therapeutic effect to decrease itch. According to some embodiments, the microbe genetically modified to express one or more LEKTI protein domains encoded by one or more SPINK genes is administered on the skin and the LEKTI protein domains penetrate the skin to provide a therapeutic effect.

The present disclosure provides a method of preventing or treating one or more symptom or sensation arising from an irritation, hives, pain, inflammation, asthma, allergy, or allergic rhinitis in a subject, comprising administering a microbe genetically modified to express one or more LEKTI protein domains encoded by one or more SPINK genes to provide a therapeutic effect. According to some embodiments, the symptom or sensation is itch. According to some embodiments, the therapeutic effect is decreasing itch, preventing itch, or preventing the progression of itch.

The present disclosure provides a method of preventing or treating one or more symptom or sensation arising from an irritation, hives, pain, inflammation, asthma, allergy, or allergic rhinitis in a subject, comprising administering a microbe genetically modified to express one or more LEKTI protein domains encoded by one or more SPINK genes domains to the skin of a subject in need thereof. According to some embodiments, the microbe genetically modified to express one or more LEKTI protein domains penetrates the skin to provide a therapeutic effect. According to some embodiments, the microbe genetically modified to express LEKTI protein domains encoded by one or more SPINK genes is administered on the skin and the LEKTI protein domains penetrate the skin to provide a therapeutic effect. According to some embodiments, the symptom or sensation is itch. According to some embodiments, the therapeutic effect is decreasing itch, preventing itch, or preventing the progression of itch.

According to some embodiments, the therapeutic effect includes increasing healing, and increasing proliferation of healthy skin tissue. Over the course of therapy, therapeutic efficacy can be assessed by evaluating decrease in the presence or severity of the itch.

According to some embodiments, the method for treating or preventing pruritus comprises multiple treatments (e.g., 1, 2, 3, 4, 5, 6, 7, 8, 9, 10, 15, 20, 25, 20, 35, 40, 45, 50, 55, 60, 65, 70, 75, 80, 85, 90, 95, 100 or more treatments).

The appropriate number of treatments, and the duration of each treatment, can be determined by a health care provider based on, for example, the severity of the disorder, and the overall health of the subject.

According to some embodiments, the method is part of a therapeutic regimen combining one or more additional treatment modalities as part of a therapeutic regimen for treating pruritus in a subject. When used in this way, administration of a microbe genetically modified to express one or more LEKTI protein domains encoded by one or more SPINK genes can act additively or synergistically with other treatments. Exemplary therapies include, but are not limited to, antibiotics, hydrocortisone creams, benzoil peroxide, retinoids and other vitamin A based agents, steroids or other immunosuppressive agents (methotrexate, cyclosporin), and the like.

In certain embodiments, the treated subject tissue is tissue of one or more of the head, face (e.g., cheeks, chin, forehead, nose, etc.), arms, hands, legs, or torso. In certain embodiments, the treated subject tissue is tissue of the face. In certain embodiments, the treated subject tissue is tissue of the arms or hands. In certain embodiments, the treated subject tissue is tissue of the legs. In certain embodiments, the treated subject tissue is tissue of the torso.

According to some embodiments, itch (e.g. decrease or increase of itch) can be measured by subjective measures. According to some embodiments, itch is measured using the Visual Analog Scale (VAS) (Reich et al., Acta Derm Venereol 2016; 96: 978-980). The VAS is a scale consisting of a 10 cm long line and a single question. Along with the NRS, it is most commonly used in clinical trials for measuring itch intensity and features high reliability and concurrent validity. The left end point represents “no itch” and the right end point the “worst imaginable itch”. It can be interpreted as follows: VAS 0=No pruritus; VAS<3=Mild pruritus; VAS≥3-<7=Moderate pruritus; VAS≥7-<9=Severe pruritus; VAS≥9=Very severe pruritus.

According to some embodiments, itch can be measured using the 5-D itch questionnaire. The 5-D itch questionnaire was developed to be a measure of itch that is brief (one page), easy to complete, easy to score (either manually at the bedside or electronically as part of a large clinical trial), sensitive to the multidimensional nature of pruritus and its effect on quality of life, applicable to multiple diseases, and capable of detecting change over time (Elman et al. Br J Dermatol. 2010 March; 162(3): 587-593)

According to some embodiments, itch can be measured using the 12-Item Pruritus Severity Score (12-PSS). The PSS assess pruritus intensity (two questions), pruritus extent (one question) and duration (one question), influence of pruritus on concentration and patient psyche (four questions), and scratching as a response to pruritus stimuli (four questions) (Reich et al. Biomed Res Int. 2017; 2017: 3896423)

According to other embodiments, another approach is to measure scratch which is an objective correlate of itch using a vibration transducer or movement-sensitive meters.

According to some embodiments, any one or combination of the above measures can be used to measure itch (e.g. decrease or increase of itch) in a subject.

### Pain

Pain is one of the most frequent symptoms for which patients seek medical help. Pain can be classified as acute or chronic. Acute pain is generally associated with excessive noxious stimulus resulting in a severe distressful sensation. Chronic pain is associated with physiological changes as a result of tissue or nerve injury leading to hyperalgesia, an increased amount of pain associated with a mild noxious stimulus, or allodynia, a pain induced by a non-noxious stimulus. Neuropathic pain is a neurological disease caused by the damage to the somatosensory pathway that produces severe chronic pain. While pain associated with tissue injury is self-limiting, neuropathic pain is long lasting, and may develop days or month after the injury. This type of chronic pain is observed in diseases affecting the central nervous system such as stroke and multiple sclerosis or with conditions related to peripheral nerve damage such as diabetic neuropathy.

The therapeutic objective of most pain therapy is to alleviate the symptoms of pain regardless of the cause. Current pain-control therapies include the use of opioid narcotic analgesics such as morphine and fentanyl, nonsteroidal anti-inflammatory drugs (NSAIDS) such as aspirin, ibuprofen and cyclooxygenase inhibitors, or ion channel blockers such as lidocaine and novocaine. These therapies all have limitations, however. Opioids can cause tolerance, dependence, constipation, respiratory depression and sedation. NSAIDS have gastrointestinal side effects, can increase bleeding time, and are not effective in the treatment of severe pain. In the case of non-selective sodium channel blockers, central nervous system (CNS) side effects, cardiovascular side effects and corneal damage have been reported after use.

Despite a wide range of available medical treatments, pain continues to afflict millions of individuals in the United States alone and remains a profound burden to patients, health care, and business.

The present disclosure provides methods that can be used to treat, prevent or reduce pain in a subject.

The term “pain” as used herein is intended, generally, to represent all categories of physical pain. This includes traumatic pain resulting from injury, surgery or inflammation. It also includes pain associated with diseases such as cancer, AIDS, arthritis, and herpes. Pain associated with neuropathy such as diabetic neuropathy, causalgia, brachial plexus avulsion, occipital neuralgia, fibromyalgia, vulvodynia, prostadynia, pelvic pain, gout, and other forms of neuralgia, such as neuropathic and idiopathic pain syndromes are also included. Specific organ- or site-localized pain, such as headache, ocular and corneal pain, bone pain, urogenital pain, heart pain, skin/burn pain, lung pain, visceral (kidney, gall bladder, etc.) pain, joint pain, dental pain and muscle pain are further included in this invention. The general term “pain” also covers pain symptoms of varying severity, i.e. mild, moderate and severe pain, as well as those of acute and chronic pain. According to some embodiments, the methods of treating, reducing, or preventing pain described herein include pain associated with traumatic pain, neuropathic pain, inflammatory pain, acute pain, chronic pain, organ or tissue pain, and pain associated with diseases, such as cancer.

According to some embodiments, the pain is acute pain.

According to some embodiments, the pain is chronic pain.

According to some embodiments, the pain is nociceptive pain. According to some embodiments, the pain is neuropathic pain. Nociceptive pain differs from neuropathic pain in that an external stimulus causes a normal sensory response to an insult or illness in the case of traumatic pain, whereas neuropathic pain results from injury to a portion of the nervous system and is typically not responsive to narcotic analgesics. Neuropathic pain often involves neural hypersensitivity and can persist without any overt external stimulus. (Goodman & Gilman's “The Pharmacologic Basis of Therapeutics”, 1996, p. 529, McGraw-Hill).

The method of the present disclosure alleviates the symptoms of pain regardless of the cause of the pain. Pain treatable by the present method includes traumatic pain, neuropathic pain, organ and tissue pain, and pain associated with diseases. Traumatic pain includes pain resulting from injury, post-surgical pain and inflammatory pain. Neuropathic pain includes neuropathic and idiopathic pain syndromes, and pain associated with neuropathy such as diabetic neuropathy, causalgia, brachial plexus avulsion, occipital neuralgia, fibromyalgia, gout, and other forms of neuralgia. Organ or tissue pain includes headache, ocular pain, corneal pain, bone pain, heart pain, skin/bum pain, lung pain, visceral pain (kidney, gall bladder, etc.), joint pain, dental pain, muscle pain, pelvic pain, and urogenital pain (e.g. vulvodynia and prostadynia). Pain associated with diseases includes pain associated with cancer, AIDS, arthritis, herpes and migraine.

The present disclosure provides methods of treating pain in a subject in need thereof, comprising administering one or more LEKTI protein domains to a subject to treat the symptoms of pain. According to some embodiments, the present disclosure provides a method of treating pain in a subject in need thereof, comprising administering a microbe genetically modified to express one or more LEKTI protein domains encoded by one or more SPINK genes to a subject to treat the symptoms of pain. According to some embodiments, treating pain comprises reducing the symptoms of pain.

The present disclosure provides a method of preventing pain in a subject in need thereof, comprising administering one or more LEKTI protein domains to a subject to prevent the symptoms of pain. According to some embodiments, the present disclosure provides a method of preventing pain in a subject in need thereof, comprising administering a microbe genetically modified to express one or more LEKTI protein domains encoded by one or more SPINK genes to a subject to prevent the symptoms of pain.

According to some embodiments, treating pain comprises reducing the symptoms of pain.

According to some embodiments, the methods for treating, preventing or reducing pain comprise multiple treatments (e.g., 1, 2, 3, 4, 5, 6, 7, 8, 9, 10, 15, 20, 25, 20, 35, 40, 45, 50, 55, 60, 65, 70, 75, 80, 85, 90, 95, 100 or more treatments).

The appropriate number of treatments, and the duration of each treatment, can be determined by a health care provider based on, for example, the severity of the pain and the overall health of the subject.

According to some embodiments, the method is part of a therapeutic regimen combining one or more additional treatment modalities as part of a therapeutic regimen for treating pain. When used in this way, administration of one or more LEKTI protein domains can act additively or synergistically with other treatments. Exemplary therapies include, but are not limited to, low dose colchicine, aspirin, steroids such as prednisolone, methotrexate, low dose cyclosporine A, TNF inhibitors, other inflammatory inhibitors such as inhibitors of caspase-1, p38, IKK1/2, CTLA-4lg, anti-IL-6 or anti-IL6Ra, etc., and/or co-therapies such as uric acid synthesis inhibitors to inhibit the accumulation of uric acid in the body, for example, allopurinol, uric acid excretion promoters to accelerate the rapid excretion of uric acid accumulated in the body, for example, probenecid, sulfinpyrazone and/or benzbromarone are examples of uric acid excretion promoters; corticosteroids; and other non-steroidal anti-inflammatory drugs (NSAIDs).

In order to measure the efficacy of any of the methods, compositions, or kits of the invention, a measurement index may be used. Indices that are useful in the methods, compositions, and kits of the invention for the measurement of pain associated with musculoskeletal, immunoinflammatory and neuropathic disorders include a visual analog scale (VAS), a Likert scale, categorical pain scales, descriptors, the Lequesne index, the WOMAC index, and the AUSCAN index, each of which is well known in the art. Such indices may be used to measure pain, itch, function, stiffness, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain or itch by choosing the spot on the line that best corresponds to the sensation of pain or itch, where one end of the line corresponds to “no pain” (score of 0 cm) or “no itch” and the other end of the line corresponds to “unbearable pain” or “unbearable itch” (score of 10 cm). This procedure provides a simple and rapid approach to obtaining quantitative information about how the patient is experiencing pain or itch. VAS scales and their use are described, e.g., in U.S. Pat. Nos. 6,709,406 and 6,432,937, incorporated by reference in their entireties herein.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS); the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

In certain embodiments, the treated subject tissue is tissue of one or more of the head, face (e.g., cheeks, chin, forehead, nose, etc.), arms, hands, legs, or torso. In certain embodiments, the treated subject tissue is tissue of the face. In certain embodiments, the treated subject tissue is tissue of the arms or hands. In certain embodiments, the treated subject tissue is tissue of the legs. In certain embodiments, the treated subject tissue is tissue of the torso.

### 6. Animal Models

The use of one or more LEKTI protein domains (in the presence or absence of additional therapeutic modalities) to treat or prevent one or more symptoms of a skin disease or disorder can be tested in one or more animal models. Exemplary animal models are described briefly herein. However, numerous animal models exist and any model available in the art can be readily used to evaluate a particular treatment regimen (e.g., to evaluate number of treatments, duration of treatment, combination with one or more current treatment modalities).

Numerous models for dermatitis exist and can be used. The NC/Tnd murine atopic dermatitis model, the keratin 14 IL-4 transgenic mouse model and the WBN/Kob-Ht rat model are commonly used. See, for example, Chen et al. (2005) Clin Exp Immunolo 142: 21-30 and Asakawa et al. (2005) Exp Animals 54: 461-465. Additional animal models are summarized in Nishimuta and Ito (2003) Archives of Dermatol Res 294: 544-551, all of which are incorporated by reference in their entireties herein.

Numerous animal models of psoriasis exist. 17 rodent models are summarized by Schon. See, Schon (1999) Society of Investigative Dermatology 112: 405-410, incorporated by reference in its entirety herein. These animal models include spontaneous mutant models, transgenic animals, knock-out animals, and xenotransplantation models.

Additional animal models (including mouse, rat, porcine, and canine models) of various inflammatory skin disorders, as well as more detailed descriptions of many of the foregoing models, are provided in “Animal Models of Human Inflammatory Skin Diseases”, Lawrence S. Chan; published by Informa Health Care, Dec. 29, 2003, incorporated by reference in its entirety herein.

Therapeutic regimens comprising administration of one or more LEKTI protein domains (including one or more treatments with a microbe genetically modified to express one or more LEKTI protein domains encoded by one or more SPINK genes (alone or in combination with one or more additional treatment modalities)) can be tested in one or more animal models. Exemplary models are described herein, although numerous additional animal models are well known in the art and can be similarly used. Treatment with one or more LEKTI protein domains is compared to, for example, no treatment controls or control treatment with one or more current therapies. Additionally or alternatively, such models can be used to assess, for example, the effectiveness of a therapeutic regimen in which the frequency of treatments and/or the duration of each treatment are varied.

Additionally, therapeutic regimens comprising administration of one or more LEKTI protein domains (including one or more treatments with a microbe genetically modified to express one or more LEKTI protein domains encoded by one or more SPINK genes (alone or in combination with one or more additional treatment modalities)) can be tested in in vitro models (e.g., cell-based models, organ culture models). Further, such therapeutic regimens can be tested in vivo in human patients.

The use of one or more LEKTI protein domains (in the presence or absence of additional therapeutic modalities) to treat or prevent pain can be tested in one or more animal models. In human patients, a distinction is made between stimulus-evoked pain and stimulus-independent or spontaneous pain. Stimulus-evoked pain is described as either hyperalgesia or allodynia, and is further subdivided on the basis of the evoked stimulus modality (e.g., mechanical, heat, cold, chemical; Woolf and Mannion, Lancet. 1999 Jun. 5; 353(9168):1959-64). Hyperalgesia is defined as an increased or exaggerated pain response to a normally noxious stimulus, while allodynia is defined as a painful response to a normally non-noxious or innocuous stimulus. In cases of sensory loss, hypoalgesia may be present, which is defined as decreased sensitivity to a nociceptive stimulus. Stimulus-evoked pain can be evaluated in humans using quantitative sensory testing. While not in routine clinical use, quantitative sensory testing has the potential to improve patient outcomes by classifying pain based on the mechanism and choosing treatments that target that mechanism (Baron et al., Lancet Neurol. 2010 August; 9(8):807-19; Cruz-Almeida and Fillingim, Pain Med. 2014 January; 15(1):61-72).

Exemplary animal models are described briefly herein. However, numerous animal models exist and any model available in the art can be readily used to evaluate a particular treatment regimen (e.g., to evaluate number of treatments, duration of treatment, combination with one or more current treatment modalities).

Pain Induced by Mechanical Stimuli

Mechanical hyperalgesia and allodynia can be further subdivided into dynamic (triggered by brushing), punctate (triggered by touch) and static (triggered by pressure). Dynamic mechanical allodynia and hyperalgesia can be assessed by brushing the skin with a cotton bud, paintbrush or cotton ball, and in the case of allodynia, can be evoked by the brushing of clothing, bed sheets or towels against the skin (Jensen and Finnerup, 2014). Punctate mechanical allodynia and hyperalgesia can be evoked with a pinprick or monofilament, and in practice can be assessed by the application of von Frey filaments of varying forces (0.08-2940 mN). Static hyperalgesia can be superficial or deep and is assessed by the application of pressure to the skin or underlying tissue by a finger or using a pressure algometer (Jensen and Finnerup, Lancet Neurol. 2014 September; 13(9):924-35).

The presence and extent of aversive behaviors in responses to mechanical stimuli is typically determined using manual or electronic Von Frey or the Randall Selitto test (Arch Int Pharmacodyn Ther. 1957 Sep. 1; 111(4):409-19).

Pain Induced by Heat Stimuli

The exposure of peripheral sensory nerve endings to elevated temperatures can evoke sensations of warm, hot, or pain. Heat thresholds in humans can be determined by applying a metal probe to the skin that increases in temperature (starting at 32° C.) until a warm-sensation threshold and heat-pain threshold is reached. Typically, the sensation of warm is elicited at temperatures of 34-37° C., while the sensation of pain is elicited at temperatures of 42-48° C. (Pertovaara et al., Exp Brain Res. 1996; 107(3):497-503; Rolke et al., Pain. 2006 August; 123(3):231-43).

The tail flick test, first described in 1941, involves application of a heat stimulus to the tail of mice and rats, and the time taken for the tail to “flick” or twitch is recorded (D'Amour and Smith, J. Pharmacol. Exp. Ther. 72, 74-79). The hot plate test, first described in 1944, can be used to determine heat thresholds in mice and rats (Woolfe and Macdonald, J. Pharmacol. Exp. Ther. 80, 300-307). The dynamic hot plate test, first described in 1984, uses an increasing temperature ramp rather than a constant temperature. In this test, the unrestrained mouse or rat is placed on a metal surface starting at a non-noxious temperature (<42° C.), and the temperature is increased at a constant rate until a nocifensive behavior is observed. The temperature at which this occurs is designated as the response temperature (Ogren and Berge, Neuropharmacology. 1984 August; 23(8):915-24; Tjolsen et al., J Pharmacol Methods. 1991 May; 25(3):241-50). The Hargreaves test, first described in 1988, is a method used to quantify heat thresholds in the hind paws of mice and rats upon application of a radiant or infrared heat stimulus (Hargreaves et al., Pain. 1988 January; 32(1):77-88). The thermal probe test (MouseMet Thermal, Topcat Metrology) is a method recently described to quantify heat thresholds in mice (Deuis and Vetter, Temperature (Austin). 2016 April-June; 3(2):199-207).

Pain Induced by Cold Stimuli

Cold thresholds in humans can be determined in a similar manner to heat thresholds, where a metal probe is applied to skin that decreases in temperature (usually starting at 32° C.) until a cooling sensation or pain threshold is reached. The sensation of pleasant or innocuous cooling is typically elicited at temperatures of ˜23-29° C., while the sensation of cold pain is significantly variable, with multimodal distribution of the cold pain threshold recently reported, corresponding to modal threshold temperatures of 23.7° C., 13.2° C. and 1.5° C., respectively (Lotsch et al., PLoS One. 2015; 10(5):e0125822).

The cold plate test is one of the simplest assays to determine behavioral responses to both noxious and innocuous cold temperatures in both mice and rats. A number of endpoints can be obtained from the cold plate test, similar to the hot plate test. First, the response to a specific temperature (typically −5° C. to 15° C.) can be recorded (Allchorne et al., Mol Pain. 2005 Dec. 14; 10:36). Second, the number of flinches over a set period of time can be recorded at a specific temperature (Deuis et al., Pain. 2013 September; 154(9):1749-57). Third, aversive response to a cooling ramp can be used to determine the cold response threshold (Yalcin et al., J Pain. 2009 July; 10(7):767-73). The acetone evaporation test, first described in 1994, is a technique used to measure aversive behaviors triggered by evaporative cooling and is typically considered as a measure of cold allodynia (Carlton et al., Pain. 1994 February; 56(2):155-66; Choi et al., Pain. 1994 December; 59(3):369-76). The temperature preference is used as a surrogate measure of thermal aversion and aims to assess temperature preference in rodents. In its simplest form, the animal can choose between two adjacent areas maintained at different temperatures. This test is also referred to as the two-temperature choice assay or thermal place preference test and can be used to assess both cold or heat avoidance or preference (Moqrich et al., Science. 2005 Mar. 4; 307(5714):1468-72).

Non-Stimulus Evoked Nociception

In humans, spontaneous or background pain is pain that occurs without an identifiable stimulus. Spontaneous pain can be quantified in humans by asking them to describe their pain using a numeric pain scale (0-10), visual analog scale (transected line) or verbal scale (no pain to worst pain; Gaston-Johansson et al., J Pain Symptom Manage. 1990 April; 5(2):94-100; Wibbenmeyer et al., J Burn Care Res. 2011 January-February; 32(1):52-60). This cannot be done in rodents, making spontaneous pain difficult to quantify; however new methods to evaluate spontaneous pain are increasingly being reported, including grimace scales, burrowing assays, gait analysis, weight bearing and automated behavioral analysis (for a summary on behavioral tests used in non-stimulus evoked nociception, see Tappe-Theodor and Kuner, Eur J Neurosci. 2014 June; 39(11):1881-90). Grimace Scales

Facial expressions of mice can be used to score the subjective intensity of pain. In the Mouse Grimace Scale, five facial features are scored: orbital tightening, nose bulge, cheek bulge, ear position, and whisker position (Langford et al., Nat Methods. 2010 June; 7(6):447-9). Gait and weight bearing of rodents can be analyzed as a surrogate measure of nociception and are typically considered measures of non-evoked or stimulus-independent pain.

Therapeutic regimens comprising administration of one or more LEKTI protein domains (including one or more treatments with a microbe genetically modified to express one or more LEKTI protein domains encoded by one or more SPINK genes (alone or in combination with one or more additional treatment modalities)) can be tested in one or more animal models. While exemplary models are described herein, numerous additional animal models are well known in the art and can be similarly used. Treatment with one or more LEKTI protein domains can be compared, for example, to no treatment controls or control treatment with one or more current therapies. Additionally or alternatively, such models can be used to assess, for example, the effectiveness of a therapeutic regimen in which the frequency of treatments and/or the duration of each treatment are varied.

Additionally, therapeutic regimens comprising administration of one or more LEKTI protein domains (including one or more treatments with a microbe genetically modified to express one or more LEKTI protein domains encoded by one or more SPINK genes (alone or in combination with one or more additional treatment modalities)) can be tested in in vitro models (e.g., cell-based models, organ culture models). Further, such therapeutic regimens can be tested in vivo in human patients.

### 7. Diagnostic Methods

One aspect of the present invention is based on the idea that one or more LEKTI protein domains can be used to decrease the skin inflammatory response by decreasing expression and/or activation of pro-inflammatory cytokines via modulation of KLK5 activity and the downstream pathways that play a role in inflammation. Accordingly, in some embodiments, the disclosure provides methods for decreasing expression and/or activation of one or more pro-inflammatory cytokines. The method can be performed on cells or tissue explants cultured or otherwise maintained in vitro. In such in vitro embodiments, cells or tissue explants in culture are contacted with one or more LEKTI protein domains, as described throughout the application. The cells or tissue explants can be assessed to evaluate the decrease in expression and/or activation of one or more pro-inflammatory cytokines in comparison to untreated control. Exemplary pro-inflammatory cytokines that can be evaluated include, but are not limited to, TNFα, IL-1β, IL-6, IL-8, p38 MAPK, and other pro-inflammatory interleukins.

Suitable diagnostics methods can also be performed following in vivo treatment of tissues. Note that in this context the terms “in vitro” and “in vivo” are used to characterize the cells at the time of receiving treatment with the one or more LEKTI protein domains. Following treatment, the cells can be evaluated either in the context of the patient or animal or using an in vitro assay. The post-treatment evaluation method does not alter whether the delivery of the composition occurred in vivo or in vitro. In certain embodiments, one or more LEKTI protein domains is delivered to affected tissue of a patient in need thereof (delivered in vivo), and expression and/or activation of one or more pro-inflammatory cytokines is evaluated following treatment. Expression and/or activation of one or more pro-inflammatory-cytokines can be evaluated at any one or more time points following one or more treatments, and compared to expression and/or activation prior to initiation of treatment (but after the onset of symptoms of the inflammatory disorder). When used in this way, decrease in the local inflammatory response, as assessed by expression and/or activation of one or more pro-inflammatory cytokines, can be used to evaluate the progress of the treatment.

As indicated above, although the one or more LEKTI protein domains is delivered in vivo, analysis of the one or more pro-inflammatory cytokines can be conducted in vivo or in vitro. For example, for in vitro analysis, suitable tissue samples can be taken over time and analyzed in vitro. In the case of an inflammatory skin disorder, small skin samples can be taken for analysis. For in vivo analysis, vital dyes and agents can be used to help assess the inflammatory response in the tissue in its in vivo context and without the need to obtain a sample or biopsy from the patient. Regardless of whether the diagnostic step is conducted in vitro or in vivo, exemplary pro-inflammatory cytokines that can be evaluated include, but are not limited to, TNFα, IL-1β, IL-8, p38, other pro-inflammatory interleukins, and the like.

According to some embodiments, the diagnostic step is conducted multiple times throughout the course of treatment. In certain embodiments, the one or more diagnostic steps are used by a health care provider to help determine the duration of treatment, as well as whether the microbe genetically modified to express one or more LEKTI protein domains encoded by one or more SPINK genes should be used alone or combined with other therapies.

### 8. Kits

According to another aspect, the present disclosure provides a kit for the treatment or prevention of the effects of a skin disease or disorder of a mammal in need thereof comprising: (1) a composition comprising a microbe that is genetically modified to express one or more LEKTI protein domains encoded by one or more SPINK genes, wherein the LEKTI protein domains are effective to penetrate one or more layers of the mammal's skin and effective to inhibit serine protease activity of at least one serine protease in or on the mammal's skin; and (2) reagents for applying the composition to the skin of the mammal According to some embodiments, the microbes are adapted to live for a controlled duration on the surface of the mammal's skin and to provide a continuous supply of LEKTI protein domains.

According to another aspect, the present disclosure provides a kit for the treatment or prevention of the effects of a skin disease or disorder of a mammal in need thereof comprising: (1) a composition comprising a one or more LEKTI protein domains; and (2) reagents for applying the composition to the skin of the mammal According to some embodiments, the microbes are adapted to live for a controlled duration on the surface of the mammal's skin and to provide a continuous supply of LEKTI protein domains.

In addition to the above components, the subject kits will further include instructions for use of the components and/or practicing the subject methods. These instructions may be present in the subject kits in a variety of forms, one or more of which may be present in the kit. One form in which these instructions may be present is as printed information on a suitable medium or substrate, such as a piece or pieces of paper on which the information is printed, in the packaging of the kit, or in a package insert. Yet another means would be a computer readable medium, such as diskette, or CD, on which the information has been recorded. Further, another means by which the instructions may be present is a website address used via the internet to access the information at a removed site. Any convenient means may be present in the kits.

The components of the kits may be packaged either in aqueous media or in lyophilized form. The kits will generally be packaged to include at least one vial, test tube, flask, bottle, syringe or other container means, into which the described reagents may be placed, and preferably, suitably aliquoted. Where additional components are provided, the kit will also generally contain a second, third or other additional container into which such component may be placed.

The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

### EXAMPLES

The following examples are provided to further illustrate the methods of the present invention. These examples are illustrative only and are not intended to limit the scope of the invention in any way.

### Example 1

### Bacteria

According to some embodiments, bacteria of the Staphylococcus aureus RN4220 strain may be used in preparation of the vector (Kreiswirth, B N et al. 1983). According to some such embodiments, a stock solution of the strain is stored at −20° C. in 50% glycerol in LB or TS broth.

According to some embodiments, bacteria of the Staphylococcus epidermidis strain ATCC 12228 or NRRL B-4268 may be used (Zhang, YQ., et ah 2003). According to some such embodiments, a stock solution of the strain is stored at −20° C. in 50% glycerol in LB broth or TS broth. Bacteria are cultured in LB broth or TS broth. After 16 hours of incubation, bacteria are harvested by centrifugation and 10-fold concentrated in LB broth or TS broth at 2×10⁹ bacteria/100 ul. A stock preparation of the bacteria is prepared by inoculating 5 mL broth with S. epidermidis and grown overnight at 30° C. Then, 3 mL fully grown culture is added to 1 ml 60% glycerol and stored at −80° C.

Expression Vector

According to some embodiments, plasmid construct pKK30-LEKTI-complete may comprise the pKK30 vector with a LEKTI domain insert. According to some embodiments, the LEKTI domain may be operably linked to a SecA secretion signal, a 6×His tag (SEQ ID NO: 120), and/or an RMR cell permeation sequence, with expression under the control of a chloramphenicol-resistance (CmR) promoter sequence (from pDB114E). According to some embodiments, the pKK30 vector comprises a dihydrofolate reductase (dfrA) selection gene.

Transformation

According to some embodiments, a vector harboring the LEKTI sequence may be transformed into the S. epidermidis strain. The vector harboring the LEKTI sequence may be prepared/transformed comprising the steps of: preparation of competent S. aureus bacterial cells, transformation of S. aureus, isolation of plasmid DNA from S. aureus, preparation of competent S. epidermidis bacterial cells, transformation of S. epidermidis, growth of transformed S. epidermidis bacteria, and storage of transformed S. epidermidis.

According to some embodiments, alternative intermediate strains can also be used for transformation and isolation of plasmid DNA in preparation for transformation into S. epidermidis. These strains may include but are not limited to E. coli strains among other bacteria, including those deficient in methylation.

According to some embodiments, S. aureus RN4220 cells may be made electrocompetent by growing 50 ml culture overnight in LB or TS medium at 37° C., then inoculating 100 ml fresh LB or TS medium with 10 ml of overnight culture. When OD₆₀₀ reaches 0.2-0.3, cells are pelleted and resuspended with 1× volume of 4° C. 10% sucrose. This process is repeated 3×, and then the cells are resuspended with 0.1× volume of 4° C. 10% sucrose, pelleted, and resuspended with 1 ml of 10% sucrose.

For transformation of RN4220, 200-500 ug of LEKTI plasmid (e.g. pKK30-LEKTI-complete) may be mixed with electrocompetent cells and transformed using electroporation at room temperature at 2.5 kV using the MicroPulser Electroporator (Bio-Rad, Hercules, Calif.). Transformed cells are plated at 28° C. overnight on selective LB or TB medium, grown overnight in selective LB or TB medium and then used to isolate DNA.

According to some embodiments, electrocompetent S. epidermidis ATCC 12228 or NRRL B-4268 are made using the following methods. First, 50 ml overnight culture of ATCC 12228 or NRRL B-4268 from a −80° C. stock are grown at 37° C. in B2 medium (1.0% tryptone, 2.5% yeast extract, 0.5% glucose, 2.5% NaCl, 0.1% K₂PO₄, pH to 7.5). 10 ml of overnight culture is diluted into fresh pre-warmed B2 media and shaken until OD₆₀₀ reaches 0.5-0.6 and then pelleted for 10 min at 4° C. Next, cells are washed with 1, 1/2, 1/20, and 1/50 volumes of cold 10% glycerol, pelleting at 4° C. between washes. The final pellet is resuspended in 700 ul of cold 10% glycerol.

According to some embodiments, electrocompetent ATCC 12228 or NRRL B-4268 are transformed with pKK30-LEKTI-complete, isolated from S. aureus, using electroporation at 2.5 kV, 25 uF, 100Ω. (normal reading is 4.5-5 msec using the Micropulser Electroporator (Bio-Rad, Hercules, Calif.)). Cells are then plated at 28° C. on selective LB or TB medium. According to some embodiments, transformation of the bacteria can also be performed via alternative methods of transformation including but not limited to alternative intermediate strains, bacteriophage transduction, and heat shock.

Analysis of Protein Expression

According to some embodiments, transformed cells are fractionated and analyzed via SDS-PAGE electrophoresis and western blotting. Bacterial cells expressing recombinant LEKTI and bacterial control cells are pelleted and lysed with CelLytic B Cell Lysis Reagent (Sigma-Aldrich, St. Louis, Mo.). The supernatant from the induced sample is collected and concentrated. Samples are resuspended in a reduced sample buffer and then electrophoresed on a 4-15% Tris-acrylimide gel with Tris-HCL running buffer. Following electrophoresis, the gel is transferred to a PVDF membrane, and sequentially probed with a primary goat monoclonal antibody against LEKTI domains 8-11 or a His tag. A horseradish peroxidase-conjugated donkey anti-goat antibody (sc-2020) is then probed and the secondary antibodies detected through autoradiography (Syngene GeneGnome Bio Imaging System) using enhanced chemiluminescence substrate (SuperSignal West Pico, Thermo Scientific).

Analysis of the supernatant and cell lysate demonstrates the successful expression and secretion of the therapeutic polypeptide upon transformation with a plasmid containing the protein of interest. Detection of protein expression and secretion is also possible using alternative methods and the current example should not be construed as a limitation to the present invention.

Treatment of Human Subjects

According to some embodiments, 1×10⁹ colony forming units (CFU) of S. epidermidis containing recombinant LEKTI can be added to a pharmaceutically acceptable carrier. The foregoing composition is useful for treating or preventing inflammatory skin diseases or disorders in a subject in need thereof. The composition can be applied at least once per day, up to for example about 3 to 4 times per day, or as needed or prescribed. According to some embodiments, only a single application is required to achieve a therapeutic effect. The composition can be used for as long as needed to ensure treatment of the condition or to continue to prevent the condition. The duration of treatment can vary from about 1 day up to about 10 to 14 days or longer. In certain instances, long term or chronic treatment can be administered.

### Example 2

### Testing Serine Protease Inhibition Activity of Recombinant LEKTI

According to some embodiments, the protease inhibition activity of recombinant LEKTI is tested for differences achieved when operably linked to various secretion peptides and cell penetration peptides. According to some embodiments, specific combinations of secretion peptides and cell penetration peptides may have unpredictable effects on the protease inhibition function of the LEKTI domains, and therefore may be determined empirically.

According to some embodiments, LEKTI domains D8-D11, operably linked to a secretory tag, 6×His tag (SEQ ID NO: 120), and/or cell penetration tag, are cloned into an insect expression vector for large scale production of purified recombinant protein and assessed for inhibitory activity on one or more proteases (e.g. plasmin, cathepsin G, elastase, and trypsin).

Insect Cells and Reagents

The following reagents may be obtained commercially as indicated: Fall Army worm cell line Spodoptera frugiperda (Sf9), low-melting point agarose, cellFECTIN, pFASTBAC1, pCRII-TOPO, Escherichia coli competent DH10BAC, cabbage looper egg cell line Trichoplusia ni 5B1-4 (High Five), and ultimate serum-free insect medium from Invitrogen (Carlsbad, Calif.); restriction endonucleases from New England Biolabs (Beverly, Mass.); TALON Superflow from Clontech Laboratory (Palo Alto, Calif.); Insect-XPRESS medium and fetal bovine serum from BioWhittaker (Walkersville, Md.); YM10 Centriplus from Millipore Corp. (Bedford, Mass.); precast SDS-PAGE gels, protein assay kit, SEC-250 size column, and prestained markers from Bio-Rad (Hercules, Calif.); BSA from Kabi Pharmacia (Franklin, Ohio); DTT and glycerol from Boehringer Mannheim Biochemicals (Indianapolis, Ind.); and penta-His mAb and six-His (SEQ ID NO: 120) tagged protein ladder from QIAGEN Inc. (Valencia, Calif.).

Cloning and Expression of LEKTI D8-D11

6×His (SEQ ID NO: 120) tagged LEKTI domains (e.g. D6, SEQ ID NO: 109) operably linked to various permutations of secretion peptides and cell penetration peptides may be cloned into the pFASTBAC1 vector according to the manufacturers' instructions. Recombinant LEKTI composite viruses are then generated as previously described by Gao, M. et al., (1996) J. Biol. Chem. 271, 27782-27787, which is incorporated herein by reference in its entirety. To test the recombinant LEKTI composite viruses for recombinant LEKTI expression, Sf9 cells may be infected at varying multiplicities of infection with recombinant viruses, and the cell lysate and medium collected every 24-96 h. The presence of histidine-tagged protein may be confirmed by Western blot analysis using penta-His mAb directed against the six-histidine tag (SEQ ID NO: 120) as per the manufacturer's recommendations. LEKTI composite viruses that displayed the highest level of expression may be chosen for further experiments and spinner flasks.

The recombinant LEKTI protein may be produced on a large scale by infecting spinner cultures of Sf9 cells (1.6 billion cells) in 10% serum containing Insect-XPRESS medium at a multiplicity of infection of 8 plaque forming units (PFU). Three days after infection, the cell pellet may be harvested and the recombinant LEKTI selectively purified from the cell lysate using a Co²⁺-charged Sepharose affinity column (TALON) followed by SEC-250 size column chromatography, as previously described in Jayakumar, A. et al., (1995) Proc. Natl. Acad. Sci. U.S.A. 92, 8695-8699. Fractions containing homogeneous LEKTI may be pooled and concentrated by ultrafiltration. Protein may be quantified using the Bio-Rad Protein Assay Kit II.

Protease Inhibition Assay Reagents and Protocol

The following enzymes, chromogenic substrates, and reagents may be obtained commercially as indicated: human plasmin, human cathepsin L, human cathepsin S, human trypsin, human cathepsin G, human chymotrypsin, and human neutrophil elastase (HNE) from Athens Research & Technology, Inc. (Athens, Ga.); subtilisin A from Calbiochem-Novabiochem (San Diego, Calif.); papain from Roche Molecular Biochemicals (Indianapolis, Ind.); furin from New England BioLabs; succinyl-Ala-Ala-Pro-Phe-p-nitroanilide (Succ-AAPF-pNA), succinyl-Ala-Ala-Val-pNA (Succ-AAVpNA), and D-Val-Leu-Lys-pNA (VLK-pNA) from Sigma Chemical Co. (St. Louis, Mo.); H-Glu-Gly-Arg-pNA (EGRpNA) and benzyloxycarbonyl-Phe-Arg-pNA (Z-FR-pNA) from Bachem Bioscience, Inc. (King of Prussia, Pa.); and methoxy-Succ-Arg-Pro-Tyr-pNA (MeO-Succ-RPY-pNA) from Chromogenix Instrumentation Laboratory SpA (Milan, Italy). PBS reaction buffer (137 mM NaCl, 27 mM KCl, and 10 mM phosphate buffer (pH 7.4)) may be used with trypsin, plasmin, cathepsin G, HNE, and chymotrypsin. Cathepsin reaction buffer (0.1% CHAPS, 50 mM sodium acetate (pH 5.5), 1 mM EDTA) may be used with cathepsins K, L, and S and papain. A unique reaction buffer may be used with subtilisin A (PBS and 0.1% Tween 20).

Proteinase inhibitory activity may be detected by the ability of recombinant LEKTI to block the cleavage of small, chromogenic peptide substrates as determined by a spectroscopy technique described previously in Schick, C. et al., (1998) Biochemistry 37, 5258-5266, which is incorporated herein by reference in its entirety. Inhibition of proteinase may be assessed after preincubating the enzyme with recombinant LEKTI for 2 min at 25° C. in 100 uL of assay buffer. This mixture may be added to 890 or 880 uL of assay buffer in a 1 mL quartz cuvette. The proteinase activity may be initiated by adding 10-20 uL of the appropriate pNA substrate. The change in absorbance at 405 nm (A₄₀₅=8.8 10⁻³ M⁻¹ cm⁻¹) may be followed for as long as 10 min using a spectrophotometer (Beckman Instruments, Inc., Fullerton, Calif.). The rate changes (ΔA₄₀₅/min) of inhibited and control reactions may be determined from velocity plots.

According to some embodiments, different combinations of secretory tag and cell penetration tag may cause differing LEKTI protease activity on each of the tested proteases (e.g. trypsin, plasmin, cathepsin G, HNE, subtilisin A, and chymotrypsin). Furthermore, discrete combinations of secretory tag and cell penetration tag may cause differing LEKTI protease activity among individual proteases.

### Example 3. Penetrating Peptide Mediated Delivery

According to some embodiments, various combinations of secretory tag and cell penetration tag may affect the ability of the recombinant LEKTI protein to pass through a cell membrane to a greater or lesser degree. Thus, the various recombinant LEKTI products may be tested in cell culture to assess the effect of the various combinations of secretory tag and cell penetration tag.

According to some embodiments, adherent fibroblastic HS-68, NIH-3T3, 293, Jurkat T, or Cos-7 cell lines may be cultured in Dulbecco's modified Eagle's medium (DMEM) supplemented with 1% (vol/vol) 200 mM glutamine, 1% (vol/vol) antibiotics (streptomycin, 10,000 μg/ml; penicillin, 10,000 IU/ml), and 10% (wt/vol) FBS, at 37° C. in a humidified atmosphere containing 5% CO2. For peptide-mediated delivery of recombinant LEKTI proteins, purified recombinant LEKTI product (as obtained above) may be loaded in DMEM or PBS (500 μl of DMEM containing 0.25 μg of protein) and incubated for 30 min at 37° C. Cells grown to 75% confluency are then overlaid with these recombinant LEKTI protein media. After 30 min incubation at 37° C., 1 ml of fresh DMEM supplemented with 10% FBS is added to the cells, without removing the overlay of recombinant LEKTI protein, and cells are returned to the incubator for another 30 min. Cells are then extensively washed with PBS and examined for recombinant LEKTI protein. Cells could be observed by immunofluorescence by first fixing with 2% formalin (Sigma), permeabilizing, then incubating with primary anti-6×His tag (SEQ ID NO: 120) antibody and secondary antibody according to the manufacturers' instruction (“6×His” disclosed as SEQ ID NO: 120). Alternatively cells lysates could be obtained and the presence of His tagged recombinant LEKTI observed via Western blot, as described above.

According to some embodiments, certain combinations of secretory protein and penetrating peptide have differing effects on the ability of the recombinant LEKTI protein's ability to pass through the cell membrane.

### Example 4. Effects of LEKTI D6 Domain on KLK5 Provoked 3D Skin Model

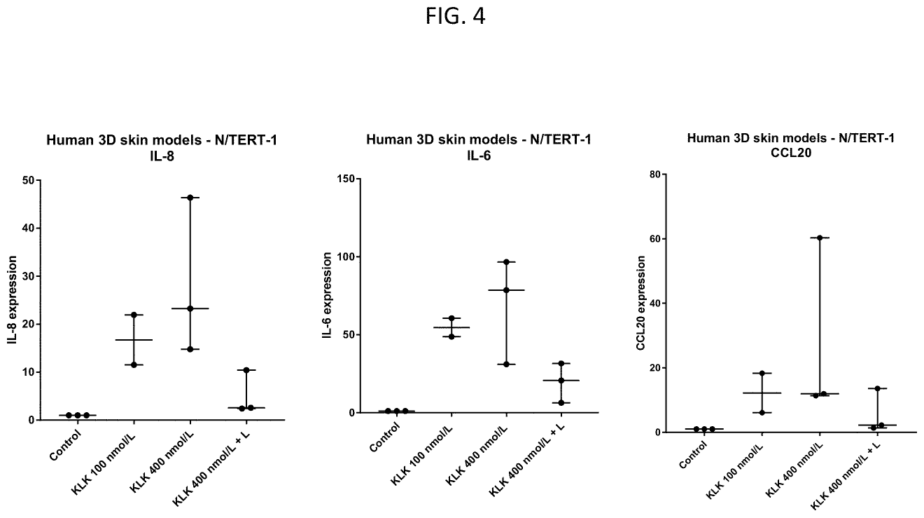

The effects of KLK5 on the expression level of pro-inflammatory cytokines IL-6, IL-8 and chemokine CCL-20 on 3D human skin equivalents (Smits et al., Immortalized N/TERT keratinocytes as an alternative cell source in 3D human epidermal models. Sci Rep. 2017 Sep. 19; 7(1):11838), and the inhibitory activity of LEKTI D6 was examined Immortalized keratinocyte cell line N-TERT, primary fibroblasts and endothelial cells were used. Cultures were grown for 4 weeks and then stimulated with KLK-5 (100 nmol/l) and PAR-2 (400 nmol/l), plus one construct with 10 min LEKTI D6 preincubation (1 μmol/l). LEKTI D6 was purified from SE. LEKTI D6 treatment was repeated after 30 min and 60 min. RNA was isolated 6 hours after trypsin stimulation. Levels of inflammatory cytokines IL-6, IL-8 and CCL-20 were quantified real time qPCR. Results are shown in FIG. 4. GAPDH housekeeping enzyme was used to normalize gene expression data. As shown in FIG. 4, human KLK 5 stimulates human 3D construct, and LEKTI D6 (+L) can inhibit this stimulation.

### Complex 3 Dimensional Skin Construct

Two human keratinocyte cell lines (N/TERT1 and N/TERT2G) suitable for 3-D-skin constructs were obtained (Smits et al., 2017). Fibroblasts, endothelial cells and keratinocytes were co-cultured in an air liquid interface for 2 weeks, and embedded for electron-microscopic analysis. The three-dimensional culturing of human keratinocytes at the air-liquid interface yields a fully stratified epidermis including a functional stratum corneum. The effects of LEKTI D6 on epidermal structure and function in pathological states (e.g., inflammation), will be examined.

### Example 5. Effects on LEKTI D6 Domain on Thymic Stromal Lymphopoietin (TSLP) Expression

TSLP acts as a potent stimulator of Th2 cytokines including interleukins (IL) 4, 5, and 13, that, in turn, trigger IgE production and release from plasma cells (Brandt and Sivaprasad, 2011). It has been reported that signaling between epithelial cells and innate immune cells via TSLP is thought to drive atopic dermatitis (AD), and that epithelial cells directly communicate with cutaneous sensory neurons via TSLP to promote itch (Bautista et al., Cell 155(2) October 2013).

Here, the effect of LEKTI D6 on TSLP expression was examined Murine keratinocyte cell line (MSC-P5) was grown to confluency, and cultivated on the day of treatment without fetal calf serum (FCS).

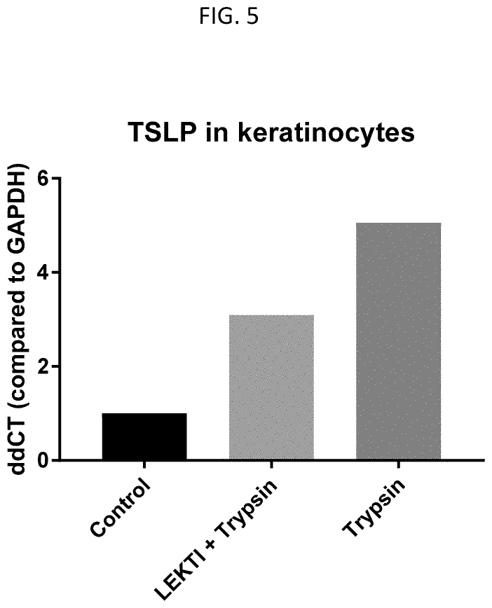

Cells were pre-incubated with LEKTI domain 6 (D6) (800 nmol/l+400 nmol 30 min later, +400 nmol 60 min later). Cells were stimulated with trypsin (Meyer Hoffert et al., 2004), PAR-2 activation (5-50 nmol/l), 10 min after first LEKTI administration. RNA was isolated 6 hours after trypsin stimulation. Results are shown in FIG. 5. GAPDH housekeeping enzyme was used to normalize gene expression data. As shown in FIG. 5, trypsin stimulated TSLP expression, and LEKTI D6 inhibited this stimulation.

### Example 6. Effect of LEKTI D6 on Inflammatory Response

Murine bone marrow cells were isolated and grown in the presence of recombinant mouse GM-CSF to yield conventional dendritic cells (DCs). On day 9 (roughly 90% purity) cells were pre-incubated with LEKTI domain 6 (D6) (400 nmol/1+200 nmol 30 minutes later). Cells were stimulated with trypsin and PAR-2 activation (100 nmol/l) at 10 min after first LEKTI administration. RNA was isolated 6 hours after trypsin stimulation.

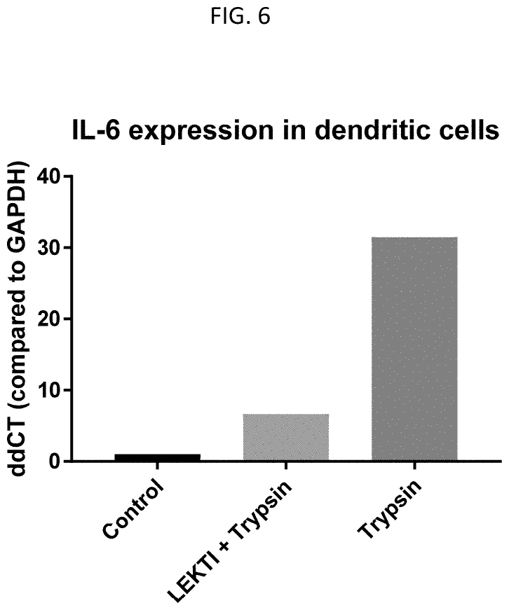

IL-6 response by dendritic cells is a typical pro-inflammatory response to PAR-2 activation (Dekita et al., Front Pharmacol. 2017 Jul. 17; 8:470). Thus, real-time qPCR was carried out to determine IL-6 expression levels in the presence and absence of LEKTI D6. Results are shown in FIG. 6. GAPDH housekeeping enzyme was used to normalize gene expression data. As shown in FIG. 6, LEKTI D6 inhibits IL-6 expression in dendritic cells as determined by realtime qPCR. Thus, these results demonstrated that the presence of LEKTI D6 inhibited the inflammatory response, further supporting a role for LEKTI D6 as a therapeutic in controlling the inflammatory response.

### Example 7. In Vivo Model to Test Effect of LEKTI D6 on Inflammatory Disease or Disorder

Efficacy of therapeutic LEKTI D6 S. epidermidis strains will be evaluated in a mouse model of an inflammatory skin disease or disorder. Mice will be treated with topical application of recombinant LEKTI to resolve skin conditions in the chosen model. The rationale for first using purified LEKTI is to avoid dependency on the construction of S. epidermidis strains such that the efficacy of topical application in vivo can be rapidly demonstrated. Second, we will evaluate the ability of S. epidermidis-purified or LEKTI to demonstrate the value of probiotic colonization for sustained remediation. To assess the effect of LEKTI D6 in the mouse model, we will perform longitudinal assays (1×/week) where possible and endpoint assays (3 weeks post-colonization) to test if application of therapeutic S. epidermidis will (1) produce detectable amounts of LEKTI in vivo, as measured by immunohistochemical analysis of skin (endpoint), (2) reduce symptoms and/or severity as measured by DASI (longitudinal and endpoint), (3) improve TEWL (longitudinal) and permeability scores (endpoint), (4) ameliorate skin morphology, as measured by histological analysis (endpoint), and (5) result in changes in proteolytic activity, as measured using colorimetric assays that target KLK5.

### Example 8. In Vivo Model of Pruritus

Efficacy of therapeutic LEKTI D6 S. epidermidis strains will be evaluated in a mouse model of pruritus. Mice will be treated with topical application of recombinant LEKTI to resolve skin conditions in the chosen model. The rationale for first using purified LEKTI is to avoid dependency on the construction of S. epidermidis strains such that the efficacy of topical application in vivo can be rapidly demonstrated. Second, we will evaluate the ability of S. epidermidis-purified or LEKTI to demonstrate the value of probiotic colonization for sustained remediation. To assess the effect of LEKTId6 in the mouse model, we will perform longitudinal assays (1×/week) where possible and endpoint assays (3 weeks post-colonization) to test if application of therapeutic S. epidermidis will (1) produce detectable amounts of LEKTI in vivo, as measured by immunohistochemical analysis of skin (endpoint), (2) reduce symptoms and/or severity of itch, (3) improve TEWL (longitudinal) and permeability scores (endpoint), (4) ameliorate skin morphology, as measured by histological analysis (endpoint), and (5) result in changes in proteolytic activity, as measured using colorimetric assays that target KLK5.

### Example 9. Effect of LEKTI D6 on Activation of Ion Channels Associated with Itch

Transient receptor potential (TRP) channels comprise 28 members in mammals that can be divided into six subfamilies based on amino acid sequence homology, including TRPA, TRPC, TRPM, TRPML, TRPP, and TRPV (Montell et al. Cell. 2002 Mar. 8; 108(5):595-8). TRP channels are molecular sensors of mechanical, chemical, and thermal environmental cues and are crucially involved in both acute and chronic itch (Dong. Neuron. 2018 May 2; 98(3):482-494). Six TRP channels are now firmly associated with itch generation and transduction. They are implicated in many sensory functions including taste, smell, thermoception, touch, osmolarity, and pain (Venkatachalam K, Montell C Annu Rev Biochem. 2007; 760:387-417; Zheng J Compr Physiol. 2013 January; 3(1):221-4; Wu et al. Pharmacol Rev. 2010 September; 62(3):381-404). In the past two decades, numerous studies have demonstrated that TRP channels are critically involved in itch sensation under both physiological and pathological conditions (Steinhoff M, Biro T J Invest Dermatol. 2009 March; 129(3):531-5; Moore et al. Neurosci Bull. 2018 February; 34(1):120-142).

TRPV1 belongs to a subfamily of temperature-sensitive TRP channels, also called “ThermoTRPs” (Kim et al. PLoS One. 2013; 8(3):e59593). TRPV1 is activated by noxious temperatures (>43° C.) (Rosenbaum T., Simon S. A. In: Liedtke W. B., Heller S., editors. CRC Press; Boca Raton, Fla., USA: 200). In addition, TRPV1 is activated by capsaicin, low pH, and numerous molecules associated with inflammation and tissue damage such as bradykinin, prokineticin, prostaglandins, anandamide, and retinoids (Luo et al. Cell Mol Life Sci. 2015 September; 72(17):3201-23; Carnevale V, Rohacs T. Pharmaceuticals (Basel). 2016 Aug. 23; 9(3); Yin et al. J Clin Invest. 2013 September; 123(9):3941-51). As the best-characterized itch mediator, histamine is released from mast cells and binds H1/H4 receptors on skin nerve terminals to elicit itch (Shim, W. S., and Oh, U. 2008. Mol. Pain 4:29), via activation the PLCbeta3 and transient receptor potential subtype VI (TRPV1)(Imamachi et al. Proc. Natl. Acad. Sci. U. S. A 106: 11330-11335).

In this example, the effect of LEKTI D6 on inhibition of the activation of the ion channels TRPV1 and TRPV4 with KLK4 was examined.

TRPV1 and TRPV4 cells were seeded at 20,000 cells per well for 24 hours±6 hours on poly-d-lysine plates. All reference compounds were made up at 300× final concentration in PBS or DMSO. Purified recombinant LEKTI D6 (Rec LEKTI-d6 S1-DM21/70) was prepared in PBS at 4×, then serially diluted in HBSS+HEPES.

For TRPV1, the agonist used was capsaicin (tested at 3 μM top concentration with half log dilution) and the antagonist used was capsazepine (tested at 10 μM top concentration with half log dilution). For TRPV4, the agonist used was GSK1016790A (tested at 0.1 μM top concentration with half log dilution) and the antagonist was Ruthenium Red (tested at 1 μM top concentration with half log dilution). Purified recombinant LEKTI D6 was tested at 10 μM top concentration with half log dilution.

Cells were dye loaded using FLIPR Dye Buffer and incubated at 30° C. for 90 minutes. Compounds were incubated following dye loading for an additional 5 minutes at room temperature or 30° C., for TRPV1 and TRPV4, respectively.

The plate was read on the FLIPR Tetra, then the second addition of “EC₈₀)” KLK14 (30 nM) was added and read for an additional three minutes. This step was completed at room temperature within the machine. Maximum fluorescence/luminescence values were exported and data manipulated to calculate percentage activation and percentage inhibition. Data manipulation calculation is as followed:

((Max RLU)−(Baseline Avg.))/((Positive Avg.)−(Baseline Avg.))

Results for TRPV1 agonist and antagonist treatment are shown in FIG. 7A and FIG. 7B. Antagonist treatment was combined with LEKTI D6 treatment. When cells were pre-incubated with purified recombinant LEKTI D6 (Rec LEKTI-d6 S1-DM21/70), activity in any of the compounds or buffers presented in the pre-incubation assay was not observed, as shown in the table below.

| | Concentration | % inhibition | | |
|---|---|---|---|---|
| Client Compound ID | (μM) | mean | SEM | n |
| Rec LEKTI-d6 S1-DM21/70 | 0.003 | −4.0 | 5.7 | 5 |
| Rec LEKTI-d6 S1-DM21/70 | 0.01 | 21.6 | 6.3 | 5 |
| Rec LEKTI-d6 S1-DM21/70 | 0.03 | −11.1 | 13.3 | 5 |
| Rec LEKTI-d6 S1-DM21/70 | 0.1 | 12.4 | 7.4 | 5 |
| Rec LEKTI-d6 S1-DM21/70 | 0.3 | 16.8 | 16.9 | 5 |
| Rec LEKTI-d6 S1-DM21/70 | 1 | 46.9 | 19.3 | 5 |
| Rec LEKTI-d6 S1-DM21/70 | 3 | 56.4 | 16.0 | 5 |
| Rec LEKTI-d6 S1-DM21/70 | 10 | 39.3 | 1.9 | 5 |

| | | % inhibition | | |
|---|---|---|---|---|
| Client Compound ID | v/v (%) | mean | SEM | n |
| Activation Buffer | 1.67% | 75.9 | 6.7 | 5 |
| Activation Buffer + Themolysin | 1.67% | 91.8 | 1.9 | 5 |

| | conc | Mean | | | | % |
|---|---|---|---|---|---|---|
| ID | (uM) | RLU | SD | SEM | n | CV |
| KLK14 | 0.03 | 852.26 | 157.06 | 49.67 | 10 | 18.43 |
| Min DMSO Vehicle | 0.003 | 47.00 | 15.33 | 2.37 | 42 | 32.61 |
| Max − Min | | 805.26 | | | | |

These results suggest that any activity seen in the antagonist assay was not due to desensitization of the TRPV1 channel.

Results for TRPV4 agonist and antagonist treatment are shown in FIG. 8A and FIG. 8B. Antagonist treatment was combined with LEKTI D6 treatment. When cells were pre-incubated with purified recombinant LEKTI D6 (Rec LEKTI-d6 S1-DM21/70), activity in any of the compounds or buffers presented in the pre-incubation assay was not observed, as shown in the tables below.

| | Concentration | % inhibition | | |
|---|---|---|---|---|
| Client Compound ID | (μM) | mean | SEM | n |
| Rec LEKTI-d6 S1-DM21/70 | 0.003 | −24.1 | 2.5 | 5 |
| Rec LEKTI-d6 S1-DM21/70 | 0.01 | −11.0 | 9.8 | 5 |
| Rec LEKTI-d6 S1-DM21/70 | 0.03 | 9.9 | 11.6 | 5 |
| Rec LEKTI-d6 S1-DM21/70 | 0.1 | 6.4 | 7.0 | 5 |
| Rec LEKTI-d6 S1-DM21/70 | 0.3 | 1.5 | 10.3 | 5 |
| Rec LEKTI-d6 S1-DM21/70 | 1 | 6.3 | 4.0 | 5 |
| Rec LEKTI-d6 S1-DM21/70 | 3 | 26.7 | 17.4 | 5 |
| Rec LEKTI-d6 S1-DM21/70 | 10 | 73.0 | 10.8 | 5 |

| | | % inhibition | | |
|---|---|---|---|---|
| Client Compound ID | v/v (%) | mean | SEM | n |
| Activation Buffer | 1.67% | 20.4 | 7.7 | 5 |
| Activation Buffer+ | 1.67% | 8.6 | 4.1 | 5 |

| | conc | Mean | | | | % |
|---|---|---|---|---|---|---|
| ID | (uM) | RLU | SD | SEM | n | CV |
| KLK14 | 0.03 | 1997.21 | 439.07 | 98.18 | 20 | 21.98 |
| Min DMSO Vehicle | 0.003 | 83.44 | 173.74 | 26.81 | 42 | 208.22 |
| Max − Min | | 1913.76 | | | | |

These results suggest that any activity seen in the antagonist assay was not due to desensitization of the TRPV4 channel.

Taken together, these results demonstrate that LEKTI D6 was effective in inhibiting the activation of ion channels associated with itch.

### Example 10. In Vivo Model of Neuropathic Pain

Efficacy of therapeutic LEKTI D6 S. epidermidis strains will be evaluated in a mouse model of neuropathic pain. Mice will be treated with recombinant LEKTI to resolve pain in the chosen model.

The Seltzer model of neuropathic pain will be used with therapeutic LEKTI D6 S. epidermidis strains in C57BL/6 male mice (Seltzer et al. Pain. 1990 November; 43(2):205-18). This neuropathic pain model produces a partial nerve injury by tying a tight ligature with a 7-0 silk suture around approximately ⅓ to ½ the diameter of the sciatic nerve of one single thigh per mouse. Post-surgery, the mice will be allowed to recover for at least two days and then will be studied for several weeks post-surgery for thermal hyperalgesia in the Hargreaves' test and for mechanical allodynia in the von Frey test (Deuis et al. Front Mol Neurosci. 2017; 10: 284). Following the Seltzer surgery, mice will be tested at day 4 and day 7 post-surgery to confirm that the pain developed. Then, at day 7 post-surgery following the behavioral pain testing the mice will be treated with the therapeutic LEKTID6 S. epidermidis strains or a suitable control.

The ability of S. epidermidis-purified or LEKTI to demonstrate the value of probiotic colonization for sustained remediation will also be evaluated. To assess the effect of LEKTID6 in the mouse model, we will test if application of therapeutic S. epidermidis will produce detectable amounts of LEKTI in vivo, as measured by immunohistochemical analysis of skin (endpoint) and result in changes in proteolytic activity, as measured using colorimetric assays that target KLK5.

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

Although illustrative embodiments of the present invention have been described herein, it should be understood that the invention is not limited to those described, and that various other changes or modifications may be made by one skilled in the art without departing from the scope or spirit of the invention.

1. A method of treating or preventing a skin disease or disorder in a subject in need thereof, comprising administering one or more LEKTI protein domains to the skin of the subject in need thereof, wherein the one or more LEKTI protein domains provides a therapeutic effect to decrease one or more symptoms of the skin disease or disorder.

2. (canceled)

3. The method of claim 1, wherein administering the one or more LEKTI protein domains comprises administering a microbe genetically modified to express one or more LEKTI protein domains encoded by one or more SPINK genes to the skin of the subject in need thereof, wherein the one or more LEKTI protein domains provides a therapeutic effect to decrease one or more symptoms of the skin disease or disorder.

4. (canceled)

5. The method of claim 3, wherein the one or more SPINK genes are selected from the group consisting of: SPINK1, SPINK2, SPINK4, SPINK5, SPINK6, SPINK7, SPINK8, SPINK9, SPINK13, and SPINK14.

6. The method of claim 3, wherein the one or more SPINK genes encodes a LEKTI protein, and protein domains thereof, selected from LEKTI, LEKTI-2 and LEKTI-3.

7. The method of claim 1, wherein the skin disease or disorder is an inflammatory skin disease or disorder.

8. The method of claim 1, wherein the skin disease or disorder is pruritus.

9. The method of claim 1, wherein the skin disease or disorder is pain or manifests with a symptom of pain.

10. The method of claim 1, wherein the one or more symptoms are selected from one or more from the group consisting of: inflammation, pain, itching, skin dryness, skin flaking, bacterial count, number of skin lesions, severity of skin lesions, frequency of outbreaks of skin lesions, redness, skin discoloration and expression of an inflammatory cytokine.

11.-16. (canceled)

17. The method of claim 9, wherein treating or preventing the pain comprises treating or preventing the symptoms of pain.

18. The method of claim 9, wherein the pain is selected from the group consisting of: acute or chronic pain, nociceptive pain, neuropathic pain, traumatic pain, inflammatory pain, post-operative incision pain, pain associated with cancer, neuropathic pain, fracture pain, osteroporotic fracture pain, bone cancer pain and gout joint pain.

19. (canceled)

20. (canceled)

21. (canceled)

22. A method of preventing or reducing scarring associated with an inflammatory skin disease or disorder in a subject in need thereof, comprising administering one or more LEKTI protein domains to the skin of the subject in need thereof, wherein the one or more LEKTI protein domains penetrates the skin to provide a therapeutic effect to prevent or reduce scarring associated with the inflammatory skin disease or disorder.

23. The method of claim 22, wherein administering the one or more LEKTI protein domains comprises administering a microbe genetically modified to express one or more LEKTI protein domains encoded by one or more SPINK genes to the skin of the subject in need thereof.

24. The method of claim 22, wherein the inflammatory skin disease or disorder is selected from the group consisting of: rosacea, psoriasis and atopic dermatitis.

25. A method for decreasing the number of skin lesions in a subject with an inflammatory skin disease or disorder, comprising administering one or more LEKTI protein domains to the skin of the subject in need thereof, wherein the one or more LEKTI protein domains penetrates the skin to provide a therapeutic effect to decrease the number of skin lesions on skin of the subject.

26. The method of claim 25, wherein administering the one or more LEKTI protein domains comprises administering a microbe genetically modified to express one or more LEKTI protein domains encoded by one or more SPINK genes to the skin of the subject in need thereof.

27. The method of claim 25, wherein the inflammatory skin disease or disorder is selected from the group consisting of: rosacea, psoriasis and atopic dermatitis.

28. A method for treating or preventing itch as a symptom or sensation associated with a disease or disorder in a subject, comprising administering one or more LEKTI protein domains to the subject to provide a therapeutic effect.

29. The method of claim 28, wherein administering the one or more LEKTI protein domains comprises administering a microbe genetically modified to express one or more LEKTI protein domains encoded by one or more SPINK genes to the skin of the subject.

30. A method of treating or preventing one or more symptom or sensation arising from an irritation, hives, pain, inflammation, asthma, allergy, or allergic rhinitis in a subject, comprising administering one or more LEKTI protein domains to the subject to provide a therapeutic effect.

31. The method of claim 30, wherein administering the one or more LEKTI protein domains comprises administering a microbe genetically modified to express one or more LEKTI protein domains encoded by one or more SPINK genes to the skin of the subject.

32.-38. (canceled)

39. The method of claim 1, wherein the one or more LEKTI protein domains are encoded by a nucleic acid.

40. The method of claim 39, wherein the nucleic acid comprises a sequence that is at least 85% identical to SEQ ID NO: 119 or SEQ ID NO: 128, or fragments thereof.

41. The method of claim 39, wherein the nucleic acid is comprised in a vector.

42. The method of claim 41, wherein the vector is a viral expression vector.

43. The method of claim 42, wherein the vector is comprised within a cell.

44.-50. (canceled)

51. The method of claim 1, wherein the at least one LEKTI domain comprises an amino acid sequence comprising any one of SEQ ID NOs 104-118.

52.-55. (canceled)

56. The method of claim 3, wherein the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp., and mixtures thereof.

57. A recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide.

58. A pharmaceutical composition comprising the recombinant microorganism of claim 57.
